(19) United States
(12) Patent Application Publication
(10) Pub. No.: US 2022/0002663 A1
(43) Pub. Date: Jan. 6, 2022

(54) SELECTIVE SALMONELLA OR E. COLI CULTIVATION METHOD, COMPOSITIONS AND USES

(71) Applicant: SOLUS SCIENTIFIC SOLUTIONS LTD, East Kilbride (GB)

(72) Inventors: David Simon Illingworth, Mansfield (GB); Nevin Perera, East Kilbride (GB); Paul Wells, East Kilbride (GB); David Higgins, East Kilbride (GB)

(21) Appl. No.: 17/277,383

(22) PCT Filed: Sep. 18, 2019
(86) PCT No.: PCT/GB2019/052626

§ 371 Date: Mar. 18, 2021

(30) Foreign Application Priority Data
Sep. 20, 2018 (GB) 1815358.5

(51) Int. Cl. C12N 1/20; G01N 33/569

(52) U.S. Cl. CPC C12N 1/20; G01N 33/56916

(57) ABSTRACT

The present disclosure provides rapid, convenient and sensitive means to detect low abundance target Gram negative bacteria such as Salmonella contamination from a range of samples, including food samples and environmental samples, through a single-step culture method and downstream sensitive detection.

### FIELD

The disclosure relates to the field of microbial detection.

### BACKGROUND

Because food products are biological in nature, they are capable of supporting the growth of a variety of contaminating microorganisms. In the United States, an estimated 76 million cases of foodborne illness occur each year costing between $6.5 and $34.9 billion dollars in medical care and lost productivity (Buzby and Roberts, 1997; Mead et al, 1999). In Europe it has been estimated that the economic and health care costs of Salmonella are between 620 million and €3 billion euros (David Byrne, European Commissioner for health and consumer protection, 2000).

Salmonella, Listeria, Campylobacter, Escherichia coli O157:H7 and Shigella are responsible for the majority of cases of foodborne illness. For example, Salmonella and Listeria alone were responsible for 31% and 28% respectively of food-related deaths (Mead et al, 1999) and in Japan, salmonellosis accounted for over 14% of the total foodborne illness outbreaks between 1981 and 1995 (Lee et al, 2001). In fact, it has been estimated that bacteria are the causative agents of as much as 60% of the cases of foodborne illness requiring hospitalisation. As a result, one of the biggest contributors to waste is delay caused by inefficient and slow testing of products for microbial contamination. With various testing methods, manufacturers must wait from three to seven days for the results of microbial incubation. The costs arising from such delays are significant—reducing supply chain efficiency, tying up inventory and increasing spoilage.

The costs of inadequate or insufficient testing can be as, if not more, costly. For example, in 1999, it cost Sara Lee an estimated $76 million in costs related to the recall of 35 million pounds of hot dogs and deli meats at its Bil Mar Foods unit, after the food was linked to an outbreak of Listeria. According to ‘The Scotsman’, contamination of chocolate with Salmonella in 2006 cost Cadbury Schweppes an estimated £20 million in recall costs, advertising, lost revenue and subsequent improvements to its manufacturing operation. More recently in 2009, the Peanut Corporation of America, a company with an estimated $25 million in sales in 2008, filed for bankruptcy after being identified as the source of a major Salmonella outbreak in peanuts in the USA.

Therefore, detection of the presence of pathogenic microorganisms such as Salmonella, E. coli, Shigella and Listeria in food, feed and environmental samples is of great economic importance. However, conventional culture methods for detection of such microorganisms are both labour intensive and time-consuming. Often such methods rely on standard processes that have been in use for more than 50 years.

In addition, pathogenic microorganisms can persist for long periods in an environment in a heavily stressed state known as ‘viable but not culturable (VNC)’ or ‘not immediately culturable (NIC)’. Such heavily stressed microorganisms show only a weak metabolic activity, often at the limits of detection, and they lose the ability to form colonies on non-selective plating media or to grow in non-selective broth media (Reissbrodt et al, 2002). However, when such non-culturable colonies exist in food and animal feed, they may still be capable of causing disease if ingested. This poses particular problems with regard to detection since such stressed microorganisms may not be revived sufficiently to be detected, resulting in false negative results.

Furthermore, recovering and detecting microbes such as Gram negative bacteria such as Salmonella and E. coli from some particular samples, such as spices, flavourings and herbs can be made more difficult due, at least in part, to the growth-inhibitory nature of some of the compounds that these samples possess. Current methods of recovering microbes from these sample types is typically performed by diluting the sample, sometimes significantly, to dilute out the undesired components of the sample, i.e., the growth inhibiting components of the spice/flavouring/herb sample such as alliums in garlic and onion, and phenolic compounds in the essential oils found in spices, such as eugenol in cloves (FIG. 17 shows some of the anti-microbial compounds found in some herbs and spices). For example, the FDA approved standard BAM method for the detection of Salmonella in cloves requires, for a 25 g sample, 25 litres of pre-enrichment broth, making the method impractical.

The below indicates the dilution factor required for compliance with the BAM reference method for some herbs and spices:
Black pepper, white pepper, celery seed or flakes, chili powder, cumin, paprika, parsley flakes, rosemary, sesame seed, thyme, and vegetable flakes
   Standard 1 in 10 enrichment with TSB
Onion flakes, onion powder, garlic flakes.
   Standard 1 in 10 enrichment with TSB containing 0.5% K2SO3
Allspice, cinnamon, cloves, and oregano.
   Require dilution of media to non-toxic levels typically at 1:100 to 1:1000 (for cloves)

Some reference methods, such as AFNOR and AOAC use potassium sulphite at a concentration of 5 g/L to neutralise the allicin present in onions and garlic.

Spices are notorious for their high frequency of microbial contamination and have been responsible for Salmonella outbreaks in the US and Europe. For example, bulk spices offered for import into the US have been found to be contaminated with Salmonella, for example 18% of ground coriander samples tested; 10% of oregano samples tested; and 6% of ground white pepper samples tested were found to be contaminated with Salmonella (Risk Profile: Pathogens and Filth in Spices (2017) Center for Food Safety and Applied Nutrition, Food and Drug Administration, U.S. Department of Health and Human Services).

Since spices are often added raw to season salads and cooked foods, the ability to detect Salmonella in spices is vital to preventing outbreaks of Salmonella.

The BAM standard method also requires a suitably high initial inoculum level of microbes so that sufficient microbial cells are present in the diluted sub-sample which is taken for recovery and testing. At least for this reason, current methods for recovering and detecting microbes such as Gram negative bacteria such as Salmonella and E. coli from spices, flavourings and herbs, are unreliable, inaccurate and are not suitable for routine high-throughput, rapid testing.

The BAM standard method for the detection of Salmonella requires an initial 24 hour incubation in liquid culture, followed by an at least 24 hour incubation on solid agar, which is then followed by further incubations on various media designed to identify certain biochemical characteristics which ultimately allows the presence of Salmonella cells to be determined. The BAM method is therefore unable to provide unequivocal results in a short period of time, with results not being obtained for at least several days.

Historically, single step culture methods resulted in poor recovery of Gram negative bacteria such as Salmonella as described in the seminal papers of Edel & Kampelmacher 1973 (Bull World Health Organ. 1973; 48(2): 167-174). In view of this it was widely accepted that additional cell culture steps are required in any diagnostic method with the aim of reviving such cells prior to further culture, plating and detection. Hence, pre-enrichment in non-selective culture media is an essential element of conventional methods (Stephens et al, 2000). For example, the detection of Salmonella often requires several stages of culture spread over as many as five days; enrichment steps are often included in the analysis to revive ‘sick’ bacteria and detection is often limited by the performance of such enrichment broths and cultures.

Thus, for the recovery of microorganisms such as Gram negative bacteria from clinical specimens, food, environmental test samples and other products that potentially harbour a heterogeneous population of bacteria, three general types of culture media are available: (1) non-selective media for primary isolation, (2) enrichment broths and (3) selective and/or differential agars.

The formulas for such media are generally complex and include ingredients that not only inhibit growth of certain bacterial species, i.e., they are selective, but also detect several biochemical characteristics that are important in making a preliminary identification of the micro-organisms present in the specimen, i.e., they are differentiating. In order to make rational selections, microbiologists must know the composition of each formula and the purpose and relative concentration of each chemical compound included. Unfortunately, the media available are often overly complex and the effect and amounts of the various components are generally little understood. Often the medium that is used is the same as that which has been used for several decades and may originally have been developed for an entirely different organism. For example, because of these inefficiencies, current detection rates of Salmonella are less than 50% within 15 days and 90% within 28 days (King, 2009).

GB2463369 discloses assay methods for detecting specific materials from microorganisms, and also compositions and methods for rapid growth of such microorganisms enabling detection of same.

However, there is still a need for new and better detection methods that enable the isolation and/or identification of GGram negative bacteria, such as Salmonella and E. coli, that are often found in very low numbers and in a heterogeneous microflora environment, with improved accuracy and speed. Further, any such methods should also be able to reliably detect Gram negative bacteria such as Salmonella and E. coli from a wide variety of sources such as cosmetics, different food products including frozen, lyophilised and liquid products, clinical samples such as urine, stool or blood samples and environmental samples.

There is a need for culture media that are well defined, do not contain surplus ingredients that may have little to no or even negative effects on the recovery of Gram negative bacteria such as Salmonella or E. coli and are optimal for the growth and rapid culture of even stressed microorganisms. Such culture media should negate the need for secondary/additional culture steps.

### SUMMARY

The present disclosure provides an improved methods, culture medium, compositions and kits for culturing Gram negative bacteria such as Salmonella cells or E. coli cells, particularly low abundance gram negative bacteria, such as low abundance Salmonella cells or E. coli cells, from a sample, for example from a sample comprising particulate matter, for example from a food sample. These cells are often stressed and difficult to recover in selective media, meaning that commonly a non-selective pre-culture step is used. Prior art methods often utilize compounds such as Brilliant Green (BG) to inhibit Gram positive bacteria, to selectively allow the Gram-negative Salmonella (and other Gram negative bacteria or other microorganisms) to grow to a detectable level. However, significant variability in the ability of these methods to recover target cells, such as Salmonella cells, particularly low abundant and/or stressed target or Salmonella cells make these methods less than ideal, particularly in view of the importance of early reliable detection of Salmonella contamination described above.

One disclosed method is considered to have utility in the selective culture of all gram negative cells from a sample. In some embodiments, selective culture includes the meaning of allowing the Gram negative bacterial species present in the sample to grow unhindered, or largely unhindered, whilst exerting a negative or a more significant negative growth effect on cells that are not gram negative, for example by restricting the growth of Gram positive cells present in the sample. Depending on the purpose of the culture, for example in the detection of Salmonella or E. coli, downstream detection steps can be tailored towards each target bacterial species, by, for example, using Salmonella or E. coli specific antibodies in an ELISA test. The disclosed culture methods are reliable and consistent to culture sufficient Gram negative bacteria from a sample so that the presence of each or a target species can later be identified, if desired. Accordingly, by target species we include the meaning of one or more Gram negative bacterial species that are to be identified or quantified, for example by downstream processing steps, such as identification or detection steps. A sample that is used with a method of culture described herein may comprise one or more target gram negative species.

By Gram negative bacteria, we include the meaning of any Gram negative species that may be present in a particular sample. The present disclosure has particular utility in the culture of Gram negative bacteria that are often difficult to recover from low abundance stressed states. Since the present disclosure provides a rapid and sensitive means of culture and detection of Gram negative bacteria, the present disclosure also has particular utility in the culture and subsequent detection of pathogenic Gram negative bacteria where early and reliable confirmation of infection, or the presence of a bacteria species in an environmental sample, is highly desired. For example and as discussed above, Salmonella contamination is a significant issue. The rapid and reliable detection of pathogenic strains of E. coli, for example those that produce the Shiga toxin, such as strain 0157. Accordingly, by Gram negative bacteria we include the meaning of Salmonella species and E. coli strains, such as the following E. coli strains: E. coli O104:H4, E. coli O157:H7, E. coli O145, E. coli O26, E. coli O111, E. coli O103, and E. coli O45.

Without wishing to be bound by any theory, the inventors consider that growth inhibitory dyes such as BG and Malachite Green (MG) bind to debris present in the sample, for example to food particles, to different degrees. This leads to differing amounts of free BG or MG for example available to inhibit growth of Gram-positive bacteria. This can have at least two consequences. In the first instance, if the food or other debris in the sample has a high ability to bind to BG or MG for example, the amount of free BG or MG may not be sufficient to allow suppression of Gram-positive bacteria, leading to any Gram negative cells, such as Salmonella cells or E. coli cells present in the sample being outcompeted by other microorganisms, leading to false negative sample. Conversely, if the food or other debris in the sample has a low ability to bind to BG or MG for example, there may be an excess of free BG or MG in the culture which whilst this is beneficial in terms of suppression of Gram-positive bacteria, can also be toxic to Gram negative cells such as Salmonella cells or E. coli cells, particularly stressed cells. In this instance it is considered that samples with a low abundance of Gram negative cells such as Salmonella cells or E. coli cells, and/or in which the cells are stressed cells are particularly susceptible to false negatives since the few cells present are killed or their growth is suppressed by the BG or MG.

Accordingly it is considered that the use of the Gram-positive inhibitory agents commonly used in methods of culturing Gram negative cells such as Salmonella cells or E. coli cells from samples such as food samples, clinical samples or swabs are not ideal, particularly in instances where they may be a high abundance of competing microflora and/or wherein the Gram negative bacteria such as Salmonella cells or E. coli cells present are present in a low abundance and/or are stressed cells.

The inventors have identified particular compounds defined by Formula I below, for example 4PYcq, which are effective against Gram-positive bacteria but which are not susceptible to this problem, and which allow the rapid culture of low abundant stressed cells to a detectable level in addition to this.

Accordingly, a first aspect of the disclosure provides a method for selectively culturing Gram negative bacteria including Salmonella or E. coli, for example for culturing Gram negative bacteria such as Salmonella or E. coli that may be present in a test sample, wherein the method comprises the step of culturing microorganisms present in the test sample in a culture medium, wherein the culture medium comprises an agent of Formula I

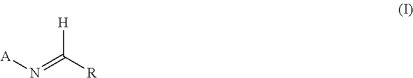

wherein A is selected from

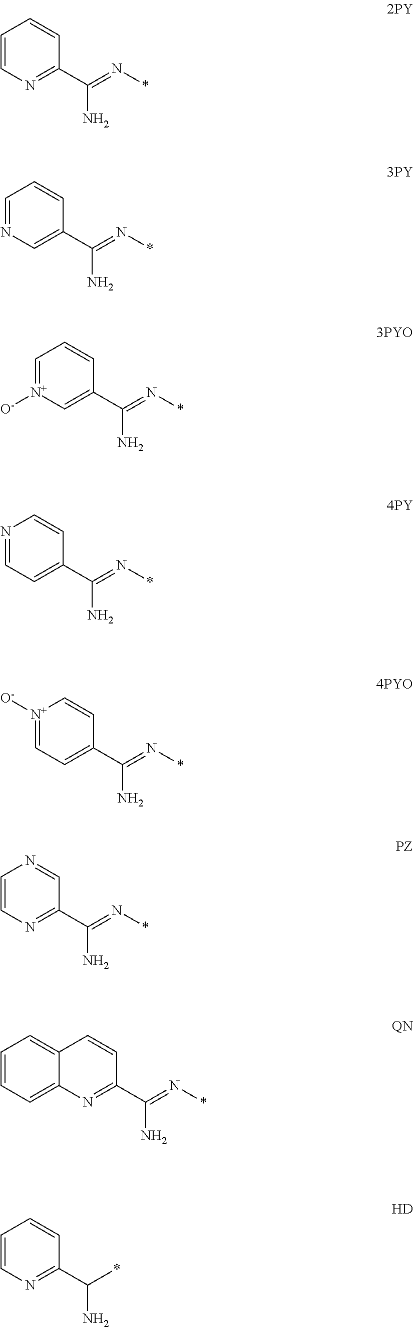

and R is selected from optionally substituted C₅₋₂₀ aryl, with the proviso that when A is 2PY, then R is not 1,3-dimethylphenyl.

In one embodiment, A is 2PY, 3PY, 4PY PZ QN or HD and R is selected from the group consisting of

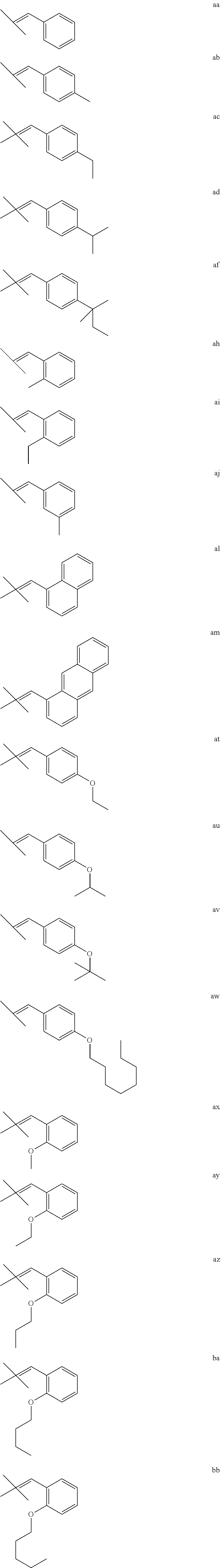

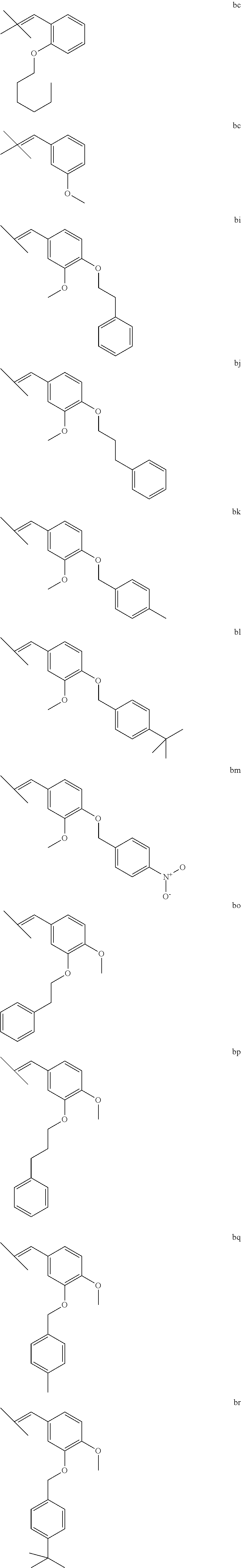

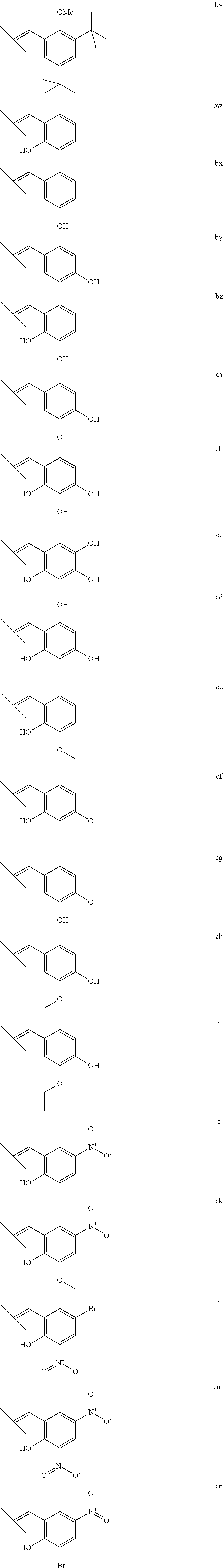

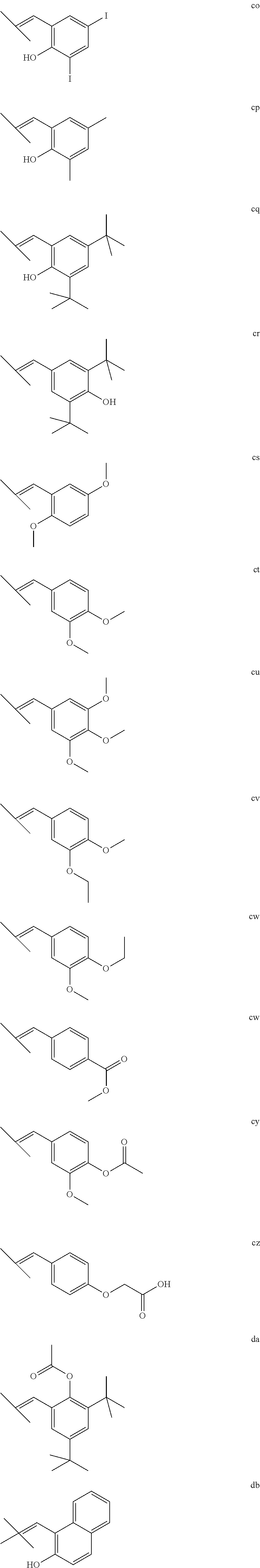

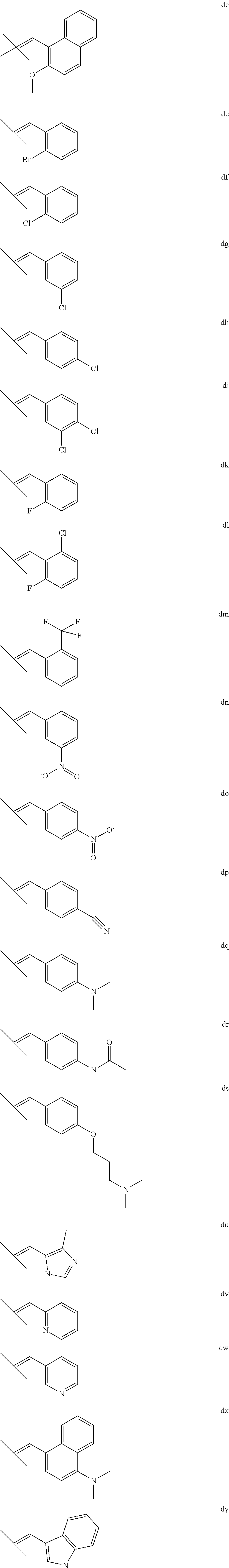

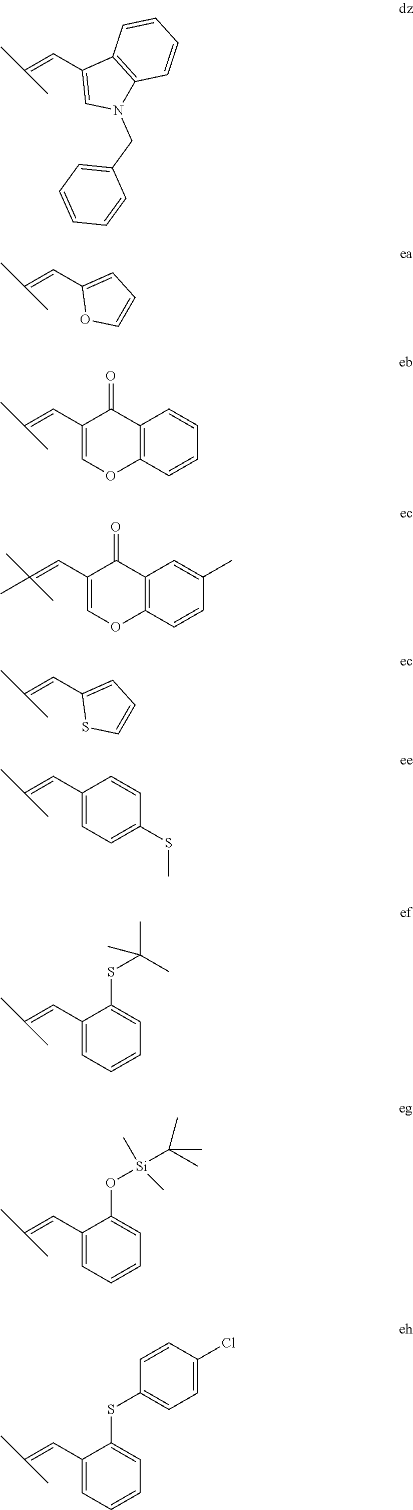

In a further embodiment the agent of Formula I is such that:

A is 2PY R is selected from at, ah, ai, aj, al or cj:

A is 3PY R is selected from af, ay, cc, cj or cl:

A is 4PY R is selected from af, am, cb, cc, cj co or cq;

A is HD R is selected from cd, ce, cf, cj or cl;

A is PZ R is selected from cb or cj; or

A is QN R is ca.

In yet a further embodiment, the agent of Formula I is selected from the group consisting of 3PYaf, 4PYaf, 4PYam, 4PYcb, 4PYco, 4PYcq, 4PYeh, HDcb, HDce, HDcf and HDdb.

In one embodiment, the agent of Formula I is 4PYcq.

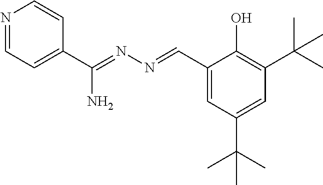 [SMILES: CC(C)(C)c1cc(/C=N/N=C(\N)c2ccncc2)c(O)c(C(C)(C)C)c1]

Further preferences for these compounds can be found in WO2006075159A1. The teachings of WO2006075159A1 which relate to these compounds are specifically incorporated by references, particularly pages 6 to 20 of the WO2006075159A1 published document.

Although the agents of Formula I have been described in WO2006075159A1 and were indicated to have a favourable activity against Gram positive bacteria versus Gram negative bacteria, the tests performed in the WO2006075159A1 were carried out on pure cultures of microorganisms. It is known that interactions between microorganisms in a heterogeneous microflora such as exists in for example a food sample or a probiotic sample are complex and the presence of just one different microorganism can influence the behaviour of another towards a particular compound. See for example Mohan et al 2015 RSC Advances 44, Angell et al 2006, Mikeskova et al 2012 Appl Microbio Biotechnol 95: 861-870, Wang et al 2017 PNAS 114: 10467-10472, and Parjis and Steenackers 2018 ISME 12: 2061-2075.

Prior to the present disclosure there was no reason to suppose that an agent of Formula I would even remain active in the presence of the various microorganisms that will be confronted during commercial bacterial testing such as Gram negative bacteria testing such as Salmonella or E. coli testing, or in the presence of a homogenised food matrix, for example. The inventors have surprisingly found that the agent of Formula I, for example 4PYcq, does remain active and appears to be uninfluenced by variation in the microflora environment. Further, the inventors have surprisingly found that the agent of Formula I is not influenced by the presence of debris in the culture, for example the presence of food particles.

Preferences for the agent of Formula I provided above apply to all aspects described herein.

In one embodiment, the agent of Formula I, for example 4PYcq, is present in the culture media in a concentration of:
a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L; and/or
b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L;
or in a range between any of the two above values.

In one embodiment the agent of Formula I is present in the culture media in a concentration of around 5 mg/L, optionally 5 mg/L; or is present in a culture media in a concentration of around 2.5 mg/L, optionally 2.5 mg/L.

It will be appreciated that the culture medium may also contain other anti-microbial agents to allow the further selective growth of the desired Gram negative cells, such as Salmonella cells or E. coli cells. For example, in one embodiment the culture medium further comprises one, two, three or four or more anti-bacterial agents, for example further comprises Cefsulodin and/or Novobiocin.

In the same or a different embodiment, the culture medium may also comprise ferric chloride and/or a sulphite compound, such as potassium sulphite or sodium sulphite. This embodiment is considered to be particularly useful, though not essential, for recovering Gram negative bacteria such as Salmonella or E. coli from food products that comprise spices, herbs and flavourings.

Accordingly, in one embodiment the disclosure provides a method for selectively culturing Gram negative bacteria such as Salmonella or E. coli that may be present in a test sample wherein the test sample is a spice, a herb or a flavouring.

It is considered that it is the sulphite component of potassium sulphite that inhibits the anti-microbial properties of allicin found in onions and garlic. Accordingly, by a sulphite compound we include the meaning of any sulphite compound, for example potassium sulphite and sodium sulphite (see J. Milk Food Technol. Vol. 39. No. 7, Pages 464-466 1976 Sulfite Compounds as Neutralizers of Spice Toxicity for Salmonella CLYDE R. WILSON and WALLACE H. ANDREWS). In an embodiment the sulphite compound is potassium sulphite.

Novobiocin is considered to be effective against Gram positive bacteria but has some toxicity towards Gram negative bacteria e.g. Proteus spp. and some coliforms. Cefsulodin is specifically effective against Pseudomonas aeruginosa. The culture medium may contain other such agents.

Unexpectedly, it is considered to be particularly beneficial to culture the microbial cells in the presence of both the agent of Formula I and Novobiocin, despite both agents being considered to target Gram positive bacteria.

In one embodiment, the culture medium comprises, or further comprises, for example in addition to an agent of Formula I and Novobiocin, an agent that inhibits the growth of, or kills, Pseudomonas aeruginosa, and/or other pseudomonads, for example comprises Cefsulodin. The concentration of Cefsulodin in the culture medium in one embodiment is:
between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L; or
8 mg/L.

As discussed above, in some embodiments the culture medium comprises novobiocin, for example comprises novobiocin at a concentration of:
between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
10 mg/L.

As discussed above, the culture medium may comprise ferric chloride. Also as discussed above, this is considered to be particularly useful in instances where the sample comprises spices, flavourings or herbs, but can be used in methods where the test sample does not comprise spices, flavourings or herbs. In one embodiment the concentration of ferric chloride in the culture medium is:
between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L.

In particular examples the concentration of ferric chloride in the media is between around 100 mg/L and 1.50 mg/L, for example between 1.10 mg/L and 1.40 mg/L; 1.20 mg/L and 1.30 mg/L. In further particular examples the concentration of ferric chloride in the media is around 1.33 mg/L, for example is 1.33 mg/L In another particular embodiment the concentration of ferric chloride in the media is 1.37 mg/L.

The skilled person will appreciate that the actual concentration of ferric chloride in the media can vary slightly from the quoted concentration of ferric chloride in the media, due to slight impurities in the stock ferric chloride. Accordingly, in one embodiment the above concentrations are actual concentrations of ferric chloride in the media. Accordingly, in one embodiment the actual concentration of ferric chloride in the media is 1.33 mg/L, achieved by providing culture media that comprises 1.37 mg/L ferric chloride that is 97% pure.

As discussed above, the culture medium may comprise potassium sulphite or sodium sulphite, as well as ferric chloride or instead of ferric chloride. As described above, this is considered to be particularly useful, but not essential, in instances where the sample comprises spices, flavourings or herbs, but can be used in methods where the test sample does not comprise spices, flavourings or herbs. In one embodiment the concentration of potassium sulphite in the culture medium is:
between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
at least 0.5 g/L, 0.75 g/L, 100 g/L, 1.25 g/L, 1.50 g/L, 1.75 g/L, 2.00 g/L, 225 g/L, 2.50 g/L, 2.75 g/L, 3.0 g/L, 3.5 g/L, or 4.0 g/L.

In particular examples the concentration of potassium sulphite in the media is between around 0.50 g/L and 1.50 g/L, for example between 0.75 g/L and 1.40 g/L; 1.00 mg/L and 1.30 g/L; 1.10 g/L and 1.20 g/L. In further particular examples the concentration of potassium sulphite in the media is around 1.00 g/L (0.1% w/v), for example is 1.00 g/L (0.1% w/v). In another particular embodiment the concentration of potassium sulphite in the media is 1.11 g/L.

The skilled person will appreciate that the actual concentration of potassium sulphite in the media can very slightly from the quoted concentration of potassium sulphite in the media, due to slight impurities in the stock potassium sulphite. Accordingly, in one embodiment the above concentrations are actual concentrations of potassium sulphite in the media. Accordingly, in one embodiment the actual concentration of potassium sulphite in the media is 1.00 g/L, achieved by providing culture media that comprises 1.11 g/L potassium sulphite that is 90% pure.

In one embodiment the culture medium comprises:
a) an agent of Formula I, for example at a concentration of
   a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L;
   and/or
   b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L;
   or in a range between any of the two above values;
   and/or
b) Cefsulodin at a concentration of:
   a) between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   b) at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L; or
   d) 8 mg/L and/or
c) Novobiocin at a concentration of
   a) between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   b) at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   d) 10 mg/L.

In one embodiment the culture medium comprises:
a) an agent of Formula I, for example at a concentration of
   a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L;
   and/or
   b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 70 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L;
   or in a range between any of the two above values;
   and/or
b) Cefsulodin at a concentration of:
   a) between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   b) at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L; or
   d) 8 mg/L and/or
c) Novobiocin at a concentration of
   a) between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   b) at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   d) 10 mg/L;
   and/or
d) Ferric chloride at a concentration of
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/L
   and/or
e) Potassium sulphite at a concentration of
   a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
   c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
   d) 100 g/L; or
   e) 1.11 g/L.

The test sample can be any sample which may comprise Gram negative bacterial cells, such as one or more Salmonella cells or one or more E. coli cells. The sample can also be a sample which is known to have no Gram negative bacterial cells, such as no Salmonella cells or no E. coli cells, or expected to have no Gram negative bacterial cells, as is often used as a negative control.

The test sample will typically be a sample that comprises or is expected to comprise at least one or more cells other than a Gram negative bacterial cell, for example other than a Salmonella cell or an E. coli cell, or is expected to comprise one or more other microorganism cells, for example a bacteria cell such as a Gram positive bacterial cell, and/or a fungal cell optionally a yeast cell. The present disclosure is considered to be particularly advantageous for use with such samples. In the same or different embodiment, the sample is a sample that is expected to comprise a low abundance of Gram negative bacteria, or a low abundance of a particular Gram negative target bacteria, such as a low abundance of Salmonella cells or a low abundance or E. coli cells.

In one embodiment the test sample comprises, or is expected to comprise a heterogeneous microflora environment. Accordingly, in one embodiment the test sample is not a pure culture of Gram negative bacteria, or not a pure culture of any one particular target Gram negative bacterial species, for example is not a pure culture of Salmonella or E. coli. The skilled person will understand what is meant by a heterogenous microflora environment, and typically implies an environment which comprises more than one species of microbe, for example the presence of more than one species of bacteria, or for example more than one class of microbe, for example may comprise bacteria and fungi.

In one embodiment, the sample is a sample that comprises, or is expected to comprise, a heterogeneous microflora environment and which also comprises a low abundance of total microbes, and/or a low abundance of target microbe, such as Salmonella or E. coli cells.

The test sample is, in some embodiments, typically any sample which may be expected to comprise one or more Gram negative cells. For such samples it is necessary to know whether a particular target Gram negative bacteria is present or not. Following culture using the methods of the present disclosure, the abundance of any Gram negative cells, such as Salmonella or E. coli cells will be increased, allowing downstream detection of the particular target species. The present disclosure is particularly advantageous in the culture of Gram negative bacteria from samples which comprise other non-microbial material, for example in samples that are not pure cultures of microorganisms but also include additional material such as food debris, cellular fluid such as mammalian or avian cellular fluid, cellular material that is not the cellular material of the microbial culture, for example cellular material includes saliva, biopsy samples, blood etc.

Accordingly, in one embodiment, the sample is a sample that comprises, or is expected to comprise:
a) a heterogeneous microflora environment;
b) a low abundance of total microbes, and/or a low abundance of target microbe, such as Salmonella or E. coli cells; and
c) non-microbial material, such as food debris, cellular fluid such as mammalian or avian cellular fluid, cellular material that is not the cellular material of the microbial culture, for example cellular material includes saliva, biopsy samples, blood etc.

The test sample may be:
a food product, for example meat, meat products including mince, eggs, cheese, milk, vegetables, chocolate, confectionery, peanut butter and the like including processed, dried, frozen or chilled food products, a spice, a herb or a flavouring product;
a clinical sample such as a biopsy sample, faecal, saliva, hydration fluid, nutrient fluid, blood, blood product, tissue extract;
a therapeutic product such as a vaccine, anaesthetic, pharmacologically active agent, imaging agent or urine sample, probiotics and the like;
a swab, for example a skin swab, a caecum swab, a faecal swab, a cloaca swab or a rectal swab: a swab of surfaces such as floors, food processing equipment, doors and walls; a swab of food products including animal carcass swabs;
a cosmetic sample such as foundation makeup, lip-balms, lotions, creams, shampoos and the like;
a sample of raw meat or raw poultry, or a swab taken from raw meat or raw poultry;
a sample of a liquid in which a sample such as food or a swab has been suspended, for example processed water such as water used to clean down equipment on the processing line (that could contain food particles) or water used to clean raw produce such as vegetables or meat, which again would contain food particulates; and/or
a sample that comprises non-microbial material.

By a food product we include the meaning of a food product for any organism, for example any mammal, for example a human. In one embodiment the food product is a human food product. In another embodiment the food product is a veterinary food product, for example a dog or a cat food product.

A spice is typically considered to be any aromatic vegetable substance in the whole, broken, or ground form, except for those substances which have been traditionally regarded as foods, such as onions, garlic and celery; whose significant function in food is seasoning rather than nutritional; that is true to name; and from which no portion of any volatile oil or other flavouring principle has been removed (FDA definition from Code of Federal Regulations, [Title 21, Volume 2, Revised as of Apr. 1, 2018-21—FOOD AND DRUGS; CHAPTER I—FOOD AND DRUG ADMINISTRATION; DEPARTMENT OF HEALTH AND HUMAN SERVICES; SUBCHAPTER B—FOOD FOR HUMAN CONSUMPTION; PART 101—FOOD LABELING; Subpart B-Specific Food Labeling Requirements).

Spices include the following non limiting examples:

Allspice, Anise, Basil, Bay leaves, Caraway seed, Cardamon, Celery seed, Chervil, Cinnamon, Cloves, Coriander, Cumin seed, Dill seed, Fennel seed, Fenugreek, Ginger, Horseradish, Mace, Marjoram, Mustard flour, Nutmeg, Oregano, Paprika, Parsley, Pepper, black; Pepper, white; Pepper, red; Rosemary, Saffron, Sage, Savory, Star anise, Tarragon, Thyme, Turmeric, and Paprika.

For example the term spice includes a seed, fruit, root, bark, or other plant substance primarily used for flavouring, colouring or preserving food.

A herb is typically considered to the leaves, flowers, or stems of plants used for flavouring or as a garnish.

A spice, a herb or a flavouring product includes, for example, packet mixes; seasoning mixes; dried herbs such as dried dill; fresh herbs; mixed herbs such as herbs de provence; dried individual spices such as cinnamon, dried peppercorns, sumac; mixed spices, such as Tandoori masala, garam masala, bahret seasoning; dried products such as dried onions and dried garlic; powders such as chilli powder, ginger powder, paprika powder or garlic powder; whole seeds such as cumin seeds, mustard seeds, fenugreek seeds, green cardamom pods or whole peppercorns; liquids, pastes or purees such as garlic paste or ginger puree.

In one embodiment a spice, herb or flavouring product is a product that comprises an amount of herb or spice that inhibits the growth of microbes such as Gram negative bacteria, such as Salmonella or E. coli, for example that inhibits the growth of microbes such as Gram negative bacteria, such as Salmonella or E. coli, by at least 5%, 10%, 15%, 20%, 25%, 30%, 35%, 40%, 45%, 50%, 55%, 60%, 65%, 70%, 75%, 80% or 90%, or inhibits the growth of microbes such as Gram negative bacteria, such as Salmonella or E. coli, between around 5% and 90%, between around 10% and 80%, 15% and 75%, 20% and 70%, 25% and 65%, 30% and 60%, 35% and 55%, 40% and 50%, around 45%.

In one embodiment the spice, herb or flavouring product inhibits the growth of such as Gram negative bacteria, such as Salmonella or E. coli, under standard culture conditions by at least 5%, 10%, 15%, 20%, 25%, 30%, 35%, 40%, 45%, 50%, 55%, 60%, 65%, 70%, 75%, 80% or 90%, or inhibits the growth of microbes such as Gram negative bacteria, such as Salmonella or E. coli between around 5% and 90%, between around 10% and 80%, 15% and 75%, 20% and 70%, 25% and 65%, 30% and 60%, 35% and 55%, 40% and 50%, around 45%.

In addition to spices, herbs, or flavourings that are considered to be “pure” spices or herbs, for example a packet that supposedly only contains cumin powder, for example, the spice, herb or flavouring product may be a food product to which the spice, herb or flavouring has already been added, for example may be a curry, for example a ready-made curry and may be in liquid form, for example. A curry may not be typically considered a “spice product” as defined above, however, it can be considered to be a “spicy product”, i.e. a product to which spices have been added. As discussed above, due to the large dilution factors required to dilute out the anti-microbial factors in “pure” spices, such as cloves or cumin, the ratio of the volume of the food product to the amount of spices, herbs, or flavourings that are added to the food products, such as curries, is not expected to be great enough to dilute out the anti-microbial factors that have been added to the food product in the spice, herb or flavouring. Accordingly, in one embodiment the test sample is a “spicy product”, or a “seasoned product”, i.e. is not considered to be largely made up of spices and/or herbs, but contains sufficient spices and/or herbs that any microbes present experience a growth inhibitory effect from the herbs and/or spices. Spicy products in some embodiments comprise food products such as ready-made curries, ready-made curry sauce. Accordingly, in one embodiment the sample is a food product that comprises one or more spices, herbs or flavourings.

In one embodiment the spice, herb or flavouring product comprises at least 10% w/w that is considered to be a spice, and/or a herb and/or a flavouring, for example at least 15%, 20%, 25%, 30%, 35%, 40%, 45%, 50%, 55% w/w, or at least 60% w/w, or at least 65% w/w, or at least 70% w/w, or at least 75% w/w, or at least 80% w/w, or at least 85% w/w, or at least 90% w/w, or at least 92% w/w, or at least 94% w/w, or at least 96% w/w, or at least 98% w/w, or at least 99% w/w or 100% w/w that is considered to be a spice and/or a herb and/or a flavouring. Since the addition of ferric chloride and potassium sulphite is considered to be beneficial in instances where the test sample is a food product that is a spice, a herb or a flavouring product, in one embodiment, where the test sample is not a food product that is a spice, a herb or a flavouring product, the culture medium does not comprise ferric chloride and/or potassium sulphite. However, without being bound by any theory, it is considered that there are no detrimental effects on the methods described herein where the test sample is not a spice, a herb or a flavouring product and where the media does comprise ferric chloride and potassium sulphite. Accordingly, the culture medium may comprise ferric chloride and/or potassium sulphite whether the test sample comprises a spice, herb or flavouring product, or does not comprise a spice, herb or flavouring product.

The test sample may be a whole sample, or may be a homogenised sample.

The sample may comprise any density of Gram negative cells, such as any density of Salmonella cells or E. coli cells. The sample may also comprise any density of other microbial cells such as competing microflora.

The sample may comprise what is considered to be a low density of Gram negative cells in general, or may be considered to have a low density of target Gram negative cells such as Salmonella or E. coli cells, for example less than 100 cfu (target cells such as gram negative bacteria in general, or Salmonella or E. coli)/25 g material. By cfu here we mean Gram negative cfu, or target cells cfu such as Salmonella cfu or E. coli cfu, i.e. a Gram negative cell, a Salmonella cell or an E. coli cell. For example, the sample may be expected to comprise a low density of Salmonella cells, but an overall high density of Gram negative bacterial cells.

By material we include the meaning of any type of sample, for example food matrices including spices and herbs, swab, clinical sample or cosmetic, as discussed above. For example the sample may comprise less than 90 cfu (target cells, such as gram negative bacteria in general, or for example Salmonella or E. coli)/25 g material, for example 80 cfu (target cells)/25 g material or less, for example 70 cfu (target cells)/25 g material or less, for example 60 cfu (target cells)/25 g material or less, for example 50 cfu (target cells)/25 g material or less, for example cfu (target cells)/25 g material or less, for example 30 cfu (target cells)/25 g material or less, for example 25 cfu (target cells)/25 g material or less, for example 20 cfu (target cells)/25 g material or less, for example 15 cfu (target cells)/25 g material or less, for example 10 cfu (target cells)/25 g material or less, for example 5 cfu (target cells)/25 g material or less, for example 4 cfu (target cells)/25 g material or less, for example 3 cfu (target cells)/25 g material or less, for example 2 cfu (target cells)/25 g material or less, for example 1 cfu (target cells)/25 g material or less. For example, the sample may comprise a lower density of Gram negative cells such as Salmonella cells or E. coli cells than 1 cfu (target cells)/25 g material, for example the sample may comprise 1 cfu (target cells)/50 g material, or for example a lower density of Gram negative cells such as Salmonella cells or E. coli cells such as 1 cfu (target cells)/100 g material, or for example a lower density of Gram negative cells such as Salmonella or E. coli cells than 1 cfu (target cells)/200 g material, for example a lower density of Gram negative cells such as Salmonella cells or E. coli cells than 1 cfu (target cells)/375 g material, or for example a lower density of Gram negative cells such as Salmonella cells or E. coli cells than 1 cfu (target cells)/500 g material.

Whether the sample is a liquid sample or a solid sample such as food or clinical or environmental swabs, once the sample is placed into the culture medium, the resultant culture, prior to culturing, may comprise between 1 cfu (target cells, such as gram negative bacteria in general, or for example Salmonella or E. coli)/10 ml culture media and 1 cfu (target cells)/1000 ml culture media, for example may comprise between 1 cfu (target cells)/20 ml culture media and 1 cfu (target cells)/900 ml culture media, 1 cfu (target cells)/30 nl culture media and 1 (target cells) cfu/800 ml culture media, 1 cfu (target cells)/40 ml culture media and 1 cfu (target cells)/700 ml culture media, 1 cfu (target cells)/50 ml culture media and 1 cfu (target cells)/600 ml culture media, 1 cfu (target cells)/60 ml culture media and 1 cfu (target cells)/500 ml culture media, 1 cfu (target cells)/70 ml culture media and 1 cfu (target cells)/400 ml culture media, 1 cfu (target cells)/80 ml culture media and 1 cfu (target cells)/300 ml culture media, 1 cfu (target cells)/90 ml culture media and 1 cfu (target cells)/200 ml culture media, for example 1 cfu (target cells)/100 ml culture media.

In an embodiment the culture is a liquid culture. However the disclosed methods, culture medium, and compositions are also considered to be appropriate for use with solid cultures such as agar cultures.

In an embodiment a culture method according to the disclosure allows the growth of stressed Gram negative cells, such as stressed Salmonella cells or stressed E. coli cells, and preferably has no or substantially no effect on the growth of stressed Gram negative cells, such as stressed Salmonella cells or stressed E. coli cells relative to the level of growth of the stressed Gram negative/Salmonella/E coli cells in non-selective media.

An appropriate density of the target Gram negative cells such as Salmonella or E. coli for use with the Salmonella detection methods of the disclosure is between 10⁴-10⁶ cfu/ml. Accordingly, in one embodiment the culture method results in a target cell density (such as Salmonella or E. coli) of at least 10⁴-10⁶ cfu/ml. This allows the cultured sample to be used directly in the detection method described herein without, for example, requiring centrifugation to further concentrate the cells.

The present culture method allows the rapid culture of target Gram negative species such as Salmonella or E. coli to detectable levels, for example via detection using the detection methods described herein. In one embodiment the present culture method results in a cell density of at least 10⁴-10⁶ cfu (target cells, such as gram negative bacteria in general, or for example Salmonella or E. coli)/ml following around 16 hours of culture, for example 20 hours of culture, for example results in a cell density of at least 10⁴-10⁶ cfu (target cells)/ml following around 16 hours of culture, for example 20 hours of culture from a low initial inoculum level, for example from less than 50 cfu (target cells)/225 ml, for example from less than 45 cfu (target cells)/225 ml, for example from less than 40 cfu (target cells)/225 ml, for example from less than cfu (target cells)/225 ml, for example from less than 30 cfu (target cells)/225 ml, for example from less than 25 cfu (target cells)/225 ml, for example from less than 20 cfu (target cells)/225 ml, for example from less than 15 cfu (target cells)/225 ml, for example from less than cfu (target cells)/225 ml, for example from less than 5 cfu (target cells)/225 ml, for example from less than 4 cfu (target cells)/225 ml, for example from less than 3 cfu (target cells)/225 ml, for example from less than 2 cfu (target cells)/225 ml for example 1 cfu (target cells)/225 ml.

The initial inoculum level that results in a cell density of at least 10⁴-10⁶ cfu (target cells)/ml following around 16 hours of culture, for example 20 hours of culture may be between 1 cfu (target cells)/10 ml culture media and 1 cfu (target cells)/1000 ml culture media, for example may comprise between 1 cfu (target cells)/20 ml culture media and 1 cfu (target cells)/900 ml culture media, 1 cfu (target cells)/30 ml culture media and 1 cfu (target cells)/800 ml culture media, 1 cfu (target cells)/40 ml culture media and 1 cfu (target cells)/700 ml culture media, 1 cfu (target cells)/50 ml culture media and 1 cfu (target cells)/600 ml culture media, 1 cfu (target cells)/60 ml culture media and 1 cfu (target cells)/500 ml culture media, 1 cfu (target cells)/70 ml culture media and 1 cfu (target cells)/400 ml culture media, 1 cfu (target cells)/80 ml culture media and 1 cfu (target cells)/300 ml culture media, 1 cfu (target cells)/90 ml culture media and 1 cfu (target cells)/200 ml culture media, for example 1 cfu (target cells)/100 ml culture media.

In one embodiment, the test sample is not a pure culture of Gram-negative bacteria or not a pure culture of any one particular Gram negative bacterial species, optionally not a pure culture of Salmonella or E. coli. Such pure cultures include cultures that comprise only, or substantially comprise only target bacterial cells. For example, in one embodiment a method according to the disclosure does not involve inoculating culture media with a culture of a single species of Gram-negative bacteria, such as Salmonella or E. coli.

An advantage of the present culture method is that it provides a culture comprising Gram negative bacteria such as Salmonella or E. coli at a detectable density, for example at a density of 10⁴-10⁶ cfu (target cells)/ml in a single step. Many of the prior art culture methods involve initial non-selective enrichment cultures to allow the Gram negative cells, such as Salmonella or E. coli cells to recover and begin growing. This not only adds a time-consuming further culture step, but makes the whole assay, from culture to detection, more complicated.

The culture method of the present disclosure in one embodiment comprises a single culture step, for example a single culture step at a single temperature.

In one embodiment the microorganisms present in the test sample are cultured in the culture medium at a temperature from 30° C. to 44° C., for example from 37° C. to 42° C., for example from 37.5° C. to 41.5° C., for example between 38° C. to 41° C., for example between 38.5° C. to 40.5° C., for example between 39° C. to 40° C. for example 39.5° C. In an embodiment the microorganisms are cultured at a temperature of 41.5° C., for example in single culture step.

In one embodiment of the culture method the microorganisms present in the test sample are cultured in a culture medium for between around 10-28 hours, for example between 12-26 hours, for example between 14-24 hours, for example between 16-22 hours, for example 18-hours, for example 16 hours or 20 hours.

In the same or another embodiment the microorganisms present in the test sample are cultured in the culture medium for less than 24 hours, for example less than 22 hours, for example less than 20 hours, for example less than 18 hours, for example 16 hours or less.

In other embodiments the microorganisms present in the test sample are cultured in the culture medium for 16 hours at a temperature from 30° C. to 44° C., for example from 37° C. to 42° C., for example from 37.5° C. to 41.5° C., for example between 38° C. to 41° C., for example between 38.5° C. to 40.5° C., for example between 39° C. to 40° C. for example 39.5° C., preferably 41.5° C.; or for 20 hours at a temperature from 30° C. to 44° C., for example from 37° C. to 42° C., for example from 37.5° C. to 41.5° C., for example between 38° C. to 41° C., for example between 38.5° C. to 40.5° C., for example between 39° C. to 40° C. for example 39.5° C., preferably 41.5° C. In one embodiment where the sample is an environmental sample the sample is cultured for 16 hours at a temperature from 30° C. to 44° C., for example from 37° C. to 42° C., for example from 37.5° C. to 41.5° C., for example between 38° C. to 41° C., for example between 38.5° C. to 40.5° C., for example between 39° C. to 40° C. for example 39.5° C., preferably at 41.5° C. In another embodiment were the sample is a food sample the sample is cultured for 20 hours at a temperature from 30° C. to 44° C., for example from 37° C. to 42° C., for example from 37.5° C. to 41.5° C., for example between 38° C. to 41° C., for example between 38.5° C. to 40.5° C., for example between 39° C. to 40° C. for example 39.5° C., preferably at 41.5° C. The skilled person will be able to determine the most suitable temperature and length of time for culture depending on the particular circumstances, for example food or sample type.

It will be appreciated that any suitable culture medium is appropriate for use with the present disclosure. The skilled person will understand which culture media are suitable for culturing Gram negative bacteria such as Salmonella cells or E. coli cells. In one embodiment the microorganisms present in the test sample are cultured in a nutrient culture medium comprising a base broth selected from the group consisting of peptone, tryptone, nutrient broth, L-broth, Gram negative broth, tryptic soy broth with yeast, modified tryptic soy broth and buffered peptone water.

In a particular embodiment the culture medium comprises buffered peptone water.

Base broths or basal media are basically simple media that support bacteria with minimal additional components. Generally such base broths simply need to provide a source of energy and maintain correct osmolarity, peptone, tryptone, nutrient broth (peptone, meat extract, optionally yeast extract and sodium chloride), L-broth (tryptone, yeast extract and sodium chloride), Gram negative broth, tryptic soy broth, tryptic soy broth with yeast and modified tryptic soy broth are suitable base components known in the art. Peptones are various water-soluble protein derivatives obtained by partial hydrolysis of a protein(s) by an acid or enzyme during digestion. Tryptic soy broth generally comprises tryptone (a pancreatic digest of casein), Soy peptone (a papaic digest of soybean meal) and sodium chloride, for example.

Modified tryptic soy broth may further comprise dextrose, bile salts and dipotassium phosphate. Particularly the base broth is selected from the group consisting of tryptone, nutrient broth, L-broth, Gram negative broth, peptone, tryptic soy broth, tryptic soy broth with yeast and modified tryptic soy broth. More particularly the base broth is selected from the group consisting of peptone, tryptic soy broth, tryptic soy broth with yeast modified tryptic soy broth and buffered peptone water. It is considered that Buffered Peptone Water may be particularly good for the recovery of stressed Salmonella.

Rappaport Vassiliadis Soya Peptone Broth (RVS) is used in the ISO Standard (6579-1:2017) for the culture and/or detection of Salmonella spp. The RVS broth may be useful in relation to some aspects of the disclosure, but in other embodiments the culture medium is not RVS broth. The RVS broth is formulated as follows:

Typical Formula g/litre

Soy Peptone 4.5

Sodium chloride 7.2

Potassium dihydrogen phosphate 1.26

Dipotassium hydrogen phosphate 0.18

Magnesium chloride anhydrous 13.58

Malachite green 0.033

In one embodiment it is preferred that the culture medium is not RVS. In another or the same embodiment it is preferred if the culture medium does not comprise magnesium ions and/or salts, for example magnesium chloride, and/or malachite green.

In another embodiment, the culture medium does not comprise any one of, any two or, or all of:
a) vancomycin;
b) Brilliant green; and
c) Malachite green.

The inventors have surprisingly found that vancomycin and novobiocin may react with one another under certain circumstances. For example in some circumstances, adding both vancomycin and novobiocin to the culture media results in a gelatinous precipitate. Accordingly, in one embodiment, where the culture medium comprises novobiocin, it does not comprise vancomycin.

In another embodiment, the culture medium does not comprise any one of, any two or more of, or all of:
a) bile salts, optionally deoxycholate;
b) Sodium thiosulphate;
c) Tergitol 4;
d) Sodium selenite;
e) magnesium chloride;
f) crystal violet;
g) bismuth ammonium citrate; and
h) acid fuschin.

For this and any other aspect of the disclosure, any particular preference for a particular feature from one aspect may be combined with any other particular preference for another feature in the same or a different aspect. For example, a method of culture according to the present disclosure may comprise:
a) culturing the microorganisms present in a dairy sample in a culture medium that comprises an agent of Formula I such as 4PYcq at a concentration of at least 5 ng/L; novobiocin at a concentration of between 8 mg/L and 20 mg/L; Cefsulodin at a concentration of between 6 mg/L and 15 mg/L; in a single culture step for 14-24 hours at 30° C.-44° C., and does not comprise BG.

Similarly, a method of culturing may comprise:
b) culturing the microorganisms present in a swab taken from a worktop/counter in a culture medium that comprises 5 mg/L 4PYcq; novobiocin at a concentration of 10 mg/L; Cefsulodin at a concentration of 8 mg/L; in a single culture step for 16 hours at 41.5° C. or for hours at 41.5° C., and optionally does not comprise BG, and or optionally does not comprise MG and/or optionally does not comprise vancomycin.

In one embodiment the culture medium does comprise vancomycin.

Since vancomycin is considered to have similar selective actions against Gram positive bacteria as the agent of formula I of the present disclosure, it is considered that the implements of the present disclosure may work well with vancomycin instead of or in addition to an agent of formula I. Accordingly, the disclosure also provides all aspects and embodiments disclosed herein but wherein the agent of formula I is replaced with vancomycin. However, vancomycin is currently considered to be a last-chance-antibiotic and its use is generally to be discouraged to prevent the emergence of resistant microorganisms, so the present methods and compositions and culture media that comprise an agent of formula I for example 4PYcq rather than vancomycin are considered to be advantageous. Accordingly, in some embodiments the culture media and methods involve the use of an agent of formula I, for example 4PYcq and do not involve the use of vancomycin.

It will be apparent from the above that the disclosure also provides a culture medium, for example a culture medium suitable for use in a method of aspect 1. Accordingly, in a second aspect the disclosure provides a culture medium suitable for use in a method of aspect 1. Preferences for features described in relation to the first aspect apply to the second aspect.

Accordingly, one embodiment provides a culture medium for the selective growth of Gram negative cells, for example for the growth of Salmonella cells or E. coli cells, for example the growth of Salmonella cells or E. coli cells that may be present in a test sample, wherein the culture medium comprises an agent of Formula I as defined in the first aspect of the disclosure, and at least one further agent.

In one embodiment the agent of Formula I is 4PYcq.

In another embodiment of the culture medium the agent of Formula I is present in the culture media in a concentration of:
a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L: and/or
b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 ng/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L;
or 5 mg/L.

In an embodiment the agent of Formula I is present in the culture media in a concentration of around 5 mg/L, for example 5 mg/L.

As described in relation to the first aspect of the disclosure, the culture medium of the disclosure may further comprise one, two, three or four or more anti-bacterial agents, for example may comprise Cefsulodin and/or Novobiocin.

In another embodiment the culture medium further comprises an agent that inhibits the growth of, or kills, Pseudomonas aeruginosa, and/or other pseudomonads, optionally comprises Cefsulodin.

In one embodiment the culture medium further comprises Cefsulodin at a concentration of
between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L;
optionally 8 mg/L.

In another embodiment the culture medium may comprise novobiocin in an amount of:
between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
10 mg/L.

The culture medium may comprise ferric chloride as described above, particularly in instances where the sample comprises spices, flavourings or herbs. In one embodiment the concentration of ferric chloride in the culture medium is:
a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
d) 1.33 mg/L; or
e) 1.37 mg/L.

The culture medium may comprise a sulphite compound such as potassium sulphite as described above, particularly in instances where the sample comprises spices, flavourings or herbs. In one embodiment the concentration of the sulphite compound such as potassium sulphite in the culture medium is:
a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
d) 1.00 g/L; or
e) 1.11 g/L.

In one embodiment, the culture medium does not comprise ferric chloride and/or potassium sulphite, for example does not comprise ferric chloride at a concentration of:
a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
d) 1.33 mg/L; or
e) 1.37 mg/L;
and/or does not comprise a sulphite compound such as potassium sulphite at a concentration of:
a) between around 0.5 g/L and 4 g/L, for example between 0.75 g/L and 3.5 g/L; 1.0 g/L and 3.0 g/L; 1.25 g/L and 2.75 g/L: 1.50 g/L and 2.50 g/L; 1.75 g/L and 2.25 g/L; or 2.00 g/L; and/or
b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
d) 100 g/L; or
e) 1.11 g/L.

In a particular embodiment the culture medium comprises novobiocin at a concentration of around 10 mg/L, for example 10 mg/L; and also comprises Cefsulodin at a concentration of around 8 mg/L, for example 8 mg/L.

In a further embodiment the culture medium comprises novobiocin at a concentration of around 10 mg/L, for example 10 mg/L; and comprises Cefsulodin at a concentration of around 8 mg/L, for example 8 mg/L; and comprises ferric chloride at a concentration of 1.33 mg/L; and comprises a sulphite compound such as potassium sulphite at a concentration of 1.00 g/L.

In one embodiment, the culture medium does not comprise an agent of Formula I for example 4PYcq, and does not comprise novobiocin and Cefsulodin. In this embodiment the culture medium comprises ferric chloride and/or a sulphite compound such as potassium sulphite, for example comprises ferric chloride at a concentration of:
a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
d) 1.33 mg/L; or
e) 1.37 mg/L; and/or
comprises a sulphite compound such as potassium sulphite at a concentration of
a) between around 0.5 g/L and 4 g/L, for example between 0.75 g/L and 3.5 g/L; 1.0 g/L and 3.0 g/L; 1.25 g/L and 2.75 g/L: 1.50 g/L and 2.50 g/L; 1.75 g/L and 2.25 g/L; or 2.00 g/L; and/or
b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
d) 1.00 g/L; or
e) 1.11 g/L.

Such a culture medium is considered to be useful since a culture medium according to this embodiment can be autoclaved and stored ready-made, into which the agent of Formula I, novobiocin and/or Cefsulodin can be added when required.

As for the first aspect of the disclosure, the base culture medium can be any suitable culture medium, of which the skilled person will be aware. In one embodiment the culture medium comprises a base broth selected from the group consisting of peptone, tryptone, nutrient broth, L-broth, Gram negative broth, tryptic soy broth with yeast, modified tryptic soy broth and buffered peptone water.

In a particular embodiment the culture medium comprises buffered peptone water.

In some embodiments a culture medium according to the disclosure does not comprise any one of, any two or, or all of:
a) vancomycin;
b) Brilliant green; and
c) Malachite green.

In some embodiments, where the culture medium comprises novobiocin, it does not comprise vancomycin.

The culture medium according to the disclosure may, in some embodiments, also not comprise any one of, any two or more of, or all of:
a) bile salts, optionally deoxycholate;
b) Sodium thiosulphate;
c) Tergitol 4;
d) Sodium selenite;
e) magnesium chloride;
f) crystal violet;
g) bismuth ammonium citrate; and
h) acid fuschin.

In some embodiments, it is preferred that the culture medium is not RVS or a culture medium comprising magnesium ions and/or salts, for example magnesium chloride, and/or malachite green.

In one embodiment the culture medium is a liquid culture medium.

In another embodiment the culture medium is a solid or gel culture medium, optionally comprises agar.

Again, as for the first aspect, any particular preference for a particular feature from one aspect may be combined with any other particular preference for another feature in the same or a different aspect. Accordingly in one embodiment the culture medium comprises a peptone base broth, 4PYcq, Cefsulodin and novobiocin. In another embodiment the culture medium comprises peptone buffered water, 4PYcq at a concentration of 5 mg/L, Cefsulodin at a concentration of 8 mg/L, and novobiocin at a concentration of 10 mg/L, and a further anti-Gram positive bacteria agent.

In a third aspect the disclosure provides a composition that is suitable for use in preparing a culture medium according to the second aspect of the disclosure.

Compositions according to the disclosure comprise an agent of Formula I as defined above, for example 4PYcq, and at least one further agent selected from the group comprising Cefsulodin and Novobiocin.

In an embodiment the compositions comprise Cefsulodin and Novobiocin.

In an embodiment the compositions comprise an agent of Formula I, for example 4PYcq; and Cefsulodin and Novobiocin.

In another embodiment, a composition according to the present disclosure comprises an agent of Formula I as defined above, for example 4PYcq, and at least one further agent selected from the group comprising Cefsulodin and Novobiocin; and also comprises ferric chloride and/or a sulphite compound such as potassium sulphite.

In another embodiment, which is considered to be particularly advantageous when the sample is a spice, a herb or a flavouring product, a spicy or seasoned product, or wherein the sample comprises one or more spices, herbs or flavourings, a composition comprises an agent of Formula I, for example 4PYcq; and Cefsulodin, Novobiocin, ferric chloride and a sulphite compound such as potassium sulphite.

In another embodiment the disclosure enables a composition that comprises, or consists essentially of, or consists of, ferric chloride and a sulphite compound such as potassium sulphite. For example, the disclosure enables a composition that comprises, or consists essentially of, or consists of, ferric chloride and a sulphite compound such as potassium sulphite, but which does not comprise any 1, 2 or all of an agent of formula I, for example 4PYcq; Cefsulodin; and Novobiocin.

In a further embodiment, the disclosure provides a composition that does not comprise ferric chloride and/or a sulphite compound such as potassium sulphite, for example does not comprise ferric chloride at a concentration of
a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L: 1.75 mg/L and 2.25 mg/L; or 2.00 mg/L; and/or
b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
d) 1.33 mg/L; or
e) 1.37 mg/L
and/or
   does not comprise a sulphite compound such as potassium sulphite at a concentration of
a) between around 0.5 g/L and 4 g/L, for example between 0.75 g/L and 3.5 g/L; 1.0 g/L and 3.0 g/L; 1.25 g/L and 2.75 g/L: 1.50 g/L and 2.50 g/L; 1.75 g/L and 2.25 g/L; or 2.00 g/L; and/or
b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
c) at least 0.5 g/L, 0.75 g/L, 1.00 g/L, 1.25 g/L, 1.50 g/L, 175 g/L, 2.00 g/L, 2.25 g/L, 2.50 g/L, 2.75 g/L, 3.0 g/L, 3.5 g/L, or 4.0 g/; or
d) 1.00 g/L; or
e) 1.11 g/L.

The skilled person will understand that often such compositions are supplied in a concentrated stock form, such that once the composition is added to the culture media, the correct working concentration is achieved.

In one embodiment the concentration of the agent of Formula I for example 4PYcq, and/or Novobiocin, and/or Cefsulodin, and/or ferric chloride, and/or a sulphite compound such as potassium sulphite is a concentrated concentration such that following dilution in for example the culture media, the resultant concentration of the:
i) agent of Formula I for example 4PYcq is:
   a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L; and/or
   b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L; ii) Novobiocin is:
   a) between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   b) at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   10 mg/L; and/or
iii) Cefsulodin is:
   a) between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   b) at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L; and/or
   d) Ferric chloride is
      a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
      b) is less than 4 mg/L, 3.5 mg/L, 3.0 mg/L, 2.75 mg/L, 2.50 mg/L, 2.25 mg/L, c 2.00 mg/L, 1.75 mg/L, 1.50 mg/L, 1.25 mg/L, 1.00 mg/L, 0.75 mg/L, 0.50 mg/L or 0.25 mg/L; and/or
      c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
      d) 1.33 mg/L; or
      e) 1.37 mg/L and/or
   e) a sulphite compound such as Potassium sulphite is
      a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
      b) less than 4.00 g/L, 3.5 g/L, 3.0 g/L, 275 g/L, 2.50 g/L, 2.25 g/L, 2.00 g/L, 1.75 g/L, 1.50 g/L, 1.25 g/L, 1.00 g/L, 0.75 g/L, or 0.50 g/L: and/or
      c) at least 0.5 g/L, 0.75 g/L, 1.00 g/L, 1.25 g/L, 1.50 g/L, 1.75 g/L, 200 g/L, 2.25 g/L, 2.50 g/L, 2.75 g/L, 3.0 g/L, 3.5 g/L, or 4.0 g/L; or
      d) 1.00 g/L; or
      e) 1.11 g/L.

In one embodiment of a composition according to the disclosure the concentration of:
a) the agent of Formula I is between 1.00 mg/ml and 1.50 mg/ml, for example between 1.10 mg/ml and 1.40 mg/ml, for example between 1.20 mg/ml and 1.30 mg/ml, optionally wherein the concentration is 1.125 mg/ml, for example wherein the agent of Formula I is dissolved in 70% v:v ethanol;
b) the Novobiocin is between 2.00 mg/ml and 3.00 mg/ml, for example between 2.10 mg/ml and 2.90 mg/ml, for example between 2.20 mg/ml and 2.80 mg/ml, for example between 2.30 mg/ml and 2.70 mg/ml, for example between 2.40 mg/ml and 2.60 mg/ml, for example 2.50 mg/ml, for example the concentration may be 2.7 mg/ml or 2.25 mg/ml, preferably 2.25 mg/ml, for example wherein the novobiocin is dissolved in 70% v/v ethanol
c) the Cefsulodin is between 1.4 mg/ml and 2.0 mg/ml, for example between 1.5 mg/ml and 1.9 mg/ml, for example between 1.6 mg/ml and 1.8 mg/ml, for example between 1.7 mg/ml and 1.7 mg/ml, optionally wherein the concentration is 1.8 mg/ml, optionally wherein the Cefsulodin is dissolved in 70% v/v ethanol
d) Ferric chloride is between around 10 mg/L to 10 g/L, for example between 100 mg/L and 5 g/L, 1 g/L and 3 g/L;
   and/or
e) a sulphite compound such as Potassium sulphite is between around 5 g/L and 50 g/L, for example between 10 g/L and 40 g/L, or 20 g/L and 30 g/L.

In one embodiment the disclosure enables two compositions, wherein a first composition comprises an agent of formula I, novobiocin and Cefsulodin; and wherein the second composition comprises ferric chloride and a sulphite compound such as potassium sulphite.

In another embodiment, the disclosure enables a composition that comprises an agent of formula I, novobiocin and Cefsulodin; and also provides a culture medium wherein the culture medium comprises ferric chloride and a sulphite compound such as potassium sulphite. Preferences for the concentrations of each agent are as discussed above.

The composition or compositions according to the disclosure may be supplied in any form, for example as a liquid or powder. In an embodiment the compositions are lyophilised. The inventors have identified surprising benefits associated with the lyophilised form of the composition, particularly wherein the compositions comprise the agent of formula I for example 4PYcq, Novobiocin and Cefsulodin.

In one embodiment the lyophilised composition comprises the agent of Formula I for example 4PYcq, Novobiocin and Cefsulodin in a ratio of C-X:Novo:Cef of 1:2:1.6 ratio, for example the compositions may comprise 5 mg of the agent of Formula I for example 4PYcq with 10 mg of Novobiocin with 8 mg of Cefsulodin, for example in a lyophilised composition.

As discussed above, any particular preference for a particular feature from one aspect may be combined with any other particular preference for another feature in the same or a different aspect.

The improved culture methods allow the reliable and rapid culture of Gram negative bacteria, such as Salmonella or E. coli, to detectable levels, even when the cells are initially in low abundance and/or are in a stressed state. Accordingly, a fourth aspect of the disclosure provides a method for quantifying or detecting the presence or absence of Gram negative bacteria such as Salmonella or E. coli in a test sample, or for example detecting the presence or absence of a target Gram negative bacteria such as Salmonella or E. coli in a test sample, wherein a method comprises selectively culturing microbial cells present in the test sample according to the first aspect of the disclosure and subsequently detecting the presence or absence of the Gram negative bacteria, for example detecting the presence or absence of the target Gram negative bacteria, for example detecting the presence or absence of Salmonella or E. coli cells.

The skilled person will understand that detecting the presence or absence of a target Gram negative species such as Salmonella or E. coli can be carried out by any suitable means, including for example PCR or amplification based assays in which for example Salmonella or E. coli specific nucleic acids are amplified and detected, northern blot, western blot, immunofluorescence, flow cytometry and ELISA methods. It is considered that the culture method described herein reliably produces enough target Gram negative bacterial cells such as Salmonella or E. coli cells from a positive sample to allow sensitive detection by any of these methods.

Prior art methods that involve for example BG or malachite green are considered by the inventors to be an unreliable selective agent for primary selective enrichment of Gram negative cells such as Salmonella and E. coli, particularly from, for example, a low abundance sample of stressed or unstressed Salmonella or E. coli cells and/or from a food sample containing competing microflora, and is unable to allow sensitive detection, for example sensitive detection by antibody based methods such as ELISA based methods.

The assay method may be direct or indirect. In a direct binding or non-competitive assay (direct or indirect), also referred to as a ‘sandwich assay’, core oligosaccharides are bound to a surface and a binding member, such as an antibody, is reacted with any core oligosaccharides of the microorganism of interest.

In a competitive assay, the core oligosaccharide in the test sample competes with labelled core oligosaccharide for binding to a binding member. The amount of labelled binding member bound to the core oligosaccharide is then measured. In this method, the response will be inversely proportional to the concentration of core oligosaccharide in the sample. This is because the greater the response, the less core oligosaccharide in the ‘unknown’ or test sample was available to compete with the labelled core oligosaccharide.

It is considered that a direct binding or non-competitive assay may be more reliable across a variety of food sample types than a competitive assay.

The test sample is exposed to at least one binding member which has binding specificity to a core oligosaccharide of the microorganism of interest.

The test sample may be any test sample as discussed above. In one embodiment, and as described previously, the test sample may be:
a food product, optionally meat, meat products including mince, eggs, cheese, milk, vegetables, chocolate, confectionery, peanut butter and the like including processed, dried, frozen or chilled food products, a spice, a herb or a flavouring product;
a clinical sample such as a biopsy sample, faecal, saliva, hydration fluid, nutrient fluid, blood, blood product, tissue extract, vaccine, anaesthetic, pharmacologically active agent, imaging agent or urine sample and the like;
a swab, for example a skin swab, a caecum swab, a faecal swab, a cloaca swab or a rectal swab; a swab of surfaces such as floors, doors and walls for example in a food processing factory; a swab of food products including animal carcass swabs;
a cosmetic sample such as foundation makeup, lip-balms, lotions, creams, shampoos and the like;
a sample of a liquid in which a sample such as food or a swab has been suspended;
a sample of raw meat or raw poultry, or a swab taken from raw meat or raw poultry.

Typically, the test sample is treated to sufficiently release one or more core oligosaccharides for example from LPS from any microorganism present within the test sample. However, the assay is also considered to work without the treatment of the microorganisms to release one or more core oligosaccharides, since the capture and/or binding members described herein are considered to be able to bind to the core oligosaccharides and/or LPS without such treatment. Accordingly, such treatment steps are optional.

In some embodiments, the test sample may be treated in any way suitable to cause release of bacterial lipopolysaccharide (LPS) and or core oligosaccharide from the cell membrane of a microorganism. Methods of treating the test sample are described in GB 2463369 B.

In one embodiment of a method of quantifying or detecting the presence or absence of a target Gram negative bacteria such as Salmonella or E. coli in a test sample, the cultured microbial cells are treated so as to cause release of bacterial LPS and/or core oligosaccharide from any microorganism present within the test sample. This skilled person will understand how this can be achieved. For example, in one embodiment said treating comprises heating the microbial cells, for example heating to about 60° C. to about 100° C., for example 65° C. to 100° C., for example 70° C. to 100° C., for example 75° C. to 100° C., for example 80° C. to 100° C., for example 85° C. to 100° C., for example 90° C. to 100° C., for example 95° C. to 100° C. In one embodiment the cultured microbial cells are heated for about 30 seconds to about 30 minutes, for example about 2, 3, 4, 5, 6, 7, 8, 9, 10, 11, 12, 13, 14, 15, 16, 17, 18, 19, 20, 21, 22, 23, 24 or about 25 minutes, for example 15 to 20 minutes.

In another embodiment, the microbial cells (for example in the culture media) is heated to a rolling boil in a water bath for 20 min at 90-100° C.

In a particular embodiment, the microbial cells (for example in the culture media) are heated to 15 to 20 minutes at between 85° C. to 100° C.

The heating of the culture medium is considered to enhance the detection of Salmonella.

Lipopolysaccharides (LPS)

LPSs are an essential component of all Gram-negative and some Gram-positive bacterial outer membranes. They are believed to be the principle agents responsible for inflammatory responses in patients infected with such bacteria. Examples of Gram-negative bacteria include Escherichia coli, Salmonella, Shigella and Campylobacter. Listeria is a Gram-positive bacterium.

Most of the characterized LPSs have the same principal structure; the structure of the LPS has been determined as consisting of three distinct regions: a lipid A region, a core oligosaccharide and an o-polysaccharide chain (FIG. 1). This structure is especially conserved in the lipid A and inner core parts of the LPS. Because of this structural conservation, binding members, such as antibodies, to the lipid A region may not be specific to a particular species leading to false positives in any molecular detection steps. Further, the use of multiple binding members to, for example, the core region is unsatisfactory since such binding members may compete for the same epitope or, because of the close proximity of epitopes, may hinder each other's respective binding reaction. Thus, detection methods of the prior art have relied on binding members specific to the cell surface or flagellae of, for example, Salmonella, since these are easily accessible.

LPSs are generally isolated from bacteria by aqueous phenol extraction followed by purification. Isolated LPSs can then be characterised by, for example, SDS-PAGE, mass spectrometry and NMR (Raetz, 1996). The inventors have discovered that the core oligosaccharide region may be released or made accessible or available for detection, for example by antibody binding techniques, through use of a rapid method utilising a detergent and the application of heat. Use of such a simple methodology would not be suitable for detection of, for example, cell surface antigens or flagellae because detergents are known to interact with lipids and would destroy or disrupt lipid A epitopes with which binding members may react. Whilst detergent alone could be used, the use of heat is further advantageous since it breaks down the LPS into detectable monomers and has the added advantage of killing pathogenic bacteria.

In some embodiments, once the cells have been treated, methods according to the disclosure comprise exposing the cultured microbial cells or the bacterial LPS and/or core oligosaccharide of the treated cultured microbial cells to a capture member, resulting in captured cells or captured bacterial LPS and/or core oligosaccharide, for example this step may be performed after the cultured microbial cells are treated so as to cause release of bacterial LPS and/or core oligosaccharide from any microorganism present within the test sample.

In one embodiment the capture member is an antibody or antibody fragment, optionally a biotinylated antibody or antibody fragment, for example the capture member may be a sheep polyclonal antibody raised against Salmonella, or may be a monoclonal antibody, for example the capture member may be any of the 3D11, G7, M181 or T6 antibodies.

In an embodiment the capture member is immobilised to a substrate or solid surface, for example immobilised to a substrate or solid surface via an intermediate binding partner, for example wherein the intermediate binding partner is a linkage comprising biotin and streptavidin. This results in immobilisation of the cultured microbial cells or the bacterial LPS and/or core oligosaccharide to the surface.

In other embodiments, for example a competitive assay, the test sample is applied to or contacted by a surface on which is already immobilised a known or standard quantity of core oligosaccharide, LPS or monomer. Core oligosaccharide, LPS or monomer from both the known or standard compete with core oligosaccharide, LPS or monomer from the test sample for binding to the at least one binding member. Core oligosaccharides, LPSs or monomers may be directly immobilised to said surface, for example, by way of non-covalent hydrophobic interactions or indirectly as described above.

The test sample should be exposed to at least one capture member for a sufficient time to allow for the cultured microbial cells or the bacterial LPS and/or core oligosaccharide to bind to the at least one binding member to form a complex, for example a core oligosaccharide/capture member complex. Suitable times include from about 1 minute to about 4 hours, particularly from about 30 minutes to about 2 hours, particularly about 45 minutes, 1 hour and 1.5 hours.

In one embodiment, the at least one capture binding member is immobilised to a substrate or surface via an intermediate binding partner, such as an antibody, conjugate or other linkage.

In one embodiment, the intermediate binding partner is a linkage comprising biotin and streptavidin.

In an embodiment, the first capture member is conjugated with the vitamin biotin, and is then bound to a streptavidin plate.

The inventors have found that the use of an intermediate binding partner gives more effective presentation of the capture binding member, for example antibody, resulting in better analytical sensitivity of up to a log e.g. 10⁵ CFU/ml to 10⁴ CFU/ml. In addition, this format allows smaller amounts of antibody to be used and gives characteristically low background signals.

The surface or substrate to which the core oligosaccharide(s), LPS and/or other microbial fragments are bound (for example via a first capture member and intermediate binding partner) may be of a material known in the art, for example, organic polymers such as plastics, glasses, ceramics and the like. Particular organic polymers include polystyrene, polycarbonate, polypropylene, polyethylene, cellulose and nitrocellulose. A suitable polymer may be polystyrene and more particularly gamma-irradiated polystyrene. The surface itself may be in the form, or part, of a sheet, microplate or microtitre plate, tray, membrane, well, pellet, rod, stick, tube, bead or the like.

In one embodiment, the test sample can be assayed in a lateral flow immunoassay test.

In an embodiment, the cultured microbial cells or the bacterial LPS and/or core oligosaccharide of the treated cultured microbial cells are immobilised onto a surface through use of an intermediate binding member, such as an antibody, conjugate or other linkage, for example as indicated above.

In one embodiment the capture member is specific for microorganisms, for example specific for bacteria, for example specific for Gram negative bacteria. Preferably the capture member has specificity for Salmonella or E. coli.

In one embodiment the capture member is selected from the group consisting of:
a monoclonal antibody with specificity for the LPS or core oligosaccharide of Salmonella, optionally any of the 3D11, G7, M181 or T6 antibodies:
ViroStat product number:
   Salmonella sp. 6371-6394, 6301-6347, 6301-flagella, 6321-flagella, 6331-typhimurium, 6392-common core, 6393, 6394, 6391, 6381, 6371;
   Salmonella paratyphi A 6347-LPS; or
   Salmonella typhi 6301, 6321, 6345, 6331, 6347.

In a further embodiment of one method according to the disclosure the captured cells or captured bacterial LPS and/or core oligosaccharide are exposed to a detection member, for example in some embodiments said exposing to a detection member occurs after the captured cells or captured bacterial LPS/and/or core oligosaccharide is exposed to a wash solution. The skilled person will understand the need to wash any unbound capture member from the sample. In one embodiment the wash solution comprises:
a) monovalent ions wherein the molar concentration of monovalent ions and/or salts 30 in the wash solution is at least 100, 137, 150, 200, 250, 300, 350, 400, 450, 500, 550, 600 or 650 mM, or between 100 and 700 mM or 750 mM or 800 mM, or between 300 and 650 mM, optionally wherein the molar concentration of monovalent ions and/or salts present in the wash solution is at least 200 mM, 300 mM, 400 mM, 500 mM, 600 mM, 700 or 800 mM optionally 471 mM, 513.3 mM or 599 mM.
optionally wherein the monovalent ion and/or salt in the wash solution is a lithium, sodium or potassium ion and/or salt, optionally wherein the monovalent salt is LiCl, NaCl and/or KCl:
   or
b) divalent ions and/or salts wherein the molar concentration of divalent ions and/or salts in the wash solution is between 1 and 30 mM, optionally between 5 and 15 mM, between 12 and 14 mM or between 5 and 8 mM, optionally 6 to 12 mM, optionally 8.4 mM, 6.6 mM, 13 mM or 26 mM,
optionally wherein the divalent ion and/or salt in the wash solution is a magnesium or calcium ion and/or salt, optionally wherein the divalent salt is MgCl₂ and/or CaCl₂.

In one embodiment, the wash buffer may comprise Tris buffer with additional monovalent ions and/or salts added to arrive at the above molar concentration.

The wash buffer may comprise a detergent, for example, Tween® 20, for example between 1 and 10 mM, for example 3-5 mM, for example 4 mM.

In one embodiment, the molar concentration of monovalent ions and/or salts in the wash solution is between 700 and 800 mM, for example 750 mM.

In a further embodiment, the molar concentration of monovalent ions and/or in the wash solution is between 550 and 650 mM, for example 599 mM.

In one embodiment, the molar concentration of monovalent ions and/or salts in the wash solution is between 500 and 550 mM, for example 513.3 mM.

In one embodiment, the molar concentration of monovalent ions and/or salts in the wash solution is between 450 and 480 mM, for example 471 mM.

In one embodiment, the monovalent ions and/or salts in the wash solution are lithium, sodium or potassium ions and/or salts.

In one embodiment, the monovalent salt present in the wash solution is NaCl, KCl and/or LiCl.

In one embodiment, the test sample and at least one second binding member are exposed to a wash solution wherein the molar concentration of NaCl, KCl and/or LiCl is at least 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM 675 mM, 700 mM, 725 mM, 750 mM, 775 mM, 800 mM, 825 mM or 850 mM.

In one embodiment, the molar concentration of NaCl, KCl and/or LiCl in the wash solution is between 700 and 800 mM, for example 750 mM.

In a further embodiment, the molar concentration of NaCl, KCl and/or LiCl in the wash solution is between 550 and 650 mM, for example 599 mM (3.5% NaCl).

In one embodiment, the molar concentration of NaCl, KCl and/or LiCl in the wash solution is between 500 and 550 mM, for example 513.3 mM (3% NaCl).

In one embodiment, the molar concentration of NaCl, KCl and/or LiCl in the wash solution is between 450 and 480 mM, for example 471 mM (2.75% NaCl).

Wash solutions comprising molar concentrations of NaCl, KCl and/or LiCl between 471 mM and 599 mM, for example 513.3 mM, are suitable for reducing the frequency of false positive signals, for example, arising from poultry samples (as shown in Example 5).

In one embodiment, the molar concentration of LiCl, NaCl and/or KCl between 450 and 480 mM, for example 471 mM, in the wash buffer is suitable for detecting target Gram negative species such as Salmonella or E. coli in a test sample when the detection member is a mouse monoclonal antibody with specificity for the core oligosaccharide, such as antibodies designated 3D11, G7, M181, T6 or similar commercially available monoclonal antibodies with specificity for the core oligosaccharide of Salmonella; or antibodies with specificity for E. coli.

In one embodiment, the test sample and at least one detection member are exposed to a wash solution of divalent ions and/or salts, wherein the molar concentration of divalent ions and/or salts is between 1 and 30 mM, for example between 5 and 15 mM, for example between 12 and 14 mM or between 5 and 8 mM.

In one embodiment, the wash buffer may comprise Tris buffer with divalent ions and/or salts added.

The wash buffer may comprise a detergent, for example, Tween® 20, for example between 1 and 10 mM, for example 3-5 mM, for example 4 mM.

In one embodiment, the molar concentration of divalent ions and/or salts present in the wash solution is between 20 mM and 30 mM, for example 26 mM (0.25% MgCl₂).

In one embodiment, the molar concentration of divalent ions and/or salts present in the wash solution is between 10 mM and 20 mM, for example 13 mM (0.12% MgCl₂).

In one embodiment, the molar concentration of divalent ions and/or salts present in the wash solution is between 5 mM and 10 mM, for example 6.6 mM (0.062% MgCl₂).

In one embodiment, the molar concentration of divalent ions and/or salts present in the wash solution is between 7 mM and 10 mM, for example 8.4 mM (0.08% MgCl₂).

In one embodiment, the divalent ion/salt in the wash solution is a magnesium or calcium ion/salt.

In another embodiment, the divalent salt is MgCl₂ and/or CaCl₂).

In one embodiment, the test sample and detection member are exposed to a wash solution of MgCl₂ and/or CaCl₂, wherein the molar concentration of MgCl₂ and/or CaCl₂ is between 1 and 30 mM, for example between 5 and 15 mM, for example between 12 and 14 mM or between and 8 mM.

In one embodiment, the molar concentration of MgCl₂ and/or CaCl₂ in the wash solution is between 20 mM and 30 mM, for example 26 mM (0.25% MgCl₂).

In one embodiment, the molar concentration of MgCl₂ and/or CaCl₂ in the wash solution is between 10 mM and 20 mM, for example 13 mM (0.12% MgCl₂).

In one embodiment, the molar concentration of MgCl₂ and/or CaCl₂ in the wash solution is between 5 mM and 10 mM, for example 6.6 mM (0.062% MgCl₂).

In one embodiment, the molar concentration of MgCl₂ and/or CaCl₂ in the wash solution is between 7 mM and 10 mM, for example 8.4 mM (0.08% MgCl₂).

In one embodiment, the molar concentration of MgCl₂ and/or CaCl₂ at 26 mM, 13 mM, 6.6 mM and 8.4 mM, in the wash buffer is suitable for detecting target Gram negative species, such as Salmonella or E. coli in a test sample when the detection member is a mouse monoclonal antibody with specificity for the core oligosaccharide, such as antibodies designated 3D11, G7, M181, T6 or similar commercially available monoclonal antibodies with specificity for the core oligosaccharide of Salmonella; or antibodies with specificity for E. coli.

This aspect of the disclosure reflects the inventors' surprising observation that the frequency of false positive results can be reduced by:
a) increasing levels of monovalent ions and/or salts present in the wash buffer; or
b) the presence of low levels of divalent ions and/or salts present in the wash buffer.

As discussed above, the captured cells or bacterial LPS and/or core oligosaccharide should be exposed to the detection member for long enough for the detection member to associate with the captured complex. For example suitable times include from about 1 minute to about 4 hours, particularly from about 30 minutes to about 2 hours, particularly about 45 minutes, 1 hour and 1.5 hours.

Once the detection member binds to the captured cells or captured bacterial LPS and/or core oligosaccharide a detection complex is formed.

In some embodiments the detection member is an antibody or antibody fragment.

In some embodiments the detection member is specific for microorganisms, for example specific for bacteria, for example specific for Gram negative bacteria. Preferably the detection member has specificity for Salmonella or E. coli.

In some embodiments the detection member is an antibody selected from the group consisting of:
a monoclonal antibody with specificity for the LPS or core oligosaccharide of Salmonella, optionally any of the 3D11, G7, M181 or T6 antibodies
ViroStat product number:
   Salmonella sp. 6371-6394, 6301-6347, 6301-flagella, 6321-flagella, 6331-typhimurium, 6392-common core, 6393, 6394, 6391, 6381, 6371;
   Salmonella paratyphi A 6347-LPS; or
   Salmonella typhi 6301, 6321, 6345, 6331, 6347.

Whilst the skilled person will appreciate that antibodies tend to function optimally at physiological salt concentrations, the present inventors have surprisingly found a ten-fold increase in sensitivity when the sample is exposed to the detection member at surprisingly low salt concentrations, concentrations that are beyond those that the skilled person would routinely test as part of assay optimisation. For example, in one embodiment said exposing to said detection member is performed at a molar concentration of monovalent ions and/or salts that is:
a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
optionally wherein the monovalent ion and/or salt is a lithium, sodium and/or potassium ion and/or salt, optionally wherein the monovalent salt is LiCl, NaCl and/or KCl.

In one embodiment, the resultant molar concentration of monovalent ions and/or salts during the exposure of the captured test sample to the detection member is no more than 200 mM, for example less than 150 or 137 or 120 or 100 or 50 or 40 mM, for example between 20 and 120 mM or between 20 and 80 mM or between 20 and 50 mM, for example 34 mM (for example 0.2% NaCl) or 68 mM (for example 0.4% NaCl).

For example, it is considered that a stronger signal may be obtained with 0.2% NaCl, for example, than with 0% NaCl or 0.4% NaCl, though these may still be stronger than with 0.6% or 0.8% (or higher) NaCl; for example, with antibody 3D11 HRP conjugate. Similar results may be obtained with other similar antibodies, for example antibodies with specificity for the Salmonella core oligosaccharide, for example designated G7 or a similar commercially available monoclonal antibody with specificity for the core oligosaccharide of Salmonella, discussed further below; and/or with other antibodies that may be useful in Salmonella detection, for example as discussed further below.

Similar results may also be obtained with KCl, for example, a further monovalent salt.

In one embodiment, the resultant molar concentration of monovalent ions and/or salts during the exposure of the captured test sample to the detection member is less than physiological concentration of salt.

The physiological concentration of salt is considered to be about 137 mM (0.8% NaCl). In one embodiment, the resultant molar concentration of monovalent ions and/or salts during the exposure of the captured test sample to the detection member is less than 137 mM (0.8% NaCl).

In a further embodiment, the resultant molar concentration of monovalent ions and/or salts during the exposure of the captured test sample to the detection member is between 100 mM and 110 mM, for example 102.7 mM (0.6% NaCl).

In a further embodiment, the resultant molar concentration of monovalent ions and/or salts during the exposure of the captured test sample to the detection member is between 60 mM and 70 mM, for example 68.4 mM (0.4% NaCl).

In a further embodiment, the resultant molar concentration of monovalent ions and/or salts during the exposure of the captured test sample to the detection member is between 30 mM and 40 mM, for example 34.2 mM (0.2% NaCl).

In one embodiment, the monovalent ions and/or salts during the exposure of the captured test sample to the detection member are lithium, sodium and/or potassium ions and/or salts.

In an embodiment, the monovalent salt present during the exposure of the captured test sample to the detection member is LiCl, NaCl and/or KCl.

In an embodiment, the immobilised test sample and second binding member during the exposure of the captured test sample to the detection member are exposed to a resultant molar concentration of LiCl, NaCl and/or KCl of no more than 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM 125 mM 130 mM 135 mM 140 mM, 145 mM or 150 mM. Typically, they are exposed to a resultant molar concentration of LiCl, NaCl and/or KCl of between 0, 15 or 20 mM and 137, 120, 110, 100, 80, 70 or60 mM, for example 30 to 40 mM, for example 34 mM.

In one embodiment, the resultant molar concentration of LiCl, NaCl and/or KCl in during the exposure of the captured test sample to the detection member is equivalent to or less than the physiological concentration of LiCl, NaCl and/or KCl.

In one embodiment, the resultant molar concentration of LiCl, NaCl and/or KCl present in during the exposure of the captured test sample to the detection member is less than 137 mM.

In a further embodiment, the resultant molar concentration of LiCl, NaCl and/or KCl during the exposure of the captured test sample to the detection member is between 130 mM and 140 mM, for example 137 mM.

In a further embodiment, the resultant molar concentration of LiCl, NaCl and/or KCl present during the exposure of the captured test sample to the detection member is between 100 mM and 110 mM, for example 102.7 mM.

In a further embodiment, the resultant molar concentration of LiCl, NaCl and/or KCl present during the exposure of the captured test sample to the detection member is between 60 mM and 70 mM, for example 68.4 mM.

In a further embodiment, the resultant molar concentration of LiCl, NaCl and/or KCl present during the exposure of the captured test sample to the detection member is between 30 mM and 40 mM, for example 34.2 mM.

In one embodiment, the resultant molar concentration of LiCl, NaCl and/or KCl at about 137 mM, 102.7 mM, 68.4 mM or 34.2 mM or less during the exposure of the captured test sample to the detection member is suitable for detecting Salmonella in a test sample when the detection member is a mouse monoclonal antibody with specificity for the core oligosaccharide, such as antibodies designated 3D11, G7, M181, T6 or similar commercially available monoclonal antibodies with specificity for the core oligosaccharide of Salmonella; and/or when detection member(s) useful in Salmonella detection are present.

As discussed above, the inventors have surprisingly found that reduced levels of monovalent ions and/or salts, such as sodium, lithium and potassium ions and/or salts in the buffer for incubation with the detection member, significantly improves the sensitivity of the assay. To the applicant's knowledge, there is no indication in the art that teaches that a lower level of monovalent ions and/or salts present during incubation of the test sample with the at least one detection member may improve the sensitivity of the assay.

In addition, the applicant has surprisingly found that NaCl (for example) at a resultant molar concentration of 34.2 mM (0.2% w/v) during step (iv), produces a greater signal to noise ratio than higher resultant molar concentrations of NaCl, such as 68.4 mM, 102.7 mM or 137 mM, when Salmonella strains are present at lower concentrations, such as 1×10⁵ cfu/ml (see Example 3). In addition, NaCl, at lower resultant molar concentrations, improves assay sensitivity such that it is possible to detect strains of Salmonella, such as Salmonella typhimurium, at concentrations down to 1×10⁴ cfu/ml.

It is important to note that antibodies are typically assayed at physiological salt concentrations or above, and that selecting a resultant molar salt concentration that is lower than a physiological concentration is not conventional practice in the field. The applicant has shown that a conjugate buffer comprising a lower salt concentration gives rise to an unexpected and technically advantageous effect on assay sensitivity.

In an alternative embodiment, the buffer of the second binding member may comprise magnesium ions and/or salts.

Without wishing to be bound by any theory, whilst one culture method according to the disclosure allows the rapid culture and production of sufficient numbers of target Gram negative cells such as Salmonella cells or E. coli cells to allow detection by many methods, such as PCR and other molecular based methods, it has particular advantages when the detection methods involves an ELISA method, as described herein. It is considered that without a culture method according to the disclosure, standard ELISA assays are not sensitive enough to detect the target Gram negative cells such as Salmonella cells or E. coli cells after a short culture period of for example 16 to 20 hours. The sensitivity of a method of quantification or detection is significantly enhanced by employing the low salt concentrations during exposure of the sample to the detection member. Accordingly, the combination of a culture method according to the disclosure and a method of detection/quantification using a detection member at low salt concentrations is considered to have a synergistic positive effect on sensitivity.

Again, the skilled person will appreciate that following exposure to the detection member, any unbound detection member needs removal. Accordingly in one embodiment the disclosed methods further comprise exposing the detection complex to a wash solution comprising:
a) monovalent ions wherein the molar concentration of monovalent ions and/or salts 0.5 in the wash solution is at least 100, 137, 150, 200, 250, 300, 350, 400, 450, 500, 550, 600 or 650 mM, or between 100 and 700 mM or 750 mM or 800 mM, or between 300 and 650 mM, optionally wherein the molar concentration of monovalent ions and/or salts present in the wash solution is at least 200 mM 300 mM, 400 mM, 500 mM, 600 mM, 700 or 800 mM optionally 471 mM, 513.3 mM or 599 mM.
optionally wherein the monovalent ion and/or salt in the wash solution is a lithium, sodium or potassium ion and/or salt, optionally wherein the monovalent salt is LiCl, NaCl and/or KCl;
   or
b) divalent ions and/or salts wherein the molar concentration of divalent ions and/or salts in the wash solution is between 1 and 30 mM, optionally between 5 and 15 mM, between 12 and 14 mM or between 5 and 8 mM, optionally 6 to 12 mM, optionally 8.4 mM, 6.6 mM, 13 mM or 26 mM,
optionally wherein the divalent ion and/or salt in the wash solution is a magnesium or calcium ion and/or salt, optionally wherein the divalent salt is MgCl₂ and/or CaCl₂.

It will be appreciated that the detection of the detection member is an indication of the presence of the target Gram negative cells such as Salmonella cells or E. coli cells in the original test sample. Accordingly, in one embodiment of a method said detection of the presence or absence of the target Gram negative cells such as the Salmonella cells or the E. coli cells or the quantification of target Gram negative cells such as Salmonella cells or E. coli cells comprises detection of the presence or absence of the detection member, for example wherein said detection of the presence or absence of the detection member occurs after exposure to a wash solution as described above.

The skilled person will understand the various means available to allow detection of the detection member, for example said detection of the presence or absence of the detection member may comprise exposure to an antibody with specificity for the detection member, optionally wherein the antibody is an enzyme-conjugated antibody, optionally a HRP-conjugated antibody. Preferably the enzyme results in an observable colour change, optionally results in an observable colour change upon addition of the enzyme substrate.

A method may, for example, be performed in a similar manner to that described in the examples, for example in Example 5. Typically, the test sample may be heated to 90-100° C. for 15-20 minutes, and allowed to cool to room temperature prior to the assay. In a further embodiment, the inner surface of the microplate is coated with the capture member, for example, a sheep polyclonal antibody raised against the target Gram negative bacteria such as Salmonella or E. coli. In a further embodiment, the first capture binding member is bound to the assay plate via an intermediate binding member. The test sample may then be aspirated, the wells washed in wash buffer, for example comprising 599 mM (3.5%) NaCl or 8.4 mM (0.08%) MgCl₂, and then exposed to a detection member such as a HRP conjugated antibody with specificity for the core oligosaccharide of Salmonella, and incubated at 37±1° C. for 30 mins. In another embodiment, the second binding member is an antibody designated 3D11, G7, M181, T6 or a similar commercially available monoclonal antibody with specificity for the core oligosaccharide of Salmonella. In another embodiment, the captured test sample is also exposed to another second binding member such as a HRP conjugated antibody with specificity for other microbial components. The well contents may then be aspirated and the wells washed in wash buffer, for example comprising 599 mM (3.5%) NaCl or 8.4 mM (0.08%) MgCl₂. In another embodiment, the binding of the conjugated antibody is detected by a colorimetric end point detection system. As an example, substrate for the HRP, for example, may then be added, followed by incubation and addition of a stop solution (for example, 0. M H₂SO₄) as needed, for example as described in Example 4.

In an embodiment, the detection method should be capable of detecting/differentiating 1 colony forming unit (cfu) of Salmonella, in as many as 10⁸-10⁹ cfu of another microorganism such as E. coli, for example, or per swab, starting sample, and the like. Particular detection limits are about 10⁴-10⁶ cfu per unit of sample size (mg, g and the like) or volume (ml, L and the like).

As discussed above, for this and any other aspect of the disclosure, any particular preference for a particular feature from one aspect may be combined with any other particular preference for another feature in the same or a different aspect.

One embodiment provides a method for quantifying or detecting the presence or absence of a target Gram negative bacteria such as Salmonella or E. coli in a test sample wherein the method comprises:
a) culturing the microbial cells in the test sample, optionally wherein the microbial cells are cultured according to a method of the first aspect of the disclosure;
b) optionally treating the cultured microbial cells so as to cause release of bacterial LPS and/or core oligosaccharide from any microorganism present within the test sample;
c) exposing the cultured cells or the bacterial LPS and/or core oligosaccharide of the treated cultured test sample to a capture member, resulting in captured cells or captured bacterial LPS and/or core oligosaccharide, optionally wherein the capture member is a sheep polyclonal antibody, or a monoclonal antibody such as any of the 3D11, G7, M181 or T6 antibodies raised against Salmonella;
d) exposing the captured cells or captured bacterial LPS and/or core oligosaccharide to a detection member to form a detection complex wherein the detection member is an antibody with specificity for Salmonella, wherein said exposing to said detection member is performed at a molar concentration of monovalent ions and/or salts that is:
   i) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
   ii) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
   iii) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
      optionally wherein the monovalent ion and/or salt is a lithium, sodium and/or potassium ion and/or salt, optionally wherein the monovalent salt is LiCl, NaCl and/or KCL.
f) detecting the presence or absence of the detection complex, optionally wherein said detecting comprises exposure of the detection complex to an enzyme-conjugated antibody with specificity for the detection member, optionally a HRP-conjugated antibody or other suitably labelled antibody, for example a fluorophore labelled antibody;
   wherein detection of the presence of the detection complex indicates the presence of the target Gram negative cells such as Salmonella or E. coli in the initial test sample.

Capture and Detection Member

Typically, the one or more capture and detection member(s) used in various ways in the assay is an antibody, more particularly an affinity-purified antibody and yet more particularly a polyclonal or monoclonal antibody. An antibody for use in an assay according to the present disclosure may be a polyclonal, monoclonal, bispecific, humanised or chimeric antibody. Such antibodies may consist of a single chain but would consist of at least a light chain or a heavy chain, but it will be appreciated that at least one complementarity determining region (CDR) is required in order to bind a target such as a core oligosaccharide or microbial contaminant to which the antibody has binding specificity.

In one embodiment, the capture or detection member has binding specificity to a core oligosaccharide or microbial fragment of the target Gram negative bacteria such as Salmonella or E. coli. Thus, for example, the one or more capture members and/or the one or more detection members may have binding specificity to a core oligosaccharide or microbial fragment of Salmonella or E. coli. Typically, the one or more capture members may differ from the one or more detection members. For example, the capture member(s) and the detection binding member(s) may be prepared in different ways and/or be considered to bind to different epitopes (even if there may be some overlap between the epitopes recognised by the capture member(s) and the detection member(s).

In one embodiment, the at least one capture member has binding specificity for somatic and flagella based Salmonella antigens.

In one embodiment, the at least one capture member is a sheep polyclonal antibody. In one embodiment, the purified capture member is biotinylated.

In another embodiment, the detection member has binding specificity for an antigen or antigens that are present on the Gram negative bacteria such as Salmonella or E. coli that may not have the conserved LPS/core oligosaccharide epitope(s) that are present in the vast majority of other Gram negative bacteria such as Salmonella or E. coli. Such antigens may not be unique to the target Gram negative bacteria such as Salmonella or E. coli, but may be useful, particularly when a first capture antibody is used that may be specific for the target Gram negative bacteria such as Salmonella or E. coli, in detecting Salmonella or E. coli that may not have the conserved Salmonella or E. coli LPS/core epitope. For example, one or more of the detection members, for example, may have binding specificity for certain forms of the O antigen or specificity for other subspecies of Salmonella or E. coli such as Salmonella enterica subsp. arizonae. Such a detection member may be used alongside a further one or more second detection members (for example) that have binding specificity for other Salmonella or E. coli that may not have the certain forms of the O antigen. The antibody may, for example, be a polyclonal antibody with affinity for certain weakly reacting Salmonella O epitopes. In another embodiment, the one or more detection members may be specific to the flagella of Salmonella.

In one embodiment, a detection member is a rabbit polyclonal antibody with specificity for Salmonella with certain forms of the O antigen. In another embodiment, the detection member may be a rabbit polyclonal antibody, which may react with flagella based antigens on Salmonella.

In certain embodiments, and in an optional step of a disclosed method, the complex is exposed to a secondary binding member which has binding specificity to at least one detection member (for a sufficient time to allow for the secondary binding member to form a secondary complex, for example a core oligosaccharide/binding member (for example second binding member)/secondary binding member complex.

Methods of making antibodies are known in the art. For example, if polyclonal antibodies are desired, then a selected mammal, such as a mouse, rabbit, goat, sheep or horse may be immunised with the antigen of choice, such as bacterial endotoxin. The serum from the immunised animal is then collected and treated to obtain the antibody, for instance by immunoaffinity chromatography. For example, the antibody may be affinity purified using a CNBr Sepharose column bound to insoluble Salmonella antigens.

Monoclonal antibodies may be produced by methods known in the art, and may generally be preferred, at least for some components. The general methodology for making monoclonal antibodies using hybridoma technology is well known (see, for example, Kohler, G. and Milstein, C, Nature 256: 495-497 (1975); Kozbor et al, Immunology Today 4: 72 (1983); Cole et al, 77-96 in Monoclonal Antibodies and Cancer Therapy, Alan R. Liss, Inc. (1985).

An antibody, as referred to herein, should consist of an epitope-binding region, such as CDR. The antibody may be of any suitable class, including IgE, IgM, IgD, IgA and, in particular, IgG. The various subclasses of these antibodies are also envisaged. As used herein, the term “antibody binding fragments” refers in particular to fragments of an antibody or polypeptides derived from an antibody which retain the binding specificity of the antibody. Such fragments include, but are not limited to antibody fragments, such as Fab, Fab′, F(ab′)2 and Fv, all of which are capable of binding to an epitope.

The term “antibody” also extends to any of the various natural and artificial antibodies and antibody-derived proteins which are available, and their derivatives, e.g. including without limitation polyclonal antibodies, monoclonal antibodies, chimeric antibodies, humanized antibodies, human antibodies, single-domain antibodies, whole antibodies, antibody fragments such as F(ab′)2 and F(ab) fragments, Fv fragments (non-covalent heterodimers), single-chain antibodies such as single chain Fv molecules (scFv), minibodies, oligobodies, dimeric or trimeric antibody fragments or constructs, aptamers and affimers etc. The term “antibody” does not imply any particular origin, and includes antibodies obtained through non-conventional processes, such as phage display. Antibodies of the disclosure can be of any isotype (e.g. IgA, IgG, IgM i.e. an a, y or p. heavy chain) and may have a K (kappa) or an A (lambda) light chain.

Accordingly, reference to antibody or antibody fragment throughout is intended to encompass the meaning of the various types of antibody or antibody fragment, for example aptamers or affimers.

The present disclosure therefore extends to the use of antibodies and antibody derived binding fragments which have binding specificity to core oligosaccharides for use in the present invention.

The term “specifically binds” or “binding specificity” refers to the ability of an antibody or fragment thereof to bind to a target microbial pathogen with a greater affinity than it binds to a non-target epitope. For example, the binding of an antibody to a target epitope may result in a binding affinity which is at least 10, 50, 100, 250, 500, or 1000 times greater than the binding affinity for a non-target epitope. In certain embodiments, binding affinity is determined by an affinity ELISA assay. In alternative embodiments, affinity is determined by a BIAcore assay.

Alternatively, binding affinity may be determined by a kinetic method.

In certain embodiments, the capture member (capture member or members), such as an antibody, may be immobilised on the surface and after an optional washing step, the test sample, which may contain the core oligosaccharide or microbial contaminant of interest can be exposed to the surface-bound antibody for a sufficient time for binding to take place and a surface bound first (capture) binding member-core complex to form. The assay may then involve a step of exposing the surface bound first capture member-core complex to one or more detection members, such as an antibody or antibodies, which may be covalently conjugated with means for light emission, for example, an acridinium ester. In such cases, the detection member has binding specificity for an epitope present on the core oligosaccharide or microbial contaminant, so that the amount of signal generated corresponds to the amount of core oligosaccharide or microbial contaminant bound by the primary and second binding member(s).

Typically, an antibody may be purified to reduce aggregation.

In certain embodiments the surface is, for example, a microtitre plate of conventional design, but an advantage may be gained by using a modified surface, for instance having darkened side walls and a white or transparent portion (e.g. on the base), for example if a chemiluminescent assay is being used. This can intensify any signal generated and reduces the background light at the time of measurement. The white portion allows reflection of the light to intensify the generated signal. Thus, in particular embodiments, the surface is a multi-well plate comprising a plurality of wells, wherein the base of each well is transparent or substantially transparent, while the walls of the wells are opaque, or darkened to prevent the passage of light, or coloured to provide a contrast against the base portion of the well which allows light to pass there through.

Yet more particularly the antibody (for example first capture antibody; or second detection antibody) is a species specific antibody, for example a species specific monoclonal antibody. Use of the term ‘species specific’ is intended to mean that such an antibody will differentiate between, for example, Salmonella, Shigella and other Gram-negative species, for example, with little or no cross-reaction. However, it may not be necessary for one or more detection members, for example antibody, for example, to be species specific if the first (capture) antibody or antibodies is species specific. Thus, for example a detection member/antibody that binds to a form of the O antigen (which is not species specific) may be useful, for example when the first (capture) binding member is considered to be species specific for Salmonella and/or alongside another second (detector) binding member that may be able to bind to most but not all Salmonella serovars: the O antigen-directed second (detector) binding member may assist in binding to serovars that may not be bound by the main second (detector) binding member.

In particular embodiments, the capture or detection members (for example one or more second (detector) binding member) may interact with and bind to the:

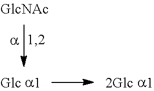

epitope of the LPS core oligosaccharide. This epitope is generally species specific differentiating Salmonella from most other bacteria such as, by way of non-limiting example, Shigella, Listeria, E. coli etc. In particular embodiments the assay method is a method for the quantitative detection of a target Gram negative bacteria, such as Salmonella or E. coli. An assay method may also be utilised to detect for the presence or absence of the target Gram negative bacteria, such as Salmonella or E. coli. In particular embodiments the detection member is a labelled binding member labelled by, for example, conjugation to an enzyme label such as horse radish peroxidase, chemiluminescent or fluorescent compound.

Examples of such second binding members include mouse monoclonal antibodies designated Solus antibody 3D11 or similar antibodies which are commercially available, with specificity for the core lipopolysaccharide antigen on Salmonella.

Other examples include antibodies as mentioned in http://www.virostat-inc.com/content/page/catalog/keyword/Salmonella/search.html?keyword=Salmonella.

A further example of a suitable antibody may be M181 (Brooks et al Canadian J Vet Research (2014) 78, 68-71; Brown et al (1996) Immunol Invest 25, 369-381.) and T6 monoclonal antibody (Ng 1996 Appl Environ Microbiol. 1996 July; 62(7):2294-302).

In a further embodiment, the capture binding member is a polyclonal Ab which may be specific for the “O” side chains of the LPS as well as many other cell components.

Different binding members, for example antibodies, may have different, useful or optimal arrangements, for example whether to immobilise passively or using streptavidin, for example, as can be determined by those skilled in the art.

Detection

The disclosed methods further comprise detecting any binding of the at least one detection member to a core oligosaccharide or microbial contaminant of the microorganism of interest, although the disclosed methods shall not be limited to a particular detection methodology.

For example, the detection methods may be by any suitable method known in the art such as by colourimetry, fluorescence measurement, flow cytometry, chemiluminescence and the like. In one embodiment, detection of binding is by measurement/detection of a luminescent signal, for example, chemiluminescent light produced by a chemiluminescent compound. Suitable chemiluminescent compounds include acridinium esters, acridinium sulfonamides, phenanthridiniums, 1,2-dioxetanes, luminol or enzymes that catalyse chemiluminescent substrates and the like.

In an embodiment, a colourimetric end point detection system is used for the detection of the target Gram negative bacteria, such as Salmonella or E. coli, particularly in a food testing environment.

In certain embodiments the detection member may be conjugated directly to detectable moiety.

Detectable moieties are well known in the art.

In one embodiment, the substrate can be purchased as a proprietary solution from Interchim.http://www.interchim.com/catalogue.php?ref=406&varnombre=25&var0=1&limite=50

In another embodiment, the enzymes that catalyse the substrate can be stopped after a period of time, for example, with 0.1M H₂SO₄.

In one embodiment, the detectable moiety is a light-emitting moiety. In certain embodiments the detection member is conjugated to an acridinium compound or derivative thereof, such as an acridinium ester molecule or acridinium sulphonamide which acts as a luminescent label. In embodiments where the antibody or binding fragment is conjoined to an acridinium ester or acridinium sulphonamide, the assay method may further comprise the step of adding AMPPD to the test sample.

AMPPD may also be known by the synonyms: 3-(2′-spiroadamantane)-4-methoxy-4-(3″-phosphoryloxy)phenyl-1,2-dioxetane; 3-(4-methoxyspiro(1,2-dioxetane-3,2-tricyclo(3.3.1.1(3,7))decan)-4-yl)phenyl phosphate; 4-methoxy-4-(3-phosphatephenyl)spiro(1,2-dioxetane)-3,2′-adamantane.

In certain further embodiments, an antibody (or other detection member) may be indirectly associated with a light-emitting moiety, for example the acridinium ester molecule may be conjugated to a second antibody which is capable of binding to the first antibody.

In certain embodiments, one or more luminescent or fluorescent moieties may be bound to avidin/streptavidin, which in turn may be bound to biotin chemically conjugated to an antibody. In certain further embodiments, lectins (Protein A/G/L) can be linked to a luminescent or fluorescent molecule which may also be attached to an antibody or other protein conjugate.

In one embodiment, the moiety is Horse Radish Peroxidase.

The stimulus to produce a detectable signal can be light, for example, of a particular wavelength, e.g. UV light, or may be some other stimulus such as an electrical or radioactive stimulus, a chemical or enzyme-substrate reaction.

In other embodiments the detection member, e.g., an antibody with binding specificity to a core oligosaccharide or microbial fragment of the target Gram negative bacteria, such as Salmonella or E. coli, may be indirectly associated with such a light-emitting moiety, for example, the acridinium ester molecule may be conjugated to a further binding member which is capable of binding to the second binding member.

In certain embodiments, the assay methods may be qualitative or quantitative, and standard controls can be run to relate the average signal generated to a given quantity of, for example, core oligosaccharide.

In alternative embodiments, the assay methods may suitable for detecting the presence or absence of a target Gram negative bacteria, such as Salmonella or E. coli.

In certain embodiments, the disclosed methods may be used for the determination in a sample of a plurality of core oligosaccharides or microbial contaminants, this being achieved by providing a plurality of binding members such as antibodies each of which having binding specificity to a different epitope or microbial contaminant.

It should be apparent that between or at each stage of a method, optional washing, drying and/or incubation steps may be included. The methods may also optionally include ‘blocking steps’ between one or more steps of a method wherein a concentrated solution of a non-interacting protein, such as bovine serum albumin (BSA) or casein, is added, for example to all wells of a microtitre plate. Particular blocking agents also include solutions of milk powder and the like. Such proteins block non-specific adsorption of other proteins to the plate and may be beneficial in reducing ‘background’ artefacts which can interfere with the sensitivity of the assay.

A wash step may be a “high salt” wash step, for example comprising aspirating the test sample and then washing the wells/complex with a wash solution comprising a high concentration of salt as discussed above as an aspect of the disclosure.

In another embodiment, the wash step may comprise aspirating the test sample and then washing the wells/complex with a wash solution comprising MgCl₂ as discussed above as an aspect of the disclosure.

The disclosure typically makes use of a binding member which has binding specificity to a core oligosaccharide for the specific detection of the target Gram negative bacteria, such as Salmonella or E. coli.

It will be clear to the skilled person that the various components required to carry out the methods, compositions and uses described herein may be provided in the form of a kit or a kit of parts. For example, a further aspect of the disclosure provides a kit for the selective culture of Gram negative cells such as Salmonella cells or E. coli cells, optionally Gram negative bacteria such as Salmonella cells or E. coli cells that may be present in a test sample, optionally wherein the culture is performed according to a method of the first aspect of the disclosure or for carrying out any of the methods described herein wherein the kit comprises:

an agent of Formula I as defined in the first aspect of the disclosure; and

Cefsulodin and/or novobiocin;

optionally wherein the agent of Formula I is 4PYcq.

The disclosure also provides a kit for use in a method of quantifying or detecting the presence or absence of target Gram negative cells such as Salmonella cells or E. coli cells, optionally for detecting the presence or absence of target Gram negative cells such as Salmonella cells or E. coli cells in a test sample, optionally wherein a method of quantifying or detecting the presence or absence of the target Gram negative cells such as Salmonella or E. coli comprises a method of selective culture according to the first aspect of the disclosure, wherein the kit comprises any two or more of:
a) an agent of Formula I as defined in the first aspect of the disclosure, optionally wherein the agent of Formula I is 4PYcq;
b) Cefsulodin and/or novobiocin;
c) an agent to treat microbial cells so as to cause release of bacterial LPS and/or core oligosaccharide from any microorganism present, optionally alcohol or a water based buffer;
d) a capture member,
   optionally wherein the capture member is an antibody or antibody fragment, optionally a biotinylated antibody or antibody fragment,
   optionally wherein the capture member is specific for microorganisms, optionally specific for bacteria, optionally specific for Gram negative bacteria, optionally specific for Salmonella or E. coli, optionally wherein the capture member is a sheep polyclonal antibody raised against Salmonella
      optionally wherein the capture member is immobilised to a substrate or solid surface, optionally immobilised to a substrate or solid surface via an intermediate binding partner, optionally wherein the intermediate binding partner is a linkage comprising biotin and streptavidin;
e) a detection member, optionally
   wherein the detection member is an antibody or antibody fragment; and/or
   wherein the capture member is specific for microorganisms, optionally specific for bacteria, optionally specific for Gram negative bacteria, optionally specific for Salmonella or E. coli; and/or
   wherein the detection member binds to the captured cells or captured bacterial LPS and/or core oligosaccharide to form a detection complex; and/or
   wherein the detection member is an antibody selected from the group consisting of
   3D11, G7, M181 and T6
   or ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS;
      Salmonella typhi 6301, 6321, 6345, 6331, 6347;
f) a wash solution comprising monovalent ions wherein the molar concentration of monovalent ions and/or salts in the wash solution is at least 100, 137, 150, 200, 250, 300, 350, 400, 450, 500, 550, 600 or 650 mM, or between 100 and 700 mM or 750 mM or 800 mM, or between 300 and 650 mM
g) a wash solution comprising divalent ions and/or salts wherein the molar concentration of divalent ions and/or salts in the wash solution is between 1 and 30 mM, optionally between 5 and 15 mM:
h) a culture medium as defined in the preceding aspects of the disclosure;
i) an antibody with specificity for the detection member, optionally wherein the antibody is an enzyme-conjugated antibody, optionally a HRP-conjugated antibody.
j) streptavidin assay plates, wherein the inner surface of the wells is coated in biotinylated polyclonal antibodies with specificity for the target Gram negative bacteria such as Salmonella or E. coli or microbial fragments thereof;
k) instructions for performing a method of the first, second and third aspects of the disclosure;
l) ferric chloride; and/or
m) a sulphite compound such as potassium sulphite.

In one embodiment the kit comprises:

an agent of Formula I as defined in the first aspect of the disclosure (a); and any one or more of Cefsulodin (b), novobiocin (b), ferric chloride (1) or a sulphite compound such as potassium sulphite (m),

optionally wherein the agent of Formula I is 4PYcq.

In one embodiment the kit comprises an agent of Formula I as defined in the first aspect of the disclosure (a), and Cefsulodin (b), and novobiocin (b); and optionally ferric chloride (1) and/or a sulphite compound such as potassium sulphite (m), optionally wherein the agent of Formula I is 4PYcq.

In another embodiment, the kit comprises an agent of Formula I as defined in the first aspect of the disclosure (a), and Cefsulodin (b), and novobiocin (c) and ferric chloride (1) and a sulphite compound such as potassium sulphite (in),

optionally wherein the agent of Formula I is 4PYcq.

In yet a further embodiment, the kit comprises a composition comprising an agent of Formula I as defined in the first aspect of the disclosure (a), Cefsulodin (b) and Novobiocin (c); and further comprises a second composition that comprises ferric chloride and a sulphite compound such as potassium sulphite, as described above in relation to earlier aspect of the disclosure. In some embodiments, the second composition is a culture media, or is a lyophilised culture media that comprises the ferric chloride and the sulphite compound such as potassium sulphite.

Where the kit is for use in a method of quantifying or detecting the presence or absence of a target Gram negative bacteria such as Salmonella or E. coli in a sample that is a spice, herb or flavouring, it is considered to be advantageous if the kit comprises ferric chloride (1) and a sulphite compound such as potassium sulphite (m); or comprises a composition comprising ferric chloride and a sulphite compound such as potassium sulphite.

In one embodiment the kit does not comprise ferric chloride and/or a sulphite compound such as potassium sulphite.

Although it is expected that the main application of the present disclosure will be in industrial food and environmental testing, it will be apparent that the improved method of selectively culturing Gram negative bacteria such as Salmonella cells or E. coli cells, either alone or in conjunction with a method of detecting or quantifying target Gram negative cells such as Salmonella cells or E. coli cells as described herein has medical applications, for example in improved and faster detection of Salmonella infections for example. Accordingly, the disclosure also provides a method of diagnosing a subject as being infected with the target Gram negative bacteria such as Salmonella or E. coli wherein a method comprises culturing the microbial cells in a sample according to the first aspect of the disclosure, or detecting the presence or absence of the target Gram negative bacteria such as Salmonella or E. coli according to later aspects of the disclosure.

The disclosure also provides a method for treating a subject that has been diagnosed as infected with the target Gram negative bacteria such as Salmonella or E. coli wherein the diagnosis is performed according to or involves any of the methods or compositions as described herein.

The disclosure also provides an anti-Gram negative therapy such as an anti-Salmonella therapy or an anti-E. coli therapy for use in treating a subject that has been diagnosed as infected with the target Gram negative bacteria, such as Salmonella or E. coli wherein the diagnosis is performed according to or involves any of the methods or compositions as described herein.

In a further aspect, and as discussed above, the inventors have surprisingly found that reduced levels of monovalent ions and/or salts, such as sodium, lithium and potassium ions and/or salts in the buffer for incubation with the detection member, improves the sensitivity of the assay. Accordingly, the disclosure also provides a method for the detection of the presence of a target Gram negative cell such as Salmonella cells or E. coli cells in a sample comprising or expected to comprise microbial cells, optionally wherein the sample is a sample of cultured microbial cells that is independent of the means by which the target Gram negative cells such as Salmonella cells or E. coli cells are cultured.

The disclosure provides a method for the detection of the presence of target Gram negative cells such as Salmonella cells or E. coli cells in a sample comprising or expected to comprise microbial cells, optionally wherein the sample is a sample of cultured microbial cells,
wherein the method comprises an immunoassay wherein the immunoassay comprises the use of a detection member, wherein the sample or microbial cells are exposed to the detection member at a salt concentration of:
   a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
   b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
   c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
   optionally wherein the monovalent ion and/or salt is a lithium, sodium and/or potassium ion and/or salt, optionally wherein the monovalent salt is LiCl, NaCl and/or KCl.

The disclosure also provides a method for the detection of the presence of a target Gram negative cell such as Salmonella cells or E. coli cells in a sample comprising or expected to comprise microbial cells, wherein the method comprises an immunoassay wherein the immunoassay comprises:
a) optionally treating the sample or cultured microbial cells so that the microbial cells are treated so as to cause release of bacterial LPS and/or core oligosaccharide from any microorganism present within the test sample;
b) exposing the sample or the cultured microbial cells or the bacterial LPS and/or core oligosaccharide of the treated cultured microbial cells to a capture member, resulting in captured cells or captured bacterial LPS and/or core oligosaccharide,
   optionally wherein said exposing occurs after the cultured microbial cells are treated so as to cause release of bacterial LPS and/or core oligosaccharide from any microorganism present within the test sample, optionally
wherein the capture member is an antibody or antibody fragment, optionally a biotinylated antibody or antibody fragment, optionally wherein the capture member has specificity for Salmonella, optionally wherein the capture member is a sheep polyclonal 1.5 antibody, raised against Salmonella,
optionally wherein the capture member is
   a monoclonal antibody with specificity for the LPS or core oligosaccharide of Salmonella, optionally 3D11, G7, M181 or T6 antibody;
ViroStat product number:
   Salmonella sp. 6371-6394, 6301-6347, 6301-flagella, 6321-flagella, 6331-typhimurium, 6392-common core, 6393, 6394, 6391, 6381, 6371;
   Salmonella paratyphi A 6347-LPS; or
   Salmonella typhi 6301, 6321, 6345, 6331, 6347; and
c) exposing the captured cells or captured bacterial LPS and/or core oligosaccharide to a detection member, wherein the detection member binds to the captured cells or captured bacterial LPS and/or core oligosaccharide to form a detection complex, optionally wherein the detection member is an antibody or antibody fragment, optionally wherein the detection member has specificity for Salmonella, optionally wherein the detection member is selected from the group consisting of:
   a monoclonal antibody with specificity for the LPS or core oligosaccharide of Salmonella, optionally 3D11, G7, M181, or T6 antibody,
   ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS; or
      Salmonella typhi 6301, 6321, 6345, 6331, 6347;
         and
   wherein the captured cells or captured bacterial LPS and/or core oligosaccharide are exposed to the detection member at a salt concentration of:
      a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
      b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
      c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
      optionally wherein the monovalent ion and/or salt is a lithium, sodium and/or potassium ion and/or salt, optionally wherein the monovalent salt is LiCl, NaCl and/or KCl.

Preferences for these aspects of the disclosure are as discussed above for previous aspects of the disclosure.

As discussed above, any particular preference for a particular feature may be combined with any other particular preference for another feature.

The disclosure also provides the following numbered aspects and embodiments of the disclosure:
1) A method for selectively culturing Gram negative bacteria such as Salmonella or E. coli, optionally for culturing Gram negative bacteria such as Salmonella or E. coli that may be present in a test sample, the method comprising the step of culturing microorganisms present in the test sample in a culture medium, wherein the culture medium comprises an agent of Formula I

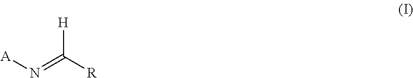

wherein A is selected from

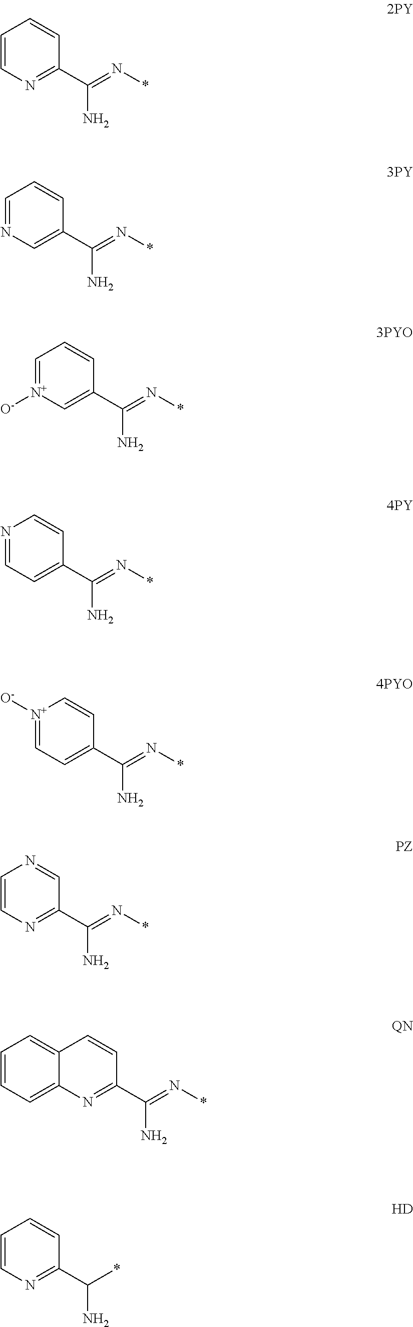

and R is selected from optionally substituted C₅₋₂₀ aryl, with the proviso that when A is 2PY, then R is not 1,3-dimethylphenyl.
2) The method of embodiment 1 wherein where A is 2PY, 3PY, 4PY PZ QN or HD, R is selected from the group consisting of

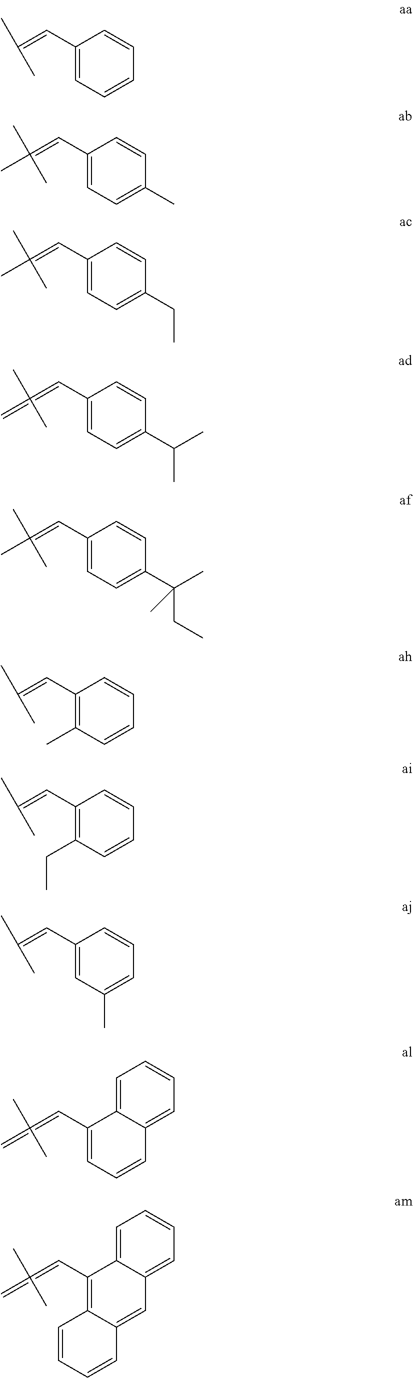

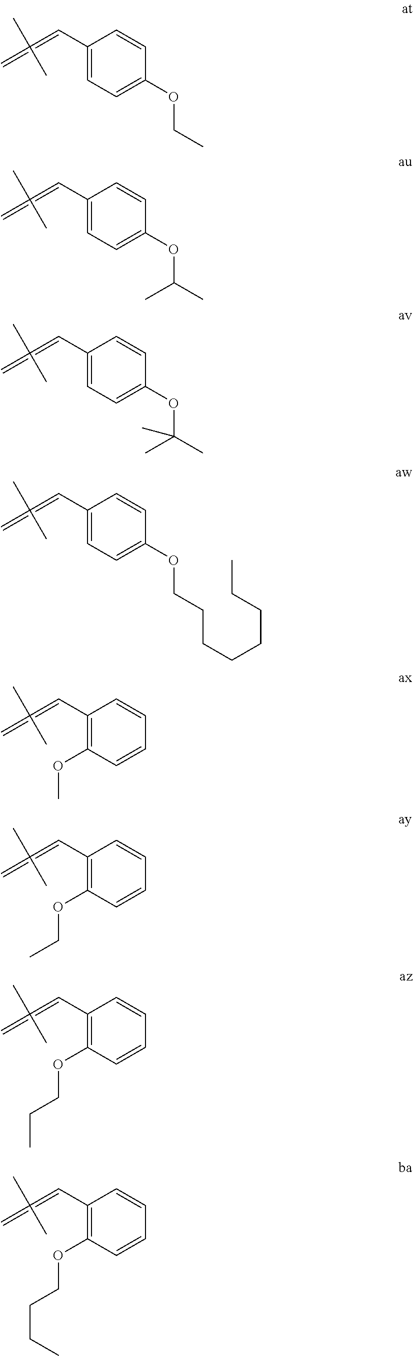

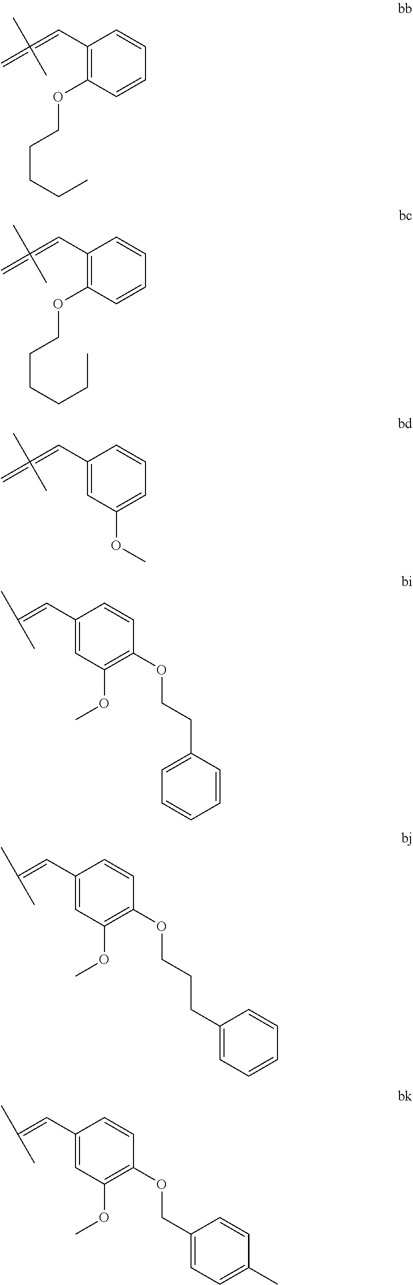

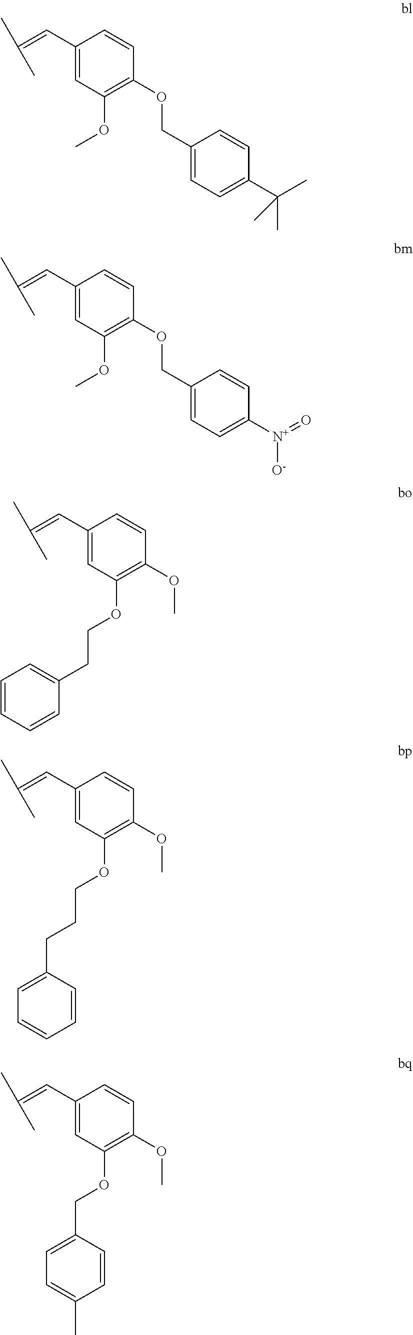

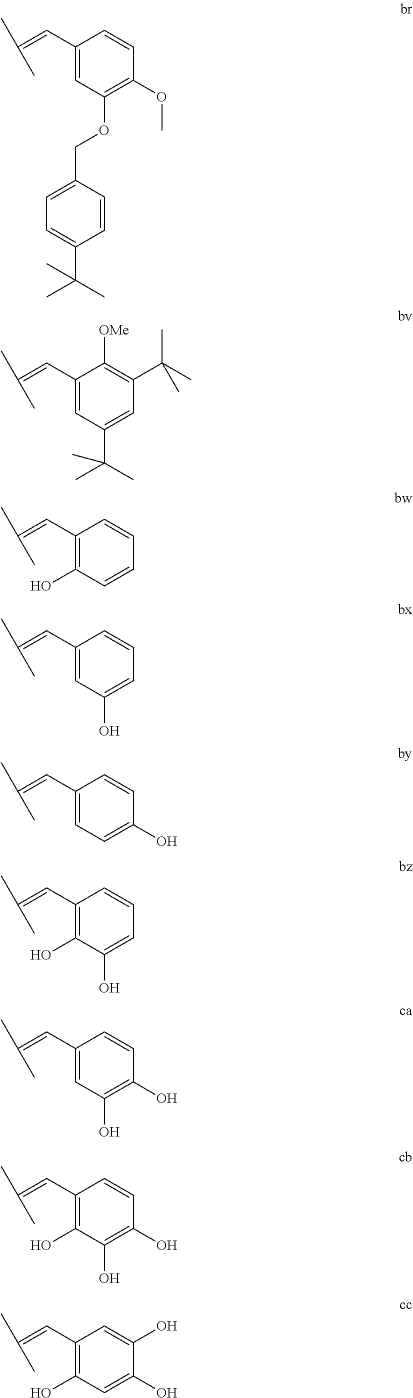

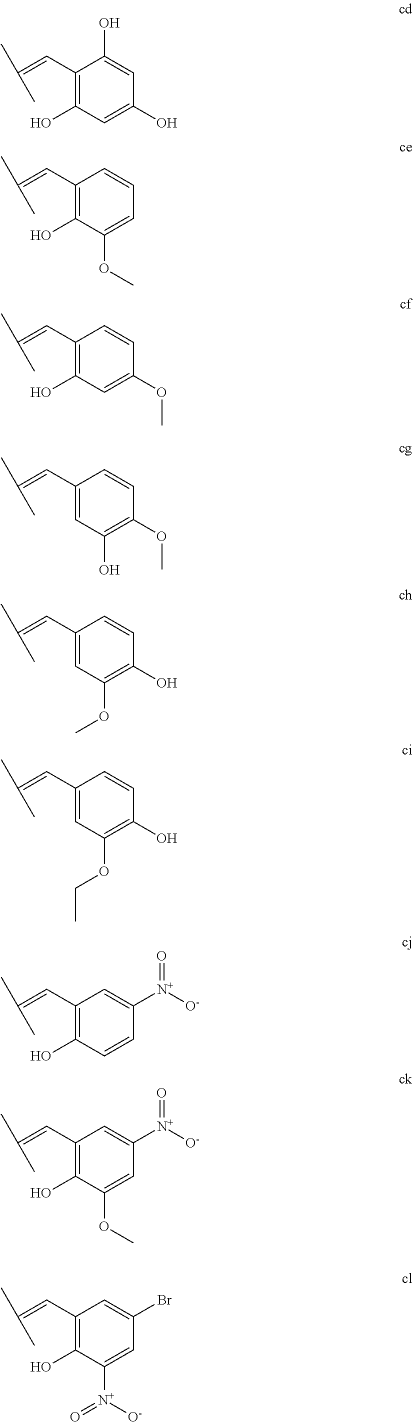

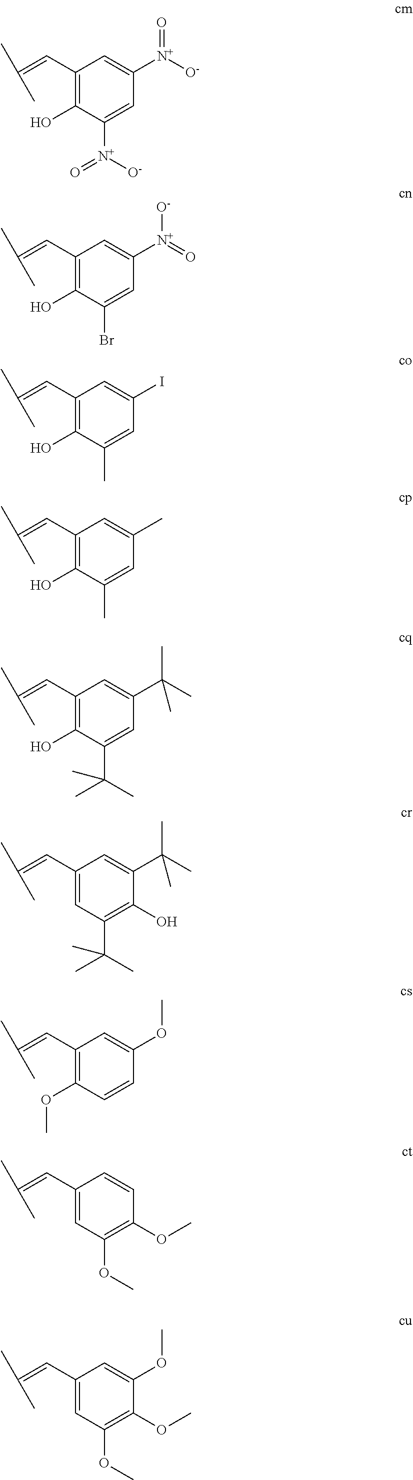

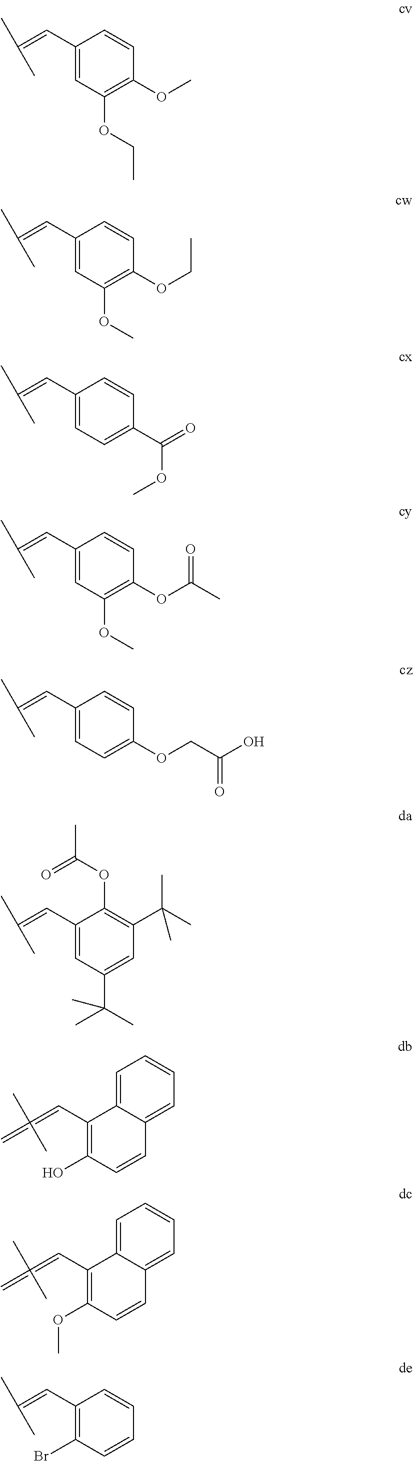

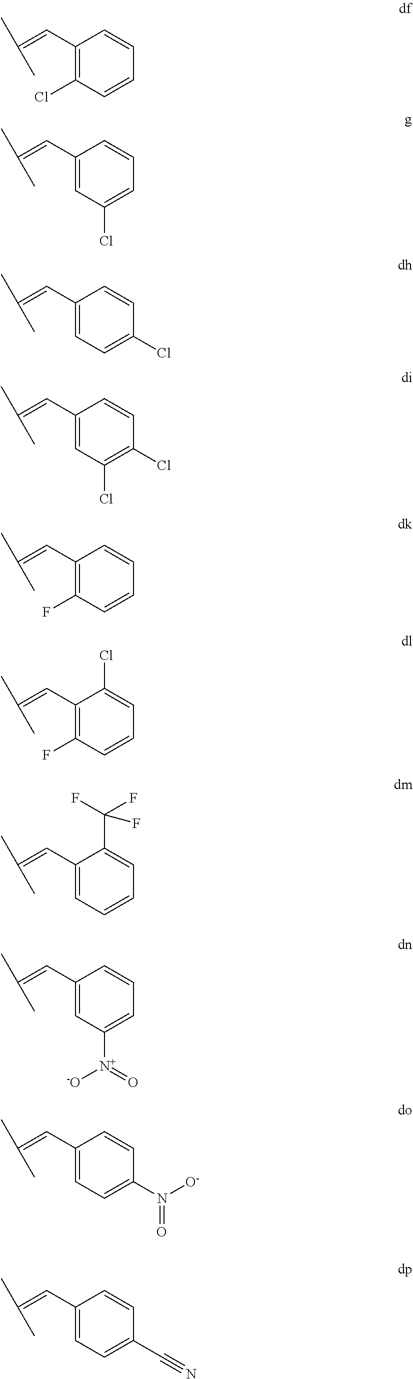

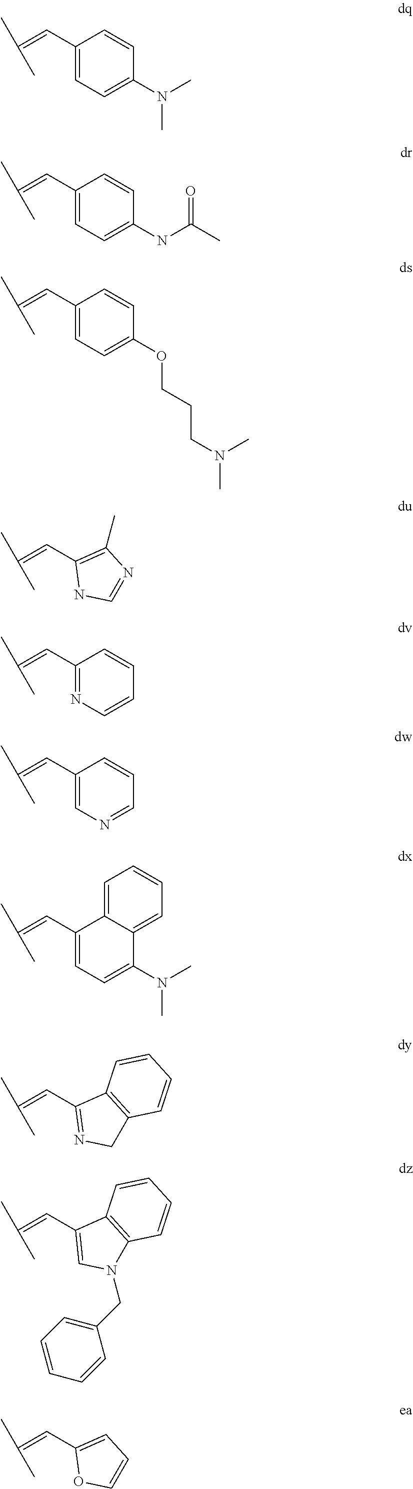

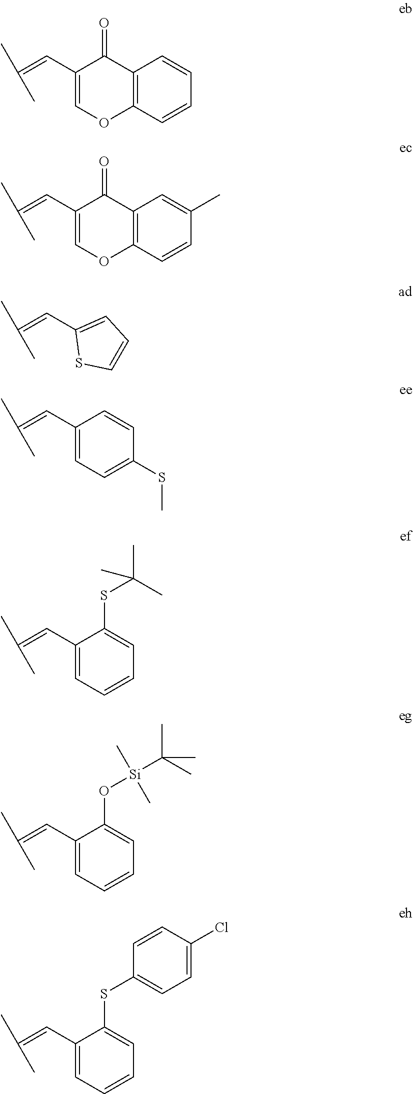

3) The method of any one of embodiments 1 or 2 wherein:
   where A is 2PY R is selected from af, ah, ai, aj, al or cj;
   where A is 3PY R is selected from af, ay, cc, cj or cl;
   where A is 4PY R is selected from af, am, cb, cc, cj co or cq;
   where A is HD R is selected from cd, ce, cf, cj or cl;
   where A is PZ R is selected from cb or cj;
   where A is QN R is ca.
4) The method of any one of embodiments 1-3 wherein the agent of Formula I is selected from the group consisting of 3PYaf, 4PYaf, 4PYam, 4PYcb, 4PYco, 4PYcq, 4PYeh, HDcb, HDce, HDcf and HDdb.
5) The method of any one of embodiments 1-4 wherein the agent of Formula I is 4PYcq.
6) The method of any one of embodiments 1-5 wherein the agent of Formula I is present in the culture media in a concentration of:
a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L; and/or
b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L; optionally 5 mg/L.
7) The method of any of embodiments 1-6 wherein the agent of Formula I is present in the culture media in a concentration of around 5 mg/L, optionally 5 mg/L.
8) The method according to any one of embodiments 1-7 wherein the culture medium further comprises one, two, three or four or more anti-bacterial agents, optionally comprises Cefsulodin and/or Novobiocin.
8a) The method according to any one of embodiments 1-8 wherein the culture medium further comprises ferric chloride and/or a sulphite compound such as potassium sulphite.
9) The method according to any of embodiments 1-8 wherein the culture medium further comprises an agent that inhibits the growth of, or kills, Pseudomonas aeruginosa and/or other pseudomonads optionally comprises Cefsulodin.
10) The method according to any of embodiments 1-9 wherein the culture medium further comprises Cefsulodin at a concentration of:
   between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L; or
   8 mg/L.
11) The method of any one of embodiments 1-10, wherein the culture medium comprises novobiocin in an amount of:
   between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   10 mg/L.
11a) The method of any one of embodiments 1-11, wherein the culture medium comprises ferric chloride in an amount of:
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/L
11b) The method of any one of embodiments 1-11a, wherein the culture medium comprises a sulphite compound such as potassium sulphite in an amount of:
   a) between around 0.5 g/L and 4 g/L, for example between 0.75 g/L and 3.5 g/L: 1.0 g/L and 3.0 g/L; 1.25 g/L and 2.75 g/L; 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
   c) at least 0.5 g/L, 0.75 g/L, 1.00 g/L, 1.25 g/L, 1.50 g/L, 1.75 g/L, 2.00 g/L, 2.25 g/L, 2.50 g/L, 275 g/L, 3.0 g/L, 3.5 g/L, or 4.0 g/L; or
   d) 1.00 g/L; or
   e) 1.11 g/L.
12) The method according to any one of embodiments 1-11 wherein the test sample is a sample that comprises or is expected to comprise at least one or more cells other than a Gram negative bacterial cell such as a Salmonella cell or E. coli cell, for example one or more other microorganism cells
   optionally wherein the test sample is expected to comprise at least one or more cells that is:
   a bacteria cell that is a Gram positive bacterial cell, and/or
   a fungal cell optionally a yeast cell.
13) The method according to any one of embodiments 1-12 wherein the test sample comprises, or is expected to comprise a heterogeneous microflora environment.
14) The method of any of embodiments 1-13 wherein the test sample is:
   a food product, optionally meat, meat products including mince, eggs, cheese, milk, vegetables, chocolate, confectionery, peanut butter and the like including processed, dried, frozen or chilled food products, a spice, a herb or a flavouring product;
   a clinical sample such as a biopsy sample, faecal, saliva, hydration fluid, nutrient fluid, blood, blood product, tissue extract;
   a therapeutic product such as a vaccine, anaesthetic, pharmacologically active agent, imaging agent or urine sample, probiotics and the like;
   a swab, for example a skin swab, a caecum swab, a faecal swab, a cloaca swab or a rectal swab; a swab of surfaces such as floors, doors and walls; a swab of food products including animal carcass swabs;
   a cosmetic sample such as foundation makeup, lip-balms, lotions, creams, shampoos and the like;
   a sample of a liquid in which a sample such as food or a swab has been suspended; or
   a sample of raw meat or raw poultry, or a swab taken from raw meat or raw poultry.
15) The method according to any one of embodiments 1-14 wherein the test sample is not a pure culture of Gram negative bacteria or not a pure culture of any one particular Gram negative bacterial species, optionally not a pure culture of Salmonella or E. coli.
16) The method according to any one of embodiments 1-15 wherein the culture is a liquid culture.
17) The method according to any one of embodiments 1-16 wherein the method allows the growth of stressed Gram negative cells such as Salmonella cells or E. coli cells, optionally has no or substantially no effect on the growth of stressed Gram negative cells such as Salmonella cells or E. coli cells relative to the level of growth of the stressed Gram negative cells such as Salmonella cells or E. coli cells in non-selective media.
18) The method according to any one of embodiments 1-17 wherein the method results in a cell density of at least 10⁴-10⁶ cfu ((target cells, such as gram negative bacteria in general, or for example Salmonella or E. coli)/ml, optionally results in a cell density of at least 10⁴-10⁶ cfu (target cells, such as gram negative bacteria in general, or for example Salmonella or E. coli)/ml following 16 hours of culture or more, optionally 20 hours of culture.
19) The method according to any one of embodiments 1-18 wherein the method comprises a single culture step, optionally at a single temperature.
20) The method of any one of embodiments 1-19, wherein the test sample is cultured in the culture medium at a temperature from 30° C. to 44° C., optionally from 37° C. to 42° C., optionally at 41.5° C.
21) The method of any one of embodiments 1-20, wherein the test sample is cultured in a culture medium for between around 10-28 hours, for example between 12-26 hours, for example between 14-24 hours, for example between 16-22 hours, for example 18-20 hours, for example 16 hours or 20 hours.
22) The method of any one of embodiments 1-21, wherein the test sample is cultured in the culture medium for less than 24 hours, optionally less than 22 hours, optionally less than 20 hours, optionally less than 18 hours, optionally 16 hours or less.
23) The method of any one of embodiments 1-22 wherein the test sample is cultured in the culture medium for 16 hours at 41.5° C., or for 2 hours at 41.5° C., optionally wherein
   where the sample is an environmental sample the sample is cultured for 16 hours at 41.5° C.;
   where the sample is a food sample the sample is cultured for 20 hours at 41.5° C.
24) The method of any of embodiments 1-23 wherein the test sample is cultured in a nutrient culture medium comprising a base broth selected from the group consisting of peptone, tryptone, nutrient broth, L-broth, Gram negative broth, tryptic soy broth with yeast, modified tryptic soy broth and buffered peptone water.
25) The method of any one of embodiments 1-24, wherein the culture medium comprises buffered peptone water.
26) The method of any one of embodiments 1-25 wherein the culture medium does not comprise any one of, any two or, or all of:
   a) vancomycin;
   b) Brilliant green; and
   c) Malachite green.
27) The method of any one of embodiments 1-26 wherein the culture medium does not comprise any one of, any two or more of, or all of:
   a) bile salts, optionally deoxycholate;
   b) Sodium thiosulphate;
   c) Tergitol 4;
   d) Sodium selenite;
   e) magnesium chloride;
   f) crystal violet;
   g) bismuth ammonium citrate; and
   h) acid fuschin.
28) A culture medium for the selective growth of Gram negative cells such as Salmonella cells or E. coli cells, optionally the growth of Gram negative cells such as Salmonella cells or E. coli cells that may be present in a test sample, comprising an agent of Formula I as defined in any of embodiments 1-5 and at least one further agent.
29) The culture medium according to embodiment 28 wherein the agent of Formula I is present in the culture media in a concentration of:
a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L; and/or
b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L; or
c) 5 mg/L.
30) The culture medium according to any of embodiments 28 or 29 wherein the agent of Formula I is present in the culture media in a concentration of around 5 mg/L, optionally 5 mg/L.
31) The culture medium according to any one of embodiments 28-30 wherein the culture medium further comprises one, two, three or four or more anti-bacterial agents, optionally comprises Cefsulodin and/or Novobiocin.
31a) The culture medium according to any one of embodiments 1-31 wherein the culture medium further comprises ferric chloride and/or a sulphite compound such as potassium sulphite.
32) The culture medium according to any of embodiments 28-31 wherein the culture medium further comprises an agent that inhibits the growth of, or kills, Pseudomonas aeruginosa, and/or other pseudomonads, optionally comprises Cefsulodin.
33) The culture medium according to any of embodiments 28-32 wherein the culture medium further comprises Cefsulodin at a concentration of
   between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L;
   optionally 8 mg/L.
34) The culture medium according to any one of embodiments 28-33, wherein the culture medium comprises novobiocin in an amount of:
   between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   10 mg/L.
34a) The method of any one of embodiments 1-27 or the culture medium of any of embodiments 28-34, wherein the culture medium comprises ferric chloride in an amount of:
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/.L
34b) The method of any one of embodiments 1-27 or the culture medium of any of embodiments 28-34a, wherein the culture medium comprises a sulphite compound such as potassium sulphite in an amount of:
   a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
   c) at least 0.5 g/L. 075 g/L, 1.00 g/L, 1.25 g/L, 1.50 g/L, 175 g/L, 2.00 g/L, 2.25 g/L, 2.50 g/L, 2.75 g/L, 3.0 g/L, 3.5 g/L, or 4.0 g/L; or
   d) 1.00 g/L; or
   e) 1.11 g/L.
35) The culture medium of any one of embodiments 28-34b, wherein the culture medium comprises novobiocin at a concentration of around 10 mg/L, optionally 10 mg/L; and also comprises Cefsulodin at a concentration of around 8 mg/L, optionally 8 mg/L; and optionally comprises ferric chloride at a concentration of 1.33 mg/L; and/or optionally comprises a sulphite compound such as potassium sulphite at a concentration of 1.00 g/L.
36) The culture medium of any of embodiments 28-35, wherein the culture medium comprises buffered peptone water.
37) The culture medium of any of embodiments 28-36 wherein the culture medium comprises a base broth selected from the group consisting of peptone, tryptone, nutrient broth, L-broth, Gram negative broth, tryptic soy broth with yeast, modified tryptic soy broth and buffered peptone water.
38) The culture medium of any one of embodiments 28-37 wherein the culture medium does not comprise any one of, any two or, or all of:
   a) vancomycin;
   b) Brilliant green; and
   c) Malachite green.
39) The culture medium of anyone of embodiments 28-38 wherein the culture medium does not comprise any one of, any two or more of, or all of:
   a) bile salts, optionally deoxycholate;
   b) Sodium thiosulphate;
   c) Tergitol 4;
   d) Sodium selenite;
   e) magnesium chloride;
   f) crystal violet;
   g) bismuth ammonium citrate; and
   h) acid fuschin.
40) The culture medium according to any of embodiments 28-39 wherein the culture medium is a liquid culture medium.
41) The culture medium according to any of embodiments 28-39 wherein the culture medium is a solid or gel culture medium, optionally comprises agar.
41a) A culture medium comprising ferric chloride and a sulphite compound, optionally potassium sulphite, optionally wherein the concentration of ferric chloride is
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/L and the concentration of the sulphite compound, optionally potassium sulphite, is
   a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
   c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
   d) 1.00 g/L; or
   e) 1.11 g/L.
41b) A lyophilised culture medium wherein the culture medium comprises ferric chloride and a sulphite compound optionally potassium sulphite, wherein the concentration of ferric chloride and the sulphite compound is such that upon resuspension the concentration of ferric chloride is:
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L; 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/L and the concentration of the sulphite compound, optionally potassium sulphite, is
   a) between around 0.5 g/L and 4 g/L, for example between 0.75 g/L and 3.5 g/L; 1.0 g/L and 3.0 g/L; 1.25 g/L and 2.75 g/L; 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 200 g/L, 175 g/L, 1.50 g/L, 1.25 g/L, 1.00 g/L, 0.75 g/L, or 0.50 g/L; and/or
   c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
   d) 1.00 g/L; or
   e) 1.11 g/L;
   optionally wherein resuspension of the lyophilised powder occurs at between 0.1 g/L and 10 g/L, or between 0.5 g/L and 5 g/L, 1 g/L and 3 g/L.
42) A composition for use in preparing a culture medium according to any of embodiments 28-41b.
43) A composition comprising an agent of Formula I as defined in any of embodiments 1-5 and at least one further agent selected from the group comprising Cefsulodin and Novobiocin.
44) The composition according to any of embodiments 42 or 43 wherein the composition comprises Cefsulodin and Novobiocin.
44a) The composition according to any of embodiments 42-44 wherein the composition comprises ferric chloride and/or a sulphite compound such as potassium sulphite.
45) The composition according to any of embodiments 42-44 wherein the concentration of the agent of Formula I, and/or Novobiocin, and/or Cefsulodin, and/or ferric chloride, and/or a sulphite compound such as potassium sulphite is a concentrated concentration such that following dilution the resultant concentration of the:
i) agent of Formula I is:
   a) 0.5 mg/L or more than 0.5 mg/L, for example at least 0.75 mg/L, for example at least 1.0 mg/L, or at least 1.25 mg/L, or at least 1.50 mg/L, or at least 1.75 mg/L, or at least 2.0 mg/L, or at least 2.25 mg/L, or at least 2.50 mg/L, or at least 2.75 mg/L, or at least 3.0 mg/L, or at least 3.25 mg/L, or at least 3.5 mg/L, or at least 3.75 mg/L, or at least 4.0 mg/L, or at least 4.25 mg/L, or at least 4.50 mg/L, or at least 4.75 mg/L, or at least 5.0 mg/L, or at least 5.25 mg/L, or at least 5.75 mg/L, or at least 6.0 mg/L, or at least 6.25 mg/L, or at least 6.50 mg/L, or at least 6.75 mg/L, or at least 7.0 mg/L, or at least 7.25 mg/L, or at least 7.50 mg/L, or at least 7.75 mg/L, or at least 8.0 mg/L, or at least 8.25 mg/L, or at least 8.5 mg/L, or at least 8.75 mg/L, or at least 9.0 mg/L, or at least 9.25 mg/L, or at least 9.50 mg/L, or at least 9.75 mg/L, or at least 10.00 mg/L, or at least 11 mg/L, or at least 12 mg/L, or at least 13 mg/L, or at least 14 mg/L, or at least 15 mg/L; and/or
   b) less than 15 mg/L, for example less than 14 mg/L, or less than 13 mg/L, or less than 12 mg/L, or less than 11 mg/L, or less than 10.00 mg/L, or less than 9.75 mg/L, or less than 9.50 mg/L, or less than 9.25 mg/L, or less than 9.0 mg/L, or less than 8.75 mg/L, or less than 8.5 mg/L, or less than 8.25 mg/L, or less than 8.0 mg/L, or less than 7.75 mg/L, or less than 7.50 mg/L, or less than 7.25 mg/L, or less than 7.0 mg/L, or less than 6.75 mg/L, or less than 6.50 mg/L or less than 6.25 mg/L, or less than 6.0 mg/L, or less than 5.75 mg/L or less than 5.25 mg/L, or less than 5.0 mg/L, or less than 4.75 mg/L, or less than 4.50 mg/L, or less than 4.25 mg/L, or less than 4.0 mg/L, or less than 3.75 mg/L, or less than 3.5 mg/L, or less than 3.25 mg/L or less than 3.0 mg/L, or less than 2.75 mg/L, or less than 2.50 mg/L, or less than 2.25 mg/L, or less than 2.0 mg/L, or less than 1.75 mg/L, or less than 1.50 mg/L, or less than 1.25 mg/L, or less than 1.0 mg/L; or
   c) 5 mg/L
ii) Novobiocin is:
   a) between 40 mg/L and 2 mg/L, optionally between 35 mg/L and 5 mg/L, optionally between 30 mg/L and 7 mg/L, optionally between 25 mg/L and 9 mg/L, optionally between 20 mg/L and 11 mg/L, optionally between 15 mg/L and 12 mg/L, optionally 10 mg/L or 11 mg/L or 12 mg/L or 13 mg/L or 14 mg/L or 15 mg/L; and/or
   b) at least 2 mg/L, or at least 3 mg/L, optionally at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 35 mg/L, optionally less than 30 mg/L, or less than 25 mg/L, or less than 22 mg/L, less than 20 mg/L, less than 19 mg/L, less than 18 mg/L, less than 17 mg/L, less than 16 mg/L, less than 15 mg/L, less than 14 mg/L, less than 13 mg/L, less than 12 mg/L, less than 11 mg/L, less than 10 mg/L, less than 9 mg/L, less than 8 mg/L, less than 7 mg/L, less than 6 mg/L, less than 5 mg/L, less than 4 mg/L, less than 3 mg/L, less than 2 mg/L or less than 1 mg/L; or
   10 mg/L;
iii) Cefsulodin is:
   a) between 1 mg/L and 20 mg/L, optionally between 2 mg/L and 19 mg/L, optionally between 3 mg/L and 18 mg/L, optionally between 4 mg/L and 17 mg/L, optionally 5 mg/L and 16 mg/L, optionally between 6 mg/L and 15 mg/L, optionally between 7 mg/L and 14 mg/L, optionally between 8 mg/L and 13 mg/L, optionally between 9 mg/L and 12 mg/L, optionally between 10 mg/L and 11 mg/L, optionally at a concentration of around 8 mg/L, optionally 8 mg/L; and/or
   b) at least 1 mg/L, or at least 2 mg/L, or at least 3 mg/L, or at least 4 mg/L, or at least 5 mg/L, or at least 6 mg/L, or at least 7 mg/L, or at least 8 mg/L, or at least 9 mg/L, or at least 10 mg/L, or at least 11 mg/L, or at least 12 mg/L, or at least 13 mg/L, or at least 14 mg/L, or at least 15 mg/L, or at least 16 mg/L, or at least 17 mg/L, or at least 18 mg/L, or at least 19 mg/L, or at least 20 mg/L; and/or
   c) less than 20 mg/L, or less than 19 mg/L, or less than 18 mg/L, or less than 17 mg/L, or less than 16 mg/L, or less than 15 mg/L, or less than 14 mg/L, or less than 13 mg/L, or less than 12 mg/L, or less than 11 mg/L, or less than 10 mg/L, or less than 9 mg/L, or less than 8 mg/L, or less than 7 mg/L, or less than 6 mg/L, or less than 5 mg/L, or less than 4 mg/L, or less than 3 mg/L, or less than 2 mg/L, or less than 1 mg/L;
iv) Ferric chloride is:
   a) between around 0.5 mg/L and 4 mg/L, for example between 0.75 mg/L and 3.5 mg/L; 1.0 mg/L and 3.0 mg/L; 1.25 mg/L and 2.75 mg/L; 1.50 mg/L and 2.50 mg/L: 1.75 mg/L and 2.25 mg/L; or 2.00 mg/L; and/or
   b) is less than 4 mg/L, 3.5 mg/L, 3.0 mg/L, 2.75 mg/L, 2.50 mg/L, 2.25 mg/L, 2.00 mg/L, 1.75 mg/L, 1.50 mg/L, 1.25 mg/L, 1.00 mg/L, 0.75 mg/L, 0.50 mg/L or 0.25 mg/L; and/or
   c) at least 0.25 mg/L, 0.50 mg/L, 0.75 mg/L, 1.00 mg/L, 1.25 mg/L, 1.50 mg/L, 1.75 mg/L, 2.00 mg/L, 2.25 mg/L, 2.50 mg/L, 2.75 mg/L, 3.00 mg/L, 3.25 mg/L, 3.75 mg/L, 4.00 mg/L; or
   d) 1.33 mg/L; or
   e) 1.37 mg/L
      and/or
v) a sulphite compound such as Potassium sulphite is:
   a) between around 0.5 g/L and 4 g/L, for example between 0.75 g/L and 3.5 g/L; 1.0 g/L and 3.0 g/L; 1.25 g/L and 2.75 g/L: 1.50 g/L and 2.50 g/L; 1.75 g/L and 2.25 g/L; or 2.00 g/L; and/or
   b) less than 4.00 g/L, 3.5 g/L, 3.0 g/L, 2.75 g/L, 2.50 g/L, 2.25 g/L, 2.00 g/L, 1.75 g/L, 1.50 g/L, 1.25 g/L, 1.00 g/L, 0.75 g/L, or 0.50 g/L; and/or
   c) at least 0.5 g/L, 0.75 g/L, 1.00 g/L, 1.25 g/L, 1.50 g/L, 1.75 g/L, 2.00 g/L, 2.25 g/L, 2.50 g/L, 2.75 g/L, 3.0 g/L, 3.5 g/L, or 4.0 g/L; or
   d) 100 g/L; or
   e) 1.11 g/L.
46) The composition according to any of embodiments 42-45 wherein the concentration of:
a) the agent of Formula I is between 1.00 mg/ml and 1.50 mg/ml, for example between 1.10 mg/ml and 1.40 mg/ml, for example between 1.20 mg/ml and 1.30 mg/ml, optionally wherein the concentration is 1.125 mg/ml, for example wherein the agent of Formula I is dissolved in 70% v:v ethanol;
b) the Novobiocin is between 2.00 mg/ml and 3.00 mg/ml, for example between 2.10 mg/ml and 2.90 mg/ml, for example between 2.20 mg/ml and 2.80 mg/ml, for example between 2.30 mg/ml and 2.70 mg/ml, for example between 2.40 mg/ml and 2.60 mg/ml, for example 2.50 mg/ml, for example the concentration may be 2.7 mg/ml or 2.25 mg/ml, preferably 2.25 mg/ml, for example wherein the novobiocin is dissolved in 70% v/v ethanol; and
c) the Cefsulodin is between 1.4 mg/ml and 2.0 mg/ml, for example between 1.5 mg/ml and 1.9 mg/ml, for example between 1.6 mg/ml and 1.8 mg/ml, for example between 1.7 mg/ml and 1.7 mg/ml, optionally wherein the concentration is 1.8 mg/ml, optionally wherein the Cefsulodin is dissolved in 70% v/v ethanol;
d) Ferric chloride is between around 10 mg/L to 10 g/L, for example between 100 mg/L and 5 g/L, 1 g/L and 3 g/L;
   and/or
e) a sulphite compound such as Potassium sulphite is
   between around 5 g/L and 50 g/L, for example between 10 g/L and 40 g/L, or 20 g/L and 30 g/L.
47) The composition according to any of embodiments 42-46 wherein the composition is lyophilised.
48) A method for quantifying or detecting the presence or absence of target Gram negative bacteria, optionally for quantifying or detecting the presence or absence of Salmonella or E. coli in a test sample, the method comprising selectively culturing microbial cells present in the test sample according to any of embodiments 1-27 and quantifying or detecting the presence or absence of target Gram negative cells, optionally quantifying or detecting the presence or absence of Salmonella or E. coli.
49) The method of embodiment 48 wherein the test sample is:
   a food product, optionally meat, meat products including mince, eggs, cheese, milk, vegetables, chocolate, confectionery, peanut butter and the like including processed, dried, frozen or chilled food products, a spice, a herb or a flavouring product;
   a clinical sample such as a biopsy sample, faecal, saliva, hydration fluid, nutrient fluid, blood, blood product, tissue extract, vaccine, anaesthetic, pharmacologically active agent, imaging agent or urine sample and the like;
   a swab, for example a skin swab, a caecum swab, a faecal swab, a cloaca swab or a rectal swab; a swab of surfaces such as floors, doors and walls; a swab of food products including animal carcass swabs;
   a cosmetic sample such as foundation makeup, lip-balms, lotions, creams, shampoos and the like;
   a sample of a liquid in which a sample such as food or a swab has been suspended; or
   a sample of raw meat or raw poultry, or a swab taken from raw meat or raw poultry.
50) The method of any of embodiments 48 and 49 wherein the cultured microbial cells are treated so as to cause release of bacterial LPS and/or core oligosaccharide from any microorganism present within the test sample,
   optionally where said treating comprises heating the microbial cells, optionally heating to about 60° C. to about 100° C., optionally 65° C. to 100° C., optionally 70° C. to 100° C., optionally 75° C. to 100° C., optionally 80° C. to 100° C., optionally 85° C. to 100° C.,
   optionally 90° C. to 100° C., optionally 95° C. to 100° C., optionally wherein the cultured microbial cells are heated for about 30 seconds to about 30 minutes, optionally about 2, 3, 4, 5, 6, 7, 8, 9, 10, 11, 12, 13, 14, 15, 16, 17, 18, 19, 20, 21, 22, 23, 24 or about 25 minutes, optionally 15 to 20 minutes.
51) The method according to any of embodiments 48-50 wherein said quantifying and/or detecting comprises exposing the cultured microbial cells or the bacterial LPS and/or core oligosaccharide of the treated cultured microbial cells to a capture member, resulting in captured cells or captured bacterial LPS and/or core oligosaccharide,
   optionally wherein said exposing occurs after the cultured microbial cells are treated so as to cause release of bacterial LPS and/or core oligosaccharide from any microorganism present within the test sample.
52) The method according to embodiment 51 wherein the capture member is an antibody or antibody fragment, optionally a biotinylated antibody or antibody fragment,
   optionally wherein the capture member is a sheep polyclonal antibody, raised against Salmonella.
53) The method according to any of embodiments 51 and 52 wherein the capture member is immobilised to a substrate or solid surface, optionally immobilised to a substrate or solid surface via an intermediate binding partner, optionally wherein the intermediate binding partner is a linkage comprising biotin and streptavidin.
54) The method according to any of embodiments 51-53 wherein the capture member is specific for microorganisms, optionally specific for bacteria, optionally specific for Gram negative bacteria, optionally specific for a target Gram negative bacterial species.
55) The method according to any of embodiments 51-54 wherein the capture member has specificity for Salmonella or E. coli
56) The method according to any of embodiments 51-54 wherein the capture member is selected from the group consisting of
   a monoclonal antibody with specificity for the LPS or core oligosaccharide of Salmonella, optionally 3D11, G7, M181 or T6 antibody
   ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS; or
      Salmonella typhi 6301, 6321, 6345, 6331, 6347.
57) The method according to any of embodiments 48 to 56 further comprising exposing the captured cells or captured bacterial LPS and/or core oligosaccharide to a detection member, optionally wherein said exposing to a detection member occurs after the captured cells or captured bacterial LPS/and/or core oligosaccharide is exposed to a wash solution, optionally wherein the wash solution comprises:
   a) monovalent ions wherein the molar concentration of monovalent ions and/or salts in the wash solution is at least 100, 137, 150, 200, 250, 300, 350, 400, 450, 500, 550, 600 or 650 mM, or between 100 and 700 mM or 750 mM or 800 mM, or between 300 and 650 mM, optionally wherein the molar concentration of monovalent ions and/or salts present in the wash solution is at least 200 mM, 300 mM, 400 mM, 500 mM, 600 mM, 700 or 800 mM optionally 471 mM, 513.3 mM or 599 mM.
   optionally wherein the monovalent ion and/or salt in the wash solution is a lithium, sodium or potassium ion and/or salt, optionally wherein the monovalent salt is LiCl, NaCl and/or KCl;
      or
   b) divalent ions and/or salts wherein the molar concentration of divalent ions and/or salts in the wash solution is between 1 and 30 mM, optionally between 5 and 15 mM, between 12 and 14 mM or between 5 and 8 mM, optionally 6 to 12 mM, optionally 8.4 mM, 6.6 mM, 13 mM or 26 mM,
   optionally wherein the divalent ion and/or salt in the wash solution in step (vi) is a magnesium or calcium ion and/or salt, optionally wherein the divalent salt is MgCl₂ and/or CaCl₂.
58) The method according to embodiment 57 wherein the detection member binds to the captured cells or captured bacterial LPS and/or core oligosaccharide to form a detection complex, optionally wherein the detection member is an antibody or antibody fragment.
59) The method according to any of embodiments 57 and 58 wherein the detection member is specific for microorganisms, optionally specific for bacteria, optionally specific for Gram negative bacteria.
60) The method according to any of embodiments 57 to 59 wherein the detection member has specificity for the target Gram negative bacterial species, optionally specificity for Salmonella or E. coli.
61) The method according to any of embodiments 57-60 wherein the detection member is an antibody selected from the group consisting of:
   a monoclonal antibody with specificity for the LPS or core oligosaccharide of Salmonella, optionally 3D11, G7, M181 or T6 antibody
   ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS; or
      Salmonella typhi 6301, 6321, 6345, 6331, 6347.
62) The method according to any one of embodiments 57-61 wherein said exposing to said detection member is performed at a molar concentration of monovalent ions and/or salts that is:
   a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
   b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
   c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
   optionally wherein the monovalent ion and/or salt is a lithium, sodium and/or potassium ion and/or salt, optionally wherein the monovalent salt is LiCl, NaCl and/or KCl.
63) The method according to any of embodiments 57-62 wherein the method further comprises exposing the detection complex to a wash solution comprising:
   a) monovalent ions wherein the molar concentration of monovalent ions and/or salts in the wash solution is at least 100, 137, 150, 200, 250, 300, 350, 400, 450, 500, 550, 600 or 650 mM, or between 100 and 700 mM or 750 mM or 800 mM, or between 300 and 650 mM, optionally wherein the molar concentration of monovalent ions and/or salts present in the wash solution is at least 200 mM, 300 mM, 400 mM, 500 mM, 600 mM, 700 or 800 mM optionally 471 mM, 513.3 mM or 599 mM.
   optionally wherein the monovalent ion and/or salt in the wash solution is a lithium, sodium or potassium ion and/or salt, optionally wherein the monovalent salt is LiCl, NaCl and/or KCl:
      or
   b) divalent ions and/or salts wherein the molar concentration of divalent ions and/or salts in the wash solution is between 1 and 30 mM, optionally between 5 and 15 mM, between 12 and 14 mM or between 5 and 8 mM, optionally 6 to 12 mM, optionally 8.4 mM, 6.6 mM, 13 mM or 26 mM,
   optionally wherein the divalent ion and/or salt in the wash solution in step (vi) is a magnesium or calcium ion and/or salt, optionally wherein the divalent salt is MgCl₂ and/or CaCl₂).
64) The method according to any of embodiments 57-63 wherein said detection of the presence or absence of the target Gram negative bacteria, optionally Salmonella or E. coli, or the quantification of the target Gram negative bacteria, optionally Salmonella or E. coli comprises detection of the presence or absence of the detection member, optionally wherein said detection of the presence or absence of the detection member occurs after exposure to a wash solution according to embodiment 60.
65) The method according to embodiment 64 wherein said detection of the presence or absence of the detection member comprises exposure to an antibody with specificity for the detection member, optionally wherein the antibody is an enzyme-conjugated antibody, optionally a HRP-conjugated antibody.
66) The method according to embodiment 65 wherein the enzyme results in an observable colour change, optionally results in an observable colour change upon addition of the enzyme substrate.
67) The method according to any of embodiments 64 to 66 wherein detection of the presence of the detection complex indicates the presence of the target Gram negative bacteria, optionally Salmonella or E. coli in the initial test sample.
68) A method for quantifying or detecting the presence or absence of a target Gram negative bacterial species, optionally Salmonella or E. coli in a test sample wherein the method comprises:
   a) culturing the microbial cells in the test sample, optionally wherein the microbial cells are cultured according to the method of any one of embodiments 1-27;
   b) treating the cultured microbial cells so as to cause release of bacterial LPS and/or core oligosaccharide from any microorganism present within the test sample;
   c) exposing the cultured cells or the bacterial LPS and/or core oligosaccharide of the treated cultured test sample to a capture member, resulting in captured cells or captured bacterial LPS and/or core oligosaccharide, optionally wherein the capture member is a sheep polyclonal antibody, raised against Salmonella;
   d) exposing the captured cells or captured bacterial LPS and/or core oligosaccharide to a detection member to form a detection complex wherein the detection member is an antibody with specificity for the target Gram negative bacterial species, optionally specificity for Salmonella or E. coli, wherein said exposing to said detection member is performed at a molar concentration of monovalent ions and/or salts that is
      i) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
      ii) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
      iii) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
      optionally wherein the monovalent ion and/or salt is a lithium, sodium and/or potassium ion and/or salt, optionally wherein the monovalent salt is LiCl, NaCl and/or KCl.
   e) exposing the detection complex to a wash solution comprising:
      i) monovalent ions wherein the molar concentration of monovalent ions and/or salts in the wash solution is at least 100, 137, 150, 200, 250, 300, 350, 400, 450, 500, 550, 600 or 650 mM, or between 100 and 700 mM or 750 mM or 800 mM, or between 300 and 650 mM; or
      ii) divalent ions and/or salts wherein the molar concentration of divalent ions and/or salts in the wash solution is between 1 and 30 mM, optionally between 5 and 15 mM;
   f) detecting the presence or absence of the detection complex, optionally wherein said detecting comprises exposure of the detection complex to an enzyme-conjugated antibody with specificity for the detection member, optionally a HRP-conjugated antibody,
      wherein detection of the presence of the detection complex indicates the presence of the target Gram negative bacterial species, optionally Salmonella or E. coli in the initial test sample.
69) A kit for the selective culture of Gram negative bacteria such as Salmonella cells or E. coli cells, optionally Gram negative bacteria cells such as Salmonella cells or E. coli cells that may be present in a test sample, optionally wherein the culture is performed according to any of embodiments 1-27, wherein the kit comprises:
   an agent of Formula I as defined in any of embodiments 1-5; and
   Cefsulodin and/or novobiocin;
   optionally wherein the agent of Formula I is 4PYcq.
69a) A kit for the selective culture of Gram negative bacteria cells such as Salmonella cells or E. coli cells, optionally Gram negative bacteria cells such as Salmonella cells or E. coli cells that may be present in a test sample, optionally wherein the culture is performed according to any of embodiments 1-27, wherein the kit comprises:
a) an agent of Formula I as defined in the first aspect of the disclosure (a); and any one or more of Cefsulodin (b), novobiocin (b), ferric chloride (1) or a sulphite compound such as potassium sulphite (m), optionally wherein the agent of Formula I is 4PYcq;
b) an agent of Formula I as defined in the first aspect of the disclosure (a), and Cefsulodin (b), and novobiocin (b) and optionally ferric chloride (1) and/or a sulphite compound such as potassium sulphite (m), optionally wherein the agent of Formula I is 4PYcq; or
c) an agent of Formula I as defined in the first aspect of the disclosure (a), and Cefsulodin (b), and novobiocin (b) and ferric chloride (1) and a sulphite compound such as potassium sulphite (m),
   optionally wherein the agent of Formula I is 4PYcq.
69a) A kit for the selective culture of Gram negative bacteria cells such as Salmonella cells or E. coli cells, optionally Gram negative bacteria cells such as Salmonella cells or E. coli cells that may be present in a test sample, optionally wherein the culture is performed according to any of embodiments 1-27, wherein the kit comprises:
   first composition that comprises an agent of Formula I optionally 4PYcq (a), Cefsulodin (b) and Novobiocin (c);
      and further comprises a second composition that comprises ferric chloride and a sulphite compound such as potassium sulphite, optionally wherein the second composition is a culture media, optionally is a lyophilised culture media that comprises the ferric chloride and the sulphite compound such as potassium sulphite.
70) A kit for use in a method of quantifying or detecting the presence or absence of a target Gram negative bacterial cell, optionally Salmonella or E. coli, optionally for quantifying or detecting the presence or absence of a target Gram negative bacterial cell optionally Salmonella or E. coli in a test sample, optionally wherein the method of quantifying or detecting the presence or absence of a target Gram negative bacterial cell optionally Salmonella or E. coli is performed according to any of embodiments 48-68, wherein the kit comprises any two or more of:
a) an agent of Formula I as defined in any of embodiments 1-5, optionally wherein the agent of Formula I is 4PYcq;
b) Cefsulodin and/or novobiocin;
c) an agent to treat microbial cells so as to cause release of bacterial LPS and/or core oligosaccharide from any microorganism present, optionally alcohol or a water based buffer;
d) a capture member,
   optionally wherein the capture member is an antibody or antibody fragment, optionally a biotinylated antibody or antibody fragment,
   optionally wherein the capture member is specific for microorganisms, optionally specific for bacteria, optionally specific for Gram negative bacteria, optionally specific for Salmonella, optionally wherein the capture member is a sheep polyclonal antibody raised against Salmonella
      optionally wherein the capture member is immobilised to a substrate or solid surface, optionally immobilised to a substrate or solid surface via an intermediate binding partner, optionally wherein the intermediate binding partner is a linkage comprising biotin and streptavidin;
e) a detection member, optionally
   wherein the detection member is an antibody or antibody fragment; and/or
   wherein the capture member is specific for microorganisms, optionally specific for bacteria, optionally specific for Gram negative bacteria, optionally specific for the target Gram negative bacteria species, optionally specific for Salmonella or E. coli, and/or
   wherein the detection member binds to the captured cells or captured bacterial LPS and/or core oligosaccharide to form a detection complex; and/or
   wherein the detection member is an antibody selected from the group consisting of
   3D11, G7, M181 or T6
   or ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS;
      Salmonella typhi 6301, 6321, 6345, 6331, 6347;
f) a wash solution comprising monovalent ions wherein the molar concentration of monovalent ions and/or salts in the wash solution is at least 100, 137, 150, 200, 250, 300, 350, 400, 450, 500, 550, 600 or 650 mM, or between 100 and 700 mM or 750 mM or 800 mM, or between 300 and 650 mM;
g) a wash solution comprising divalent ions and/or salts wherein the molar concentration of divalent ions and/or salts in the wash solution is between 1 and 30 mM, optionally between 5 and 15 mM;
h) a culture medium as defined in any of embodiments 48-53;
i) an antibody with specificity for the detection member, optionally wherein the antibody is an enzyme-conjugated antibody, optionally a HRP-conjugated antibody.
j) streptavidin assay plates, wherein the inner surface of the wells is coated in biotinylated polyclonal antibodies with specificity for Salmonella or microbial fragments thereof;
k) instructions for performing the method of the first, second and third aspects of the disclosure;
l) ferric chloride; and/or
m) potassium sulphide.
71) A method of diagnosing a subject as being infected with a target Gram negative bacterial species, optionally infected with Salmonella or E. coli wherein the method comprises culturing the microorganisms in a test sample according to any of embodiments 1-27 and/or detecting the presence or absence of the target Gram negative bacterial species, optionally Salmonella or E. coli according to the method of any one of embodiments 48-68.
72) An anti-Gram negative bacteria agent, optionally an anti-Salmonella or anti-E. coli agent for use in treating a subject diagnosed as infected with a target Gram negative bacterial species, optionally infected with Salmonella or E. coli wherein the diagnosis is performed according to embodiment 71.
73) A method for the detection of the presence of a target Gram negative bacterial species, optionally Salmonella or E. coli in a sample comprising or expected to comprise microbial cells, optionally wherein the sample is a sample of cultured microbial cells,
   wherein the method comprises an immunoassay wherein the immunoassay comprises the use of a detection member, wherein the sample or microbial cells are exposed to the detection member at a salt concentration of:
      a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
      b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
      c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
      optionally wherein the monovalent ion and/or salt is a lithium, sodium and/or potassium ion and/or salt, optionally wherein the monovalent salt is LiCl, NaCl and/or KCl.
74) A method for the detection of the presence of a target Gram negative bacterial species, optionally Salmonella or E. coli in a sample comprising or expected to comprise microbial cells, optionally wherein the sample is a sample of cultured microbial cells, wherein the method comprises an immunoassay wherein the immunoassay comprises:
   a) optionally treating the sample or cultured microbial cells so that the microbial cells are treated so as to cause release of bacterial LPS and/or core oligosaccharide from any microorganism present within the test sample;
   b) exposing the sample or the cultured microbial cells or the bacterial LPS and/or core oligosaccharide of the treated cultured microbial cells to a capture member, resulting in captured cells or captured bacterial LPS and/or core oligosaccharide,
      optionally wherein said exposing occurs after the cultured microbial cells are treated so as to cause release of bacterial LPS and/or core oligosaccharide from any microorganism present within the test sample, optionally
   wherein the capture member is an antibody or antibody fragment, optionally a biotinylated antibody or antibody fragment, optionally wherein the capture member has specificity for the target Gram negative bacteria, optionally specificity for Salmonella or E. coli, optionally wherein the capture member is a sheep polyclonal antibody, raised against Salmonella,
   optionally wherein the capture member is
      a monoclonal antibody with specificity for the LPS or core oligosaccharide of Salmonella, optionally 3D11, G7, M181 or T6;
   ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS; or
      Salmonella typhi 6301, 6321, 6345, 6331, 6347; and
   c) exposing the captured cells or captured bacterial LPS and/or core oligosaccharide to a detection member, wherein the detection member binds to the captured cells or captured bacterial LPS and/or core oligosaccharide to form a detection complex, optionally wherein the detection member is an antibody or antibody fragment, optionally wherein the detection member has specificity for the target Gram negative bacteria, optionally specificity for Salmonella or E. coli, optionally wherein the detection member is selected from the group consisting of:
      a monoclonal antibody with specificity for the LPS or core oligosaccharide of Salmonella, optionally 3D11, G7, M181 or T6;
   ViroStat product number:
      Salmonella sp. 6371-6394, 6301-6347, 6301-flagella, 6321-flagella, 6331-typhimurium, 6392-common core, 6393, 6394, 6391, 6381, 6371;
      Salmonella paratyphi A 6347-LPS; or
      Salmonella typhi 6301, 6321, 6345, 6331, 6347;
         and
   wherein the captured cells or captured bacterial LPS and/or core oligosaccharide are exposed to the detection member at a salt concentration of:
      a) no more than 200 mM, optionally less than 200 mM, optionally less than 175 mM, optionally less than 150 mM, optionally less than 125 mM, optionally less than 100 mM, optionally less than 90 mM, optionally less than 80 mM, optionally less than 70 mM, optionally less than 60 mM, optionally less than 50 mM, optionally less than 40 mM, optionally less than 35 mM, optionally less than 30 mM, optionally less than 25 mM, optionally less than 20 mM, optionally less than 15 mM, optionally less than 10 mM, optionally less than 5 mM; and/or
      b) between 5 mM and 200 mM, optionally between 10 mM and 190 mM, optionally between 15 mM and 180 mM, optionally between 20 mM and 170 mM, optionally between 25 mM and 160 mM, optionally between 30 mM and 150 mM, optionally between 35 mM and 140 mM, optionally 40 m and 130 mM, optionally between 45 mM and 120 mM, optionally between 50 mM and 110 mM, optionally between 55 mM and 100 mM, optionally between 60 mM and 90 mM, optionally between 70 and 80 mM;
      c) between 5 mM and 30 mM, optionally between 6 mM and 29 mM, optionally between 7 mM and 28 mM, optionally between 8 mM and 27 mM, optionally between 9 mM and 26 mM, optionally between 10 mM and 25 mM, optionally between 11 mM and 24 mM, optionally between 12 mM and 23 mM, optionally between 13 mM and 22 mM, optionally between 14 mM and 21 mM, optionally between 15 mM and 20 mM, optionally between 16 mM and 19 mM, optionally between 17 mM and 18 mM, optionally 20 mM,
      optionally wherein the monovalent ion and/or salt is a lithium, sodium and/or potassium ion and/or salt, optionally wherein the monovalent salt is LiCl, NaCl and/or KCl.

### FIGURE LEGENDS

FIG. 1

FIG. 1 illustrates the general structure of the LPS (O-antigen, core polysaccharide (oligosaccharide), lipid A) of certain bacteria of interest and shows a detailed illustration of the Salmonella LPS monomer including the species specific antibody binding epitope.

FIG. 2—ELISA values obtained from the primary enrichment broth for both the ISO test and the test of the present disclosure.

FIG. 2 illustrates ELISA buffered peptone water optical densities (450 nm) generated from various food samples artificially inoculated with low levels of multiple Salmonella serovars enriched using either an ISO 2-step enrichment protocol or a cefsulodin, novobiocin, 4PYcq lyophilised single step culture at 41.5° C., where an OD₍₄₅₀₎≥0.20 is a presumptive positive result. The ELISA readings in both cases represent BPW samples taken at 20 hours and after heat treatment tested in our preferred ELISA system. Note the improved optical densities with the novel selective enrichment step compared to un-supplemented BPW.

Supplements (Abtek RK26S and SB29S) 1 ml added to 225 ml of BPW. Supplemented BPW @ 41.5° C. (20 hours). Food samples inoculated and left at 4 C for 48 hours before addition of media/stomaching/incubation at 41.5 C for 20 hours.

FIG. 3

FIG. 3 illustrates internal Method Comparison study data in multiple food categories and sample matrices containing competing microflora. In this unpaired study these samples were artificially inoculated with multiple Salmonella serovars (1-5 colony forming units) and enriched using either an ISO 2-step enrichment protocol; a selective cefsulodin, novobiocin, brilliant green lyophilised single step culture; or a selective cefsulodin, novobiocin, 4PYcq lyophilised single step culture. This data demonstrates the potential inhibitory effect of brilliant green supplement on the growth and recovery of Salmonella even in the presence of food matrixes resulting in an inferior performance to the Reference ISO 2-step enrichment protocol and our alternative supplement with 4PYcq. This is indicated by the 5 negative results seen with the brilliant green supplement compared to ISO and 4PYcq based supplement.

In contrast, within diverse food categories, the 4PYcq supplement shows superior performance over the brilliant green supplement, and performing at least as well as the ISO Reference method.

FIG. 4

FIG. 4 illustrates inclusivity study data where multiple Salmonella serovars were artificially inoculated into either Buffered Peptone Water (BPW) on its own, or BPW enriched with either a selective cefsulodin, novobiocin, brilliant green lyophilised supplement run in the absence or presence of an oatmeal food matrix; or a selective cefsulodin, novobiocin, 4PYcq lyophilised supplement run in the absence of an oatmeal food matrix. This data demonstrates the potentially toxic effect of brilliant green supplement and the requirement of a food matrix to allow growth of the majority of Salmonella serovars. In contrast, the 4PYcq supplement is showing superior performance through the inhibition of Gram positive bacteria thereby allowing the free growth of Salmonella even in the absence of an oatmeal food matrix.

FIG. 5

FIG. 5 illustrates exclusivity study data where multiple non-Salmonella bacterial species were artificially inoculated into Brain Heart Infusion broth at high levels in incubated for 20 hours before testing. This data demonstrates that the Salmonella One candidate test is highly specific and does not cross react with a diverse array of Gram negative bacterial species.

FIG. 6

FIG. 6 illustrates inclusivity study data where multiple Salmonella serovars were artificially inoculated into BPW enriched with a selective cefsulodin, novobiocin, 4PYcq lyophilised supplement at low levels. This data demonstrates that the Salmonella One candidate test is highly specific towards Salmonella and the selective cefsulodin, novobiocin, 4PYcq lyophilised supplement allows the free growth of Salmonella.

FIG. 7

FIG. 7 illustrates internal Method Comparison study data in multiple food categories and sample matrixes containing competing microflora. These samples are artificially inoculated with multiple Salmonella serovars and enriched using either an ISO 2-step enrichment protocol or a selective cefsulodin, novobiocin, 4PYcq lyophilised single step culture. This data demonstrates that within diverse sample matrixes a test according to the present disclosure is performing at least as well as the ISO Reference method, and is allowing the growth of Salmonella to levels high enough to be detected by the candidate test method and alternative cultural confirmation methods. Also in most cases the Salmonella were detected on the primary subcultures from the selective BPW which means basic confirmation of the positive ELISA can be done within 24 hours.

FIG. 8

Recovery of Salmonella from different spice and flavourings in BPW media alone.

FIG. 9

Common spice and flavourings sample analysis—effect of spice neutraliser

FIG. 10

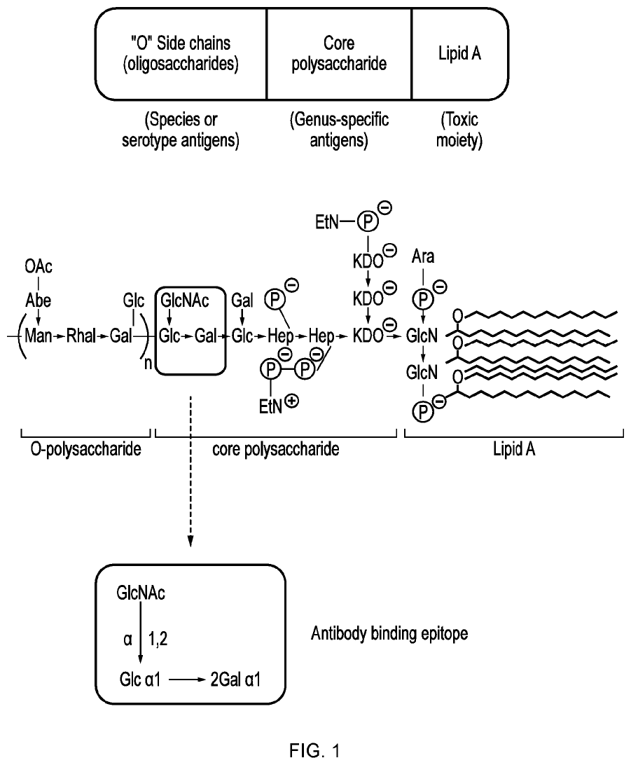

Common spice and flavourings sample analysis—effect of the ‘spice neutraliser’ at fractional inoculation levels

In the presence of the ‘Spice neutraliser’, equivalence is observed between the different BAM reference methods (10-100 g/1:10-1:100 enrichment) and Solus One Salmonella test method (i.e. the one-step culture method) (25 g/1:10 enrichment).

Salmonella weren't detected in Tandoori masala using the BAM reference method, however the Solus Test method with spice neutraliser showed detection—this could be due to experimental variation.

This was at fractional inoculation level (2-8 cfu). Accordingly a method with the ‘Spice neutraliser’ supplement is capable of detecting Salmonella from spice/herb/flavouring samples, even when the Salmonella is present at fractional levels.

FIG. 11

Common spice and flavourings sample analysis—comparison of full strength vs half strength Solus supplement (4PYcq, Novobiocin and Cefsulodin)

Full strength: 5 mg/L 4PYcq, 8 mg/L Cefsulodin and 10 mg/L Novobiocin

Half strength 2.5 mg/L 4PYcq, 4 mg/L Cefsulodin and 5 mg/L Novobiocin

There is equivalence between full strength and half strength Solus supplement.

In the presence of the ‘Spice neutraliser’ supplement, equivalence is observed between the different BAM reference methods (10-100 g/1:10-1:100 enrichment) and the Solus One Salmonella test method (i.e. the one-step culture method) (25 g/1:10 enrichment) Un-spiked samples return low backgrounds. This was at a high inoculation level (40-92 cfu).

FIG. 12

Comparison of single strength vs double strength ‘spice neutraliser’ in the Solus One Salmonella Test method

This data again shows that with half of the strength of the supplement (Half strength 2.5 mg/L 4PYcq, 4 mg/L Cefsulodin and 5 mg/L Novobiocin) plus the “spice neutraliser” (ferric chloride and a sulphite compound such as potassium sulphite), recovery and detection of Salmonella from onion powder, garlic powder and cinnamon is good.

However, again recovery from cloves and oregano is difficult, even in the presence of double strength “spice neutraliser” supplement.

FIG. 13

Relevance of individual spice neutraliser components

FIG. 14

BAM Reference Method

FIG. 15

Validation data, comparing a standard 2-step culture protocol, with a sulphite compound such as potassium sulphite and ferric chloride, to the single-culture method according to the disclosure, also comprising ferric chloride, a sulphite compound such as potassium sulphite and the selective supplement comprising 4PYcq, Novobiocin and Cefsulodin.

FIG. 16

FIG. 17

The chemical structure of some inhibitory components of common herbs and spices.

FIG. 18

Conditions.

FIG. 19

This table shows the growth of stressed Salmonella from non-selective and selective BPW from UHT milk as measured by ELISA. Solus One Salmonella Supplement recovers the Salmonella but inhibits non-Salmonella whereas the Brilliant Green supplement is showing an inhibitory effect.

FIG. 20

Data for the detection of E. coli O157 cultured using methods according to the disclosure. The enrichment for Solus One E. coli O157 uses BPW with the triple supplement (C-X, Novobiocin and Cefsulodin). In this instance, half strength supplement is used (2.22 mL combined supplement per Litre BPW broth, i.e. 2.5 mg/L 4PYcq, 4 mg/L Cefsulodin and 5 mg/L Novobiocin).

The data shows 75% recovery which is fractional recovery. The supplement allows a 2 cfu E. coli O157 inoculum to come through against a 1.68E+08 Gram −ve background.

The disclosure will be further understood with reference to the following non-limiting experimental examples.

### EXAMPLES

### Example 1—Sensitivity of Microbial Cells to Brilliant Green is Influenced by the Presence of Food and the Type of Food

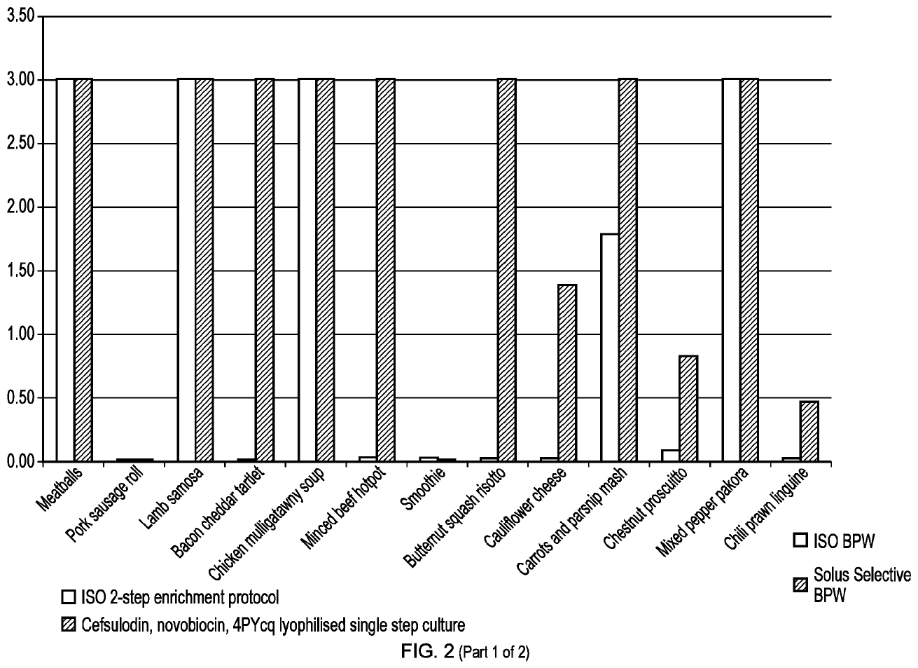

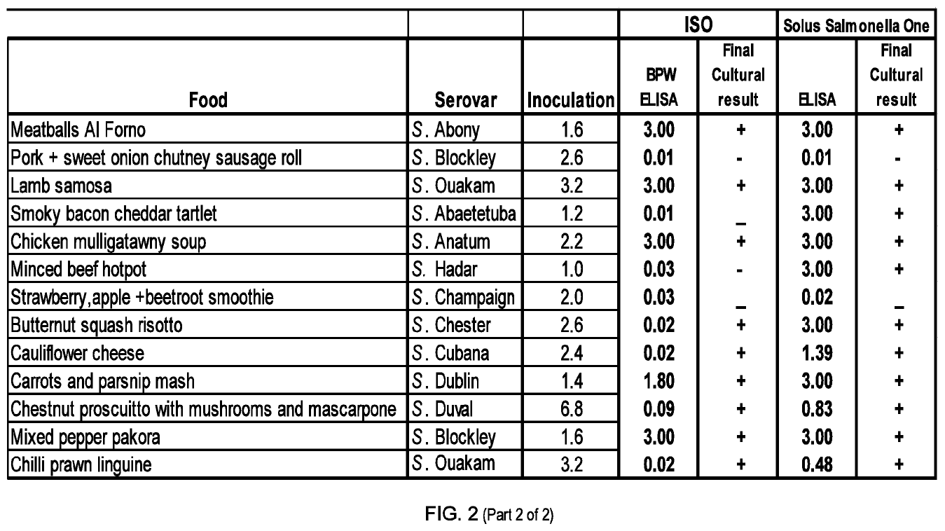

The inventors surprisingly found that the standard prior art methods of culturing Salmonella, are relatively slow and inadequate for current testing needs, (FIG. 2).

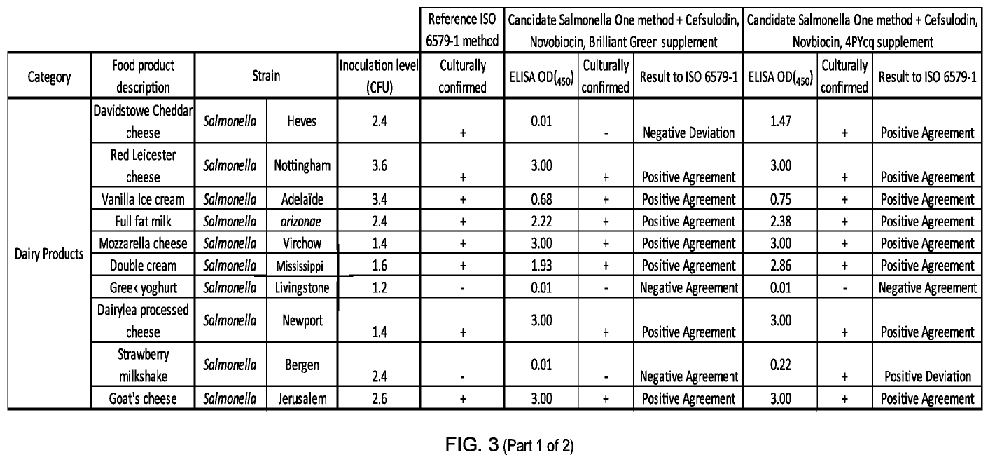

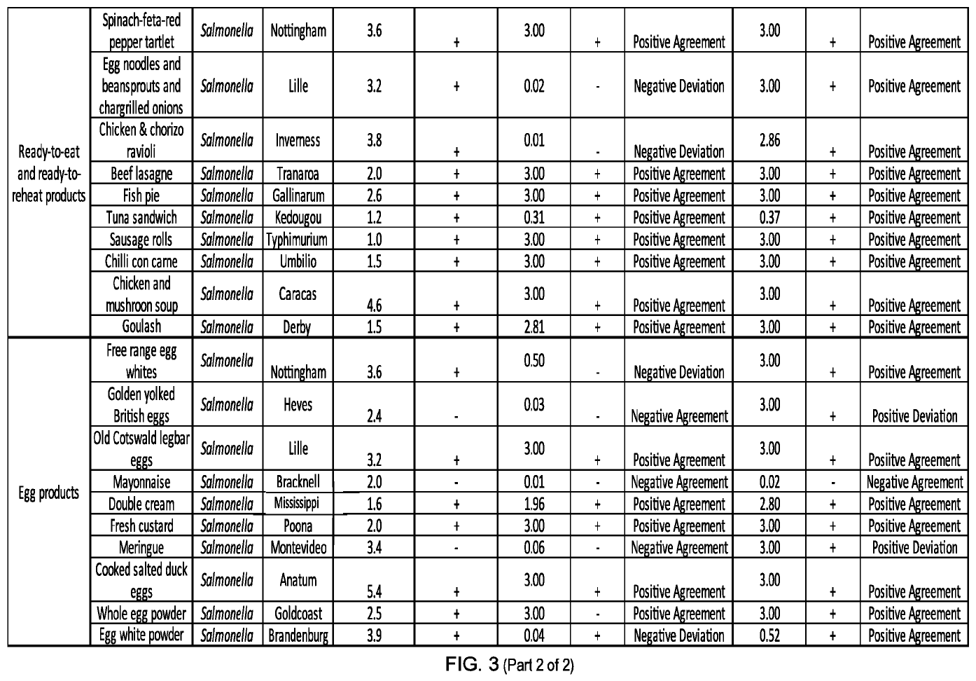

The strength of the detection signal obtained from cultures of various Salmonella strains cultured from various food backgrounds was much more variable when the cells were cultured using the standard current ISO method which is a 2-step culture compared to a method according to the disclosure which involves the use of an agent of formula I such as 4PYcq rather than BG, and a single culture step (FIG. 3). Reduced variability in the ability of a particular method to detect the presence of Salmonella, whatever the background contaminants (e.g. food debris) or Salmonella strain is a particular advantage of the present disclosure.

### Example 2—Culture with 4PYcg Allows Growth of Salmonella from Low Abundance Samples to Detectable Levels in 20 Hours Whereas Culture with BG does not

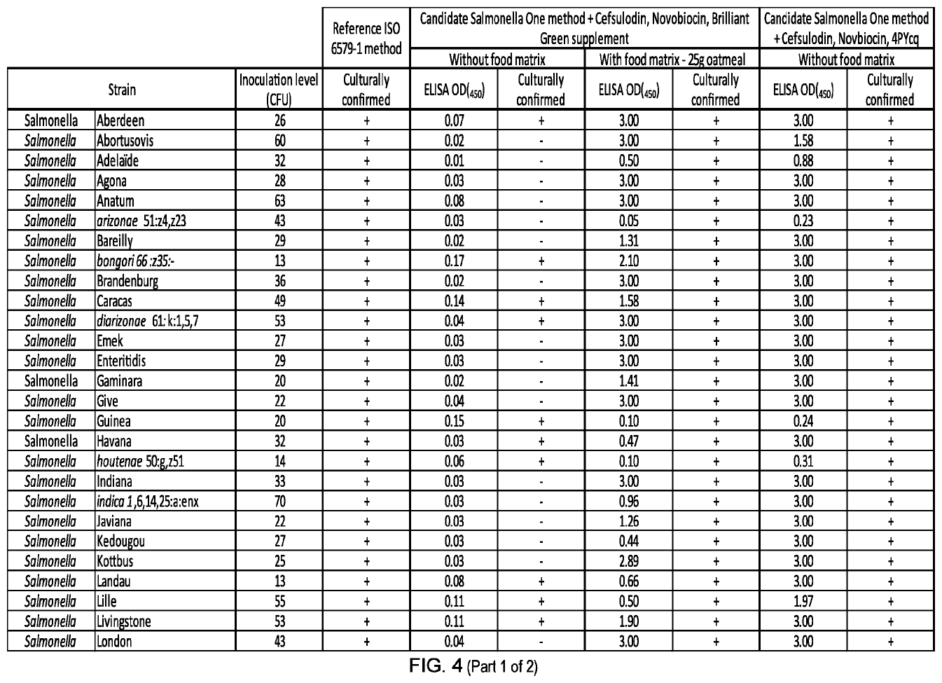

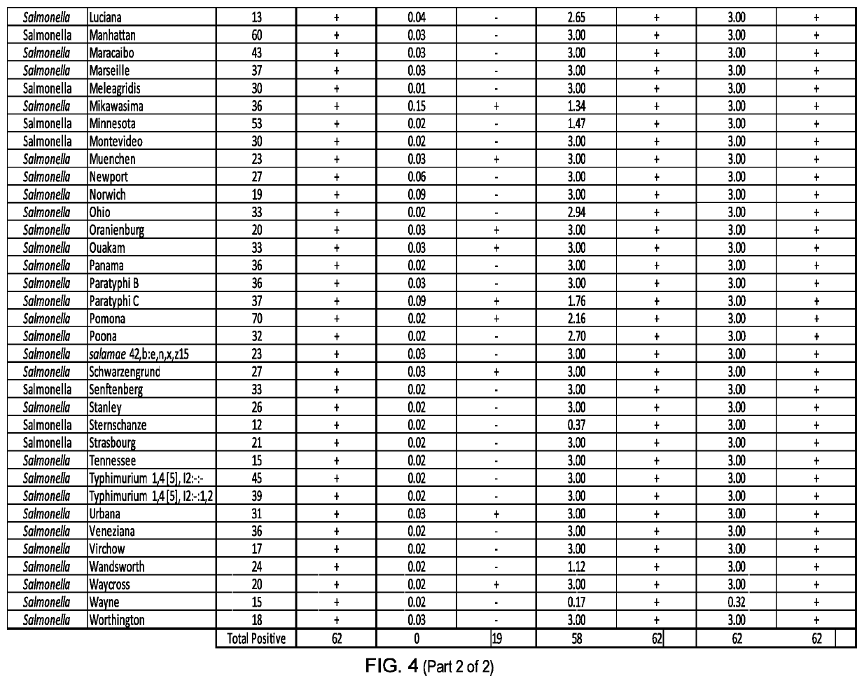

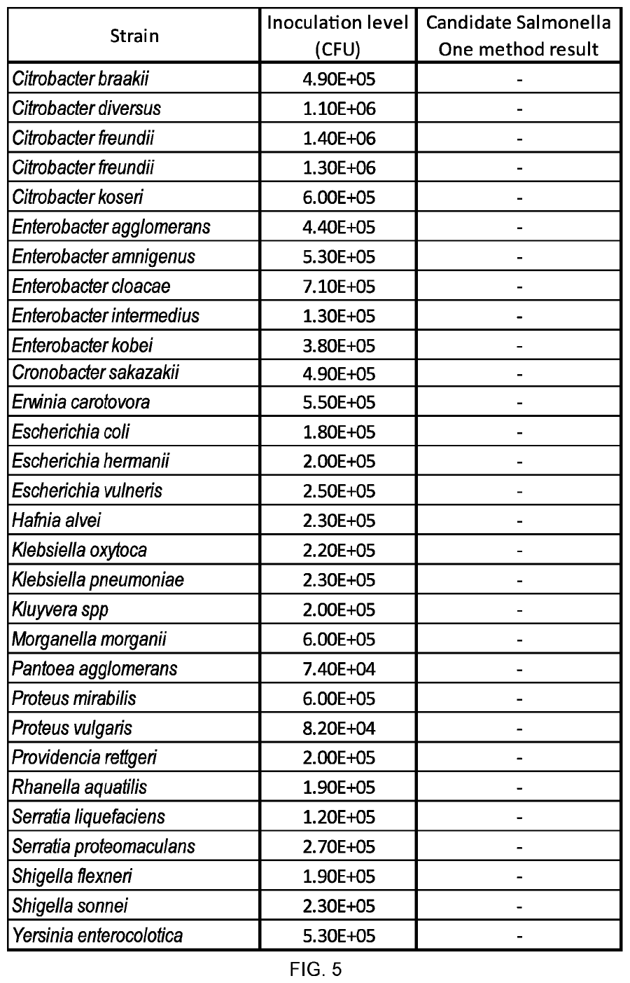

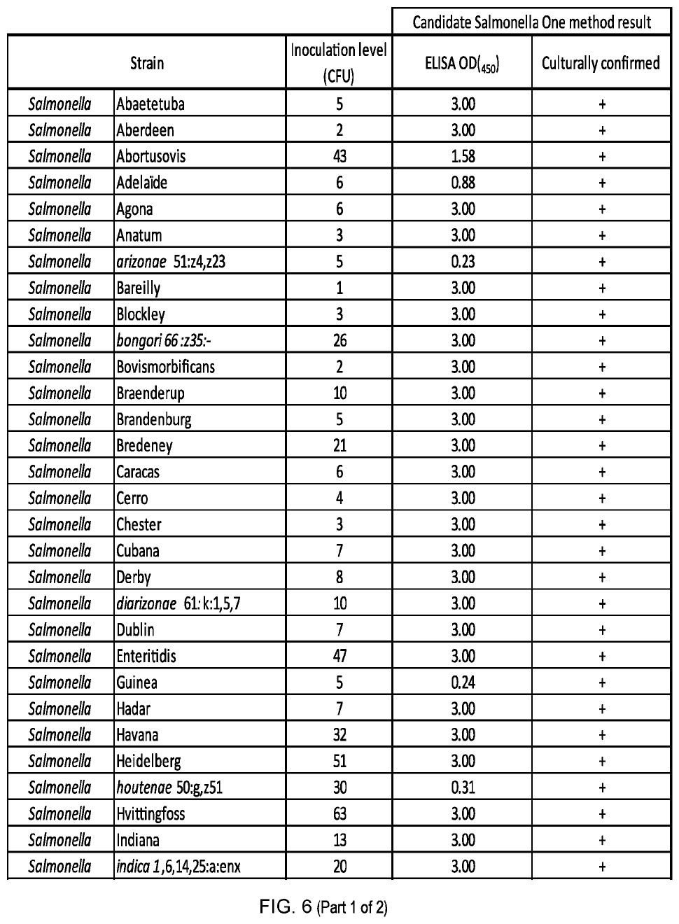

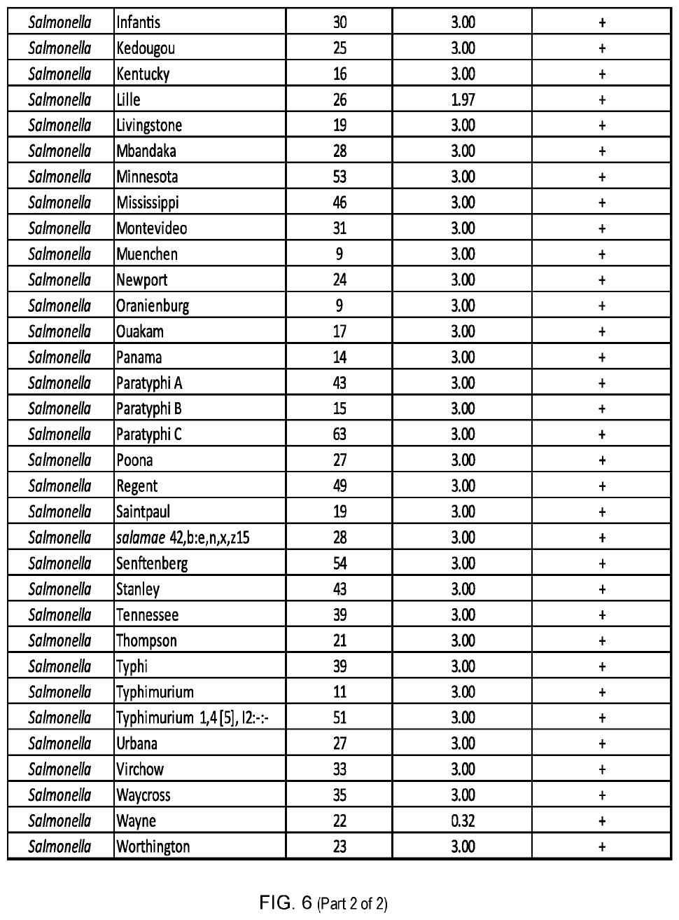

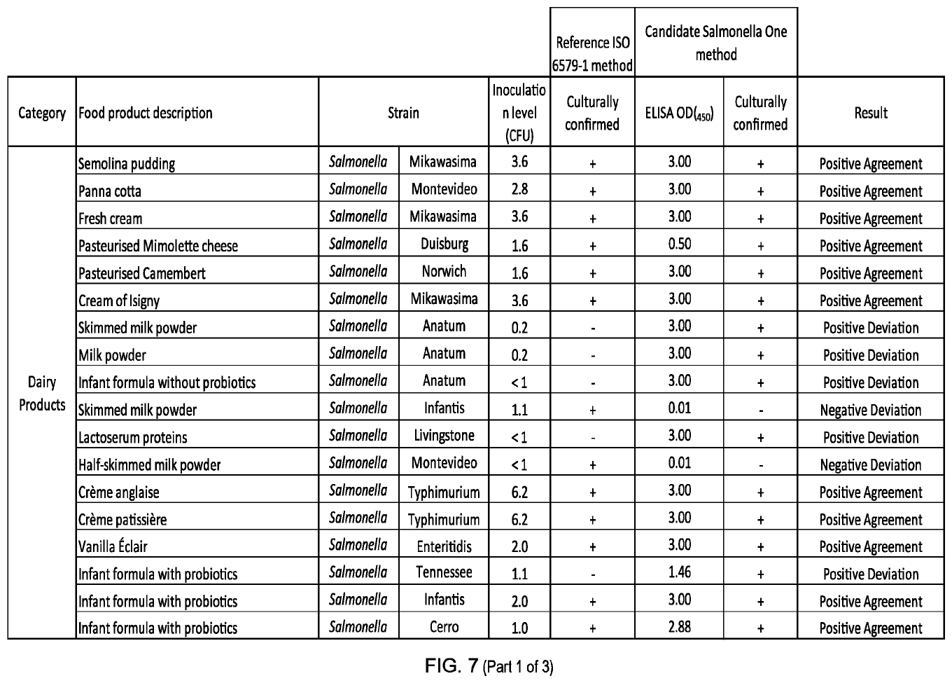

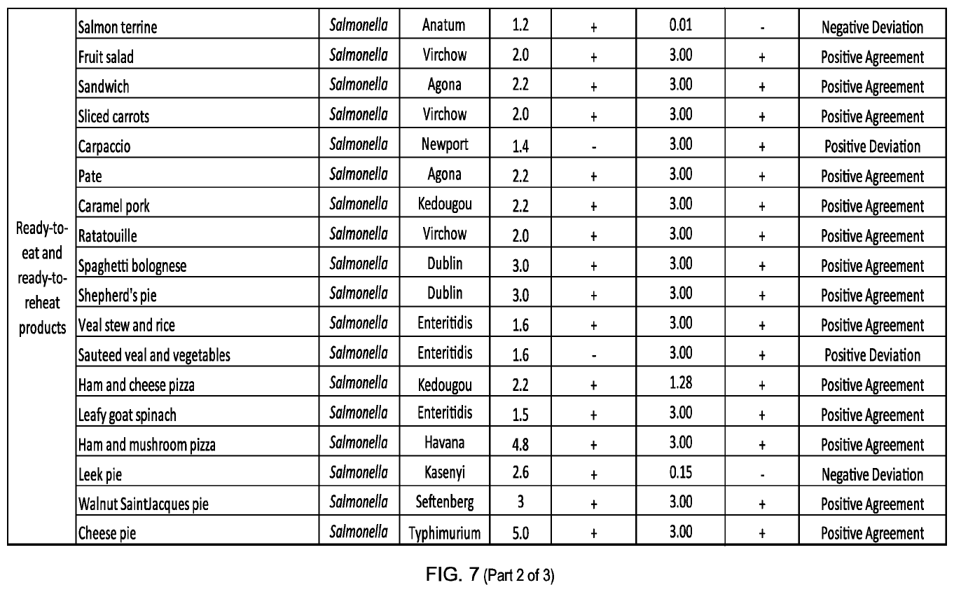

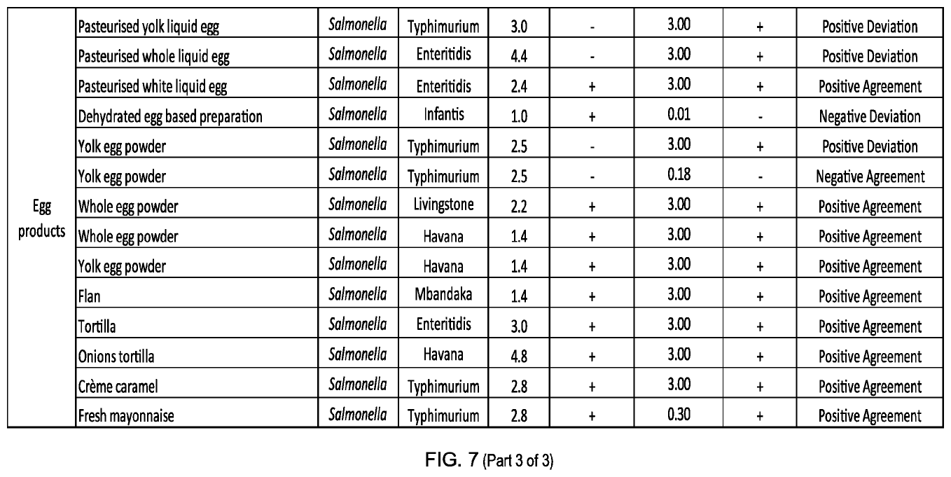

Furthermore, culture for 20 hours of low to moderate inoculum levels of pure Salmonella cultures with 8 mg/L BG resulted in insufficient recovery of Salmonella for detection using the ELISA method described herein (FIG. 4). However, 20 hour culture with 5 mg/L 4PYcq, the same concentration as used in the assays described herein and which results in sufficient suppression of competing microflora from a range of sample types, including for example dairy and probiotic foods of the same inoculums resulted in positive identification of the presence of Salmonella.

### Example 3—Reduced Salt Levels During Exposure of the Sample to the Detection Member Significantly Increases Sensitivity

TABLE 2
(signal)

| | Conjugate Diluent Type | | | | |
|---|---|---|---|---|---|
| | Hepes 25 mM pH 7.0 with 3D11-HRP @ 0.06 μg/ml | | | | |
| NaCl % | 0% | 0.2% | 0.4% | 0.6% | 0.8% |
| S. Enteritidis | 2.662 | 3.752 | 3.376 | 2.401 | 1.577 |
| 10⁸ cfu/ml | | | | | |
| S. Enteritidis | 1.626 | 2.389 | 1.083 | 0.599 | 0.308 |
| 10⁵ cfu/ml | | | | | |
| S. Typhimurium | 3.513 | 4.458 | 3.923 | 2.872 | 2.246 |
| 10⁶ cfu/ml | | | | | |
| S. Typhimurium | 1.016 | 1.978 | 0.940 | 0.531 | 0.324 |
| 10⁵ cfu/ml | | | | | |
| Blank value | 0.067 | 0.073 | 0.095 | 0.045 | 0.038 |

TABLE 3
(signal to noise ratio)

| | Conjugate Diluent Type | | | | |
|---|---|---|---|---|---|
| | Hepes 25 mM pH 7.0 with 3D11-HRP @ 0.06 μg/ml | | | | |
| NaCl % | 0% | 0.2% | 0.4% | 0.6% | 0.8% |
| S. Enteritidis | 40 | 52 | 36 | 53 | 42 |
| 10⁶ cfu/ml | | | | | |
| S. Enteritidis | 24 | 33 | 11 | 13 | 8 |
| 10⁵ cfu/ml | | | | | |
| S. Typhimurium | 52 | 61 | 42 | 63 | 59 |
| 10⁶ cfu/ml | | | | | |
| S. Typhimurium | 15 | 27 | 10 | 12 | 9 |
| 10⁵ cfu/ml | | | | | |
| Blank value | 0.067 | 0.073 | 0.095 | 0.045 | 0.038 |

Tables 2 and 3 show that an increase in sensitivity is seen when lower salt levels are present in the conjugate buffer. In particular, 0.2% NaCl (34.2 mM) conjugate buffer is optimal for detecting lower concentrations of S. Enteritidis and S. Typhimurium, such as at 1×10⁵ cfu/ml, ELISA plates used were coated directly with polyclonal anti-Salmonella antibody.

TABLE 4

| | Hepes 25 mM pH 7.0 | | | | | |
|---|---|---|---|---|---|---|
| | 3D11 HRP @ 0.04 μg/ml | | | | | |
| | % NaCl | | | | | |
| | | | | | | 10 mM |
| | 0% | 0.2% | 0.4% | 0.6% | 0.8% | dPBS |
| Serrratia liquefaciens | 0.070 | 0.102 | 0.089 | 0.052 | 0.046 | 0.040 |
| Shigella sonnei | 0.075 | 0.113 | 0.101 | 0.054 | 0.045 | 0.039 |
| Citrobacter freundii | 0.095 | 0.151 | 0.112 | 0.069 | 0.055 | 0.046 |
| Escherichia coli | 0.067 | 0.106 | 0.071 | 0.060 | 0.042 | 0.034 |
| Klebsiella pneumoniae | 0.065 | 0.109 | 0.067 | 0.061 | 0.040 | 0.035 |
| S. Enteritidis 10⁷ cfu/ml | 1.418 | 1.966 | 2.730 | 2.958 | 2.714 | 2.455 |
| S. Enteritidis 10⁶ cfu/ml | 2.657 | 3.762 | 3.041 | 2.345 | 1.424 | 1.155 |
| S. Enteritidis 10⁵ cfu/ml | 1.514 | 2.374 | 1.170 | 0.555 | 0.307 | 0.253 |
| Blank value | 0.066 | 0.124 | 0.085 | 0.073 | 0.042 | 0.036 |

Table 4 shows that lower levels of salt, for example, 0.2% NaCl (34.2 mM), intensifies the detection signal significantly for various strains of Salmonella. In this instance background signals increased with the fall in salt levels and there was evidence of a hook type effect.

### Example 4—Solus One Salmonella Kit Instructions

An exemplary method is detailed below.

This method is for the next day detection of Salmonella in food. This method is adapted to the detection of all Salmonella both motile and non-motile.

1. Introduction

Solus One Salmonella provides a negative or a presumptive positive result from a single enrichment step within 22 hours, of which the immunoassay is 2 hours

2. Intended Use

The test method requires laboratory facilities plus qualified and trained personnel. Basic training is recommended to first time users and can be provided by Solus Scientific Solutions Ltd. Using the method includes compliance with Good Laboratory Practices (refer to EN ISO 7218).

3. Reagents Provided

Kit components are supplied stabilised and ready to use at working concentration. Only the Washing Buffer Activator and Washing Buffer reagent require dilution. The activator must be first dissolved in 1440 ml of deionised (DI) water followed by the addition of the 60 ml concentrated Washing Buffer reagent to this solution.

Each kit contains sufficient material for 5×93 determinations, plus controls.

The kit expiry date is displayed on each product label.
5×96 well microplates (in breakable strip format). Wells are coated with antibodies against Salmonella spp.
Negative Control (Green label). 10 ml in working dilution. Contains diluent with preservative.
Positive Control (Red label). 10 ml in working dilution. Contains heat-killed Salmonella in diluent with preservative.
Conjugate (Orange label). 60 ml in working dilution. Contains horseradish peroxidase-antibody conjugate in diluent with preservative.
Substrate (Blue label). 60 ml in working dilution. Contains 3,3′5,5′-Tetramethylbenzidine (TMB), hydrogen peroxide and stabilisers. Solution should be clear or slightly faint blue.
Stop Solution (Yellow label). 60 ml in working dilution. Contains 10% sulphuric acid.
Washing Buffer Concentrate (25×). 5×60 ml.
Washing Buffer Activator. 5×1 Sachet

4. Materials and Equipment

Required but not Provided
Refrigerator at 2-8° C.
Deionised or distilled water
Buffered Peptone Water (BPWV) according to ISO 6579
SALSUPPII—Solus One Salmonella Supplement
70% v:v Ethanol
Measuring cylinder for 250 ml or 1 L
Filter bags (e.g. Stomacher)
Sterile 10 ml test tubes suitable for selective enrichment culture
Stomacher (or similar apparatus) and bags
3 ml transfer pipettes (sterile)
Test tube for sample boiling (e.g. 5 ml Poly propylene rimless test tubes 12×75 mm)
Vortex mixer
Timer
Incubator or water bath at 41.5±1° C.
Heating block or water bath (capable of heating to 85-100° C.)
Pipettes and tips (1 ml; 0.1 ml)
Microplate washer or squeeze bottle
Microplate reader with 450 nm filter
Autoclave for decontamination of samples

5. Reagent Preparation

5.1. Washing Buffer:

Prepare the following in a clean 2 litre vessel—

5.1.1 Add the contents of a Washing Buffer Activator Sachet to 1440 ml DI or distilled water and mix until the activator has fully dissolved.

5.1.2 Add 60 ml of the concentrated washing buffer reagent to the vessel containing 1440 ml of the dissolved activator solution.

5.1.3 Transfer to a storage bottle and label the solution as appropriate.

5.2. Culture Broth (growth medium): Prepare Buffered Peptone Water (BPW) ISO6579 following manufacturer's instructions. Allow to cool to room temperature before use in testing.

5.3. Prepare the Solus One Salmonella Supplement by the addition of 15 ml 70% v:v Ethanol. Allow the contents to dissolve into solution, this typically takes around 30 minutes at room temperature. Add 4.44 ml supplement per 1 L (1 ml per 225 ml) of BPW. (In some embodiments, half-strength supplement can be used, for example in some circumstances where the sample is a spice or a spicey product, or where the target cells are E. coli for example E. coli O157. In these situations, use 2.22 ml supplement per 1 L BPW).

6. Sample Preparation and Enrichment—Standard Method

Homogenise 25 g of the sample in 225 ml of supplemented BPW, and incubate for 20 to 22 hours at 41.5±1° C.

In the context of NF VALIDATION test portions weighing more than 25 g have not been tested. Refer to EN ISO 6579 for the specific preparations of the mother suspension for some foods.

Ensure that the bench processing time of supplemented BPW inoculated samples is kept to a minimum and transferred to the 41.5° C. incubator as soon as possible. This is important to avoid extensive growth of competing organisms.

When the incubation period in supplemented BPW is completed, carefully remove 1 ml, avoiding particulate and fatty matter (the use of a filter bag for this purpose is highly recommended), to a glass or polypropylene test tube.

Heat the aliquot to 85-100° C. for 15-20 minutes in the test tube. After heating allow the sample to cool to room temperature. This may be accelerated by placing the test tubes in cold water for 5 minutes.

Post boiling some samples could contain coagulated protein and fat content which may cause pipetting issues on the Dynex instrument. To avoid instrument errors add a frit to the boiling tube and gently push the frit beneath the surface of the liquid.

The un-boiled supplemented BPW samples should be kept for verification until immunoassay results are obtained. These samples can be kept at 41.5±1° C. if the immunoassay test is to be carried out within 2 hours or at 2-8° C. for up to 72 hours prior to the immunoassay test.

7. Immunoassay Procedure

7.1. Take the test kit from storage at 2-8° C. one hour before use to allow the components to reach room temperature. Determine the number of wells required for the test. Take the required number of strips from the pouch and fit them to the frame provided. Unused strips should be returned to the pouch and stored at 2-8° C.

7.2. Prepare Washing Buffer as detailed in section 5.1.

7.3. Leave the first well in the strip empty to serve as a ‘blank’ for measuring the absorbance of the substrate.

7.4. Pipette 0.1 ml of Negative Control (Green label) into the second well.

7.5. Pipette 0.1 ml of Positive Control (Red label) into the third well.

7.6. Pipette 0.1 ml of each boiled/cooled sample separately into consecutive wells in the strip. If there are wells left over at the end of a test strip the Positive or Negative Controls may be repeated.

7.7. Incubate the plate (containing the strips) at 37±1° C. for 30 mins (±5 mins.).

7.8. After incubation, aspirate the contents of the wells, removing as much of the liquid as possible. Wash the wells 5-7 times with Washing buffer ensuring complete filling and emptying of the wells through each wash cycle. The washing technique is critical to assay performance, hence it is recommended to use a microplate washer.

7.9. Pipette 0.1 ml of Conjugate (Orange label) into all wells except the ‘blank’.

7.10. Incubate the plate at 37±1° C. for 30 mins (±5 mins).

7.11. Repeat the wash cycles as detailed in section 7.9.

7.12. Pipette 0.1 ml of TMB Substrate (Blue label) into all wells, including the ‘blank’ well.

7.13. Incubate the plate at room temperature for 30 mins. (±5 mins).

7.14. After incubation stop the reaction by adding 0.1 ml of Stop Solution (Yellow label) to all wells including the ‘blank’ well. The Stop Solution will cause the blue colour in the wells to change to yellow.

7.15. Read the optical densities within 10 minutes in a plate reader using a 450 nm filter. Inspect the wells before reading for air bubbles and if present burst with a needle. Zero the reader against the ‘blank’ well before the other wells are read.

7.16. Do not use reference filter.

8. Interpretation of Results

Results are expressed as optical density (OD450) measurements using microplate reader. Subtract the OD value of the blank well (usually A1) from all of the other results.

Assay Acceptance Criteria:

Negative Control <0.100

OD450

Positive Control >0.500

OD450

Samples with OD450 readings of less than 0.200 are considered negative in which case the analysis is complete, the results may be reported and the corresponding non-boiled aliquot of supplemented BPW broth may be discarded following local regulations/guidelines.

Sample wells with OD450≥0.200 are considered presumptive positive for Salmonella. Presumptive positive results must be verified using a recognised culture method.

9. Confirmation of Positive Results from Salmonella Immunoassay

Samples with OD's≥0.200 are considered positive for Salmonella.

All samples identified as positive by the alternative method must be confirmed in one of the following ways.

The confirmation step must start from the (un-boiled) supplemented BPW samples stored at 41.5° C. or 2-8° C.:

Streak the supplemented BPW sample onto 1 agar plate (XLD or a chromogenic agar for Salmonella such as Colorex Salmonella from Chromagar). Incubate agar as specified by standard Salmonella cultural protocols and then perform confirmation tests e.g. Microgen latex test F42 or biochemical identification gallery directly on isolated colonies without purification step or by performing the tests described in the standardised methods (CEN or ISO). The F42 latex test uses polyclonal antibody to detect flagellar antigens. It is not adapted for the detection of non-motile Salmonella.

Or additionally subculture the supplemented BPW sample (0.1 ml+10 ml) in RVS broth incubated for 24 h+/−3 h at 41.5° c.+/−1° C. Streaking onto XLD or a chromogenic agar for Salmonella such as Colorex Salmonella from Chromagar). Incubate agar as specified by standard Salmonella cultural protocols and then perform confirmation tests e.g. Microgen latex test F42 or biochemical identification gallery directly on isolated colonies without purification step or by performing the tests described in the standardised methods (CEN or ISO).

In the event of discordant results (presumptive positive ELISA results, not confirmed by one of the means described above and in particular the latex test) the laboratory must follow the necessary steps to ensure the validity of the result obtained.

### Example 5 Effect of Salt in the Wash Solution on the Detection of False Positives

The following components/steps may be used but other components/steps are also possible.

Materials

Culture Media:

Streptavidin Plates:

The wells of a 94 well streptavidin plate are coated in 2 μg/ml biotinylated capture antibody.

Conjugate Buffer:

0.15 μg/ml conjugated 3D11 HRP

0.06 μg/ml of HRP conjugated antibody with binding affinity for other Salmonella components

25 mM HEPES

34.22 mM NaCl

BSA 1%

0.1% w/v Proclin 950

Potassium Ferricyanide 0.001%

Bovine gamma globulin 0.1%

It may also be suitable to supplement the conjugate buffer with detergent.

High Salt Wash Buffer:

4.073 mM Tween® 20

599 mM NaCl

Tris

Substrate:

This can be purchased as a proprietary solution: for example, Interchim TMB substrate product

code UP664782.

Stop Solution:

0.1M H₂SO₄

Method

The test sample was cultured in a culture medium comprising 5 mg/L 4PYcq and 10 mg/L novobiocin and 8 mg/L cefsulodin. The culture medium was incubated at 41.5° C. for 20 hours. An aliquot of the test sample was heated to 85-100° C. for 15-20 minutes, and allowed to cool to room temperature prior to the assay. 100 μl of the aliquoted test sample was added to the sample wells. The samples were incubated for 30 mins at 37±1° C. for 30 mins with mild agitation for the first 5 mins. All wells are washed 5-7 times with the high salt wash buffer. 100 μl of 0.15 μg/ml conjugated 3D11 HRP and 0.06 μg/ml of HRP conjugated antibody with binding affinity for other Salmonella components is added to the sample well and also the positive and negative control wells and incubated at 37±1° C. for 30 mins. The wells are washed 5-7 times with the high salt wash buffer. 100 μl substrate was then added to the well and incubated at 37±1° C. for 30 mins, after which 100 μl stop solution is then added. Optical density (OD) was recorded from the sample wells and also the positive and negative controls using a plate reader using a 450 nm filter within 10 minutes of the stop solution being added, and calibrated against the blank well.

Samples with OD450 readings of less than 0.200 are considered negative in which case the analysis is complete, the results may be reported and the corresponding non-boiled aliquot of supplemented BPW broth may be discarded.

Assay Acceptance Criteria:

| | |
|---|---|
| Negative Control OD450 | <0.100 |
| Positive Control OD450 | >0.500 |

Results

| | Streptavidin | Streptavidin | |
|---|---|---|---|
| | Biotin anti-Salmonella | | |
| | coating Antibody concentration | | |
| | 2 μg/ml | 2 μg/ml | |
| | Anti-Salmonella | | |
| | 3D11 HRP Concentration | | |
| | 0.15 μg/ml | 0.15 μg/ml | Final Culture |
| | Wash buffer salt levels | | Result for |
| | 0.8 | 3.5% | Presence of |
| | (137 mM) | (599 mM) | Salmonella |
| Gaucho sirloin steak | 0.010 | 0.010 | − |
| Wagu rump steak | 0.020 | 0.022 | − |
| Lean Belgian blue beef | 0.007 | 0.007 | − |
| rump steak | | | |
| Lean Italian buffalo | 0.011 | 0.010 | − |
| rump steak | | | |
| Wild boar steak | 0.064 | 0.031 | − |
| Venison haunch steaks | 0.013 | 0.010 | − |
| Diced mixed game | 0.255 | 0.060 | − |
| Buffalo quarter pounders | 0.015 | 0.009 | − |
| Picanha rump joint | 0.016 | 0.007 | − |
| Wild diced venison | 0.110 | 0.021 | − |
| Turkey thigh mince | 0.523 | 0.057 | − |
| Ostrich fillets | 0.009 | 0.006 | − |
| Whole stuffed partridge | 0.268 | 0.051 | − |
| Wood pigeon breasts | 0.047 | 0.022 | − |
| Chicken livers | 0.491 | 0.097 | − |
| Diced pheasant breast | 0.100 | 0.050 | − |
| Pheasant thighs | 0.195 | 0.039 | − |
| Turkey sausages | 3.000 | 2.664 | + |
| Diced turkey breast | 1.240 | 0.105 | − |
| Chicken wings | 0.087 | 0.032 | − |
| Chicken drumstick | 0.489 | 0.090 | − |
| Chicken thigh fillets | 1.429 | 0.186 | − |
| Chicken thighs | 0.030 | 0.007 | − |
| Chicken breast fillet | 0.033 | 0.013 | − |

Table 5 shows that the presence of supplementary NaCl in the wash buffer reduced the signals of false positives compared to wash buffer when NaCl was added at physiological levels (0.8%).

Effect of MgCl₂ in the Wash Buffer in Reducing the Frequency of False Positives

TABLE 6

| | Streptavidin | Streptavidin | Streptavidin |
|---|---|---|---|
| | Coating concentration of biotin-anti-Salmonella Ab | | |
| | 1.5 μg/ml | 1.5 ug/ml | 1.5 μg/ml |
| | Anti-Salmonella 3D11 HRP Concentration | | |
| | 0.1 μg/ml | 0.1 μg/ml | 0.1 μg/ml |
| | Wash buffer salt levels | | |
| | | 0.8% NaCl | 0.8% NaCl |
| | | (136 mM) + | (136 mM) + |
| | 0.8% | 0.125% | 0.0625% |
| | (136 mM) | (13 mM) | (6.6 mM) |
| | NaCl | MgCl₂ | MgCl₂ |
| False +ve poultry | 0.26 | 0.04 | 0.07 |
| sample | | | |
| False +ve poultry | 0.56 | 0.08 | 0.16 |
| sample | | | |
| False +ve poultry | 0.27 | 0.07 | 0.08 |
| sample | | | |
| False +ve poultry | 0.89 | 0.07 | 0.14 |
| sample | | | |
| False +ve poultry | 3.00 | 0.39 | 0.57 |
| sample | | | |
| False +ve poultry | 0.29 | 0.27 | 0.30 |
| sample | | | |
| False +ve poultry | 0.21 | 0.04 | 0.07 |
| sample | | | |
| False +ve poultry | 0.46 | 0.10 | 0.15 |
| sample | | | |
| False +ve poultry | 0.14 | 0.03 | 0.05 |
| sample | | | |
| False +ve poultry | 0.28 | 0.05 | 0.10 |
| sample | | | |
| S. Ealing | 1.10 | 0.65 | 0.72 |
| S. Adelaide | 1.30 | 0.74 | 0.80 |
| S. arizonae | 2.74 | 2.09 | 2.91 |
| S. Bracknell | 1.19 | 0.68 | 0.82 |
| S. Tranaroa | 3.00 | 3.00 | 3.00 |
| S. Alachua | 0.91 | 0.63 | 0.68 |
| S. Poona | 1.05 | 0.43 | 0.71 |
| S. Mississippi | 1.44 | 0.64 | 1.24 |
| S. Redeny | 3.00 | 3.00 | 3.00 |
| S. Caracas | 1.52 | 0.93 | 1.90 |
| S. Nottingham | 1.91 | 0.76 | 1.39 |
| 10⁶ cfu/ml | | | |
| S. Nottingham | 0.17 | 0.08 | 0.13 |
| 10⁵ cfu/ml | | | |
| S. Enteritidis | 3.00 | 2.95 | 3.00 |
| 10⁶ cfu/ml | | | |
| S. Enteritidis | 2.09 | 0.32 | 0.61 |
| 10⁵ cfu/ml | | | |
| S. Typhimurium | 3.00 | 2.03 | 3.00 |
| 10⁶ cfu/ml | | | |
| S. Typhimurium | 1.11 | 0.27 | 0.48 |
| 10⁵ cfu/ml | | | |
| Negative control | 0.02 | 0.03 | 0.03 |
| Positive control | 1.72 | 1.15 | 1.32 |

Table 6 shows that MgCl₂ can be added to the wash buffer to reduce false positive signals although some signal loss for samples containing Salmonella was observed.

Effect of KCl in the Wash Buffer on Assay Specificity

| | Plate coated with anti-Salmonella | |
|---|---|---|
| | biotin-polyclonal Ab at 1.5 μg/ml | |
| | Streptavidin | Streptavidin |
| | 3D11 HRP Concentration | |
| | 0.10 μg/ml | 0.10 μg/ml |
| | 50 μl MgCl₂ Sample additive to 100 μl of sample | |
| | 3.75% | 3.75% |
| | (394 mM) | (394 mM) |
| | Wash buffer NaCl Concentration | |
| | 0.8% NaCl | 0.8% NaCl |
| | (137 mM) | (137 mM) |
| | Wash buffer KCl Concentrations | |
| | | 2.75% KCl |
| | 0% | (370 mM) |
| False positive poultry | 0.09 | 0.06 |
| sample | | |
| False positive poultry | 0.23 | 0.09 |
| sample | | |
| False positive poultry | 0.17 | 0.08 |
| sample | | |
| False positive poultry | 0.41 | 0.20 |
| sample | | |
| False positive poultry | 0.31 | 0.11 |
| sample | | |
| False positive poultry | 0.19 | 0.08 |
| sample | | |
| False positive poultry | 0.25 | 0.07 |
| sample | | |
| False positive poultry | 0.37 | 0.10 |
| sample | | |
| False positive poultry | 0.61 | 0.19 |
| sample | | |
| False positive poultry | 0.44 | 0.12 |
| sample | | |
| False positive poultry | 0.27 | 0.24 |
| sample | | |
| S. Enteritidis | 3.00 | 3.00 |
| 1 × 10⁷ cfu/ml | | |
| S. Enteritidis | 3.00 | 3.00 |
| 1 × 10⁸ cfu/ml | | |
| S. Enteritidis | 3.00 | 3.00 |
| 1 × 10⁵ cfu/ml | | |
| S. Enteritidis | 0.72 | 0.63 |
| 1 × 10⁴ cfu/ml | | |
| S. Typhimurium | 3.00 | 3.00 |
| 1 × 10⁷ cfu/ml | | |
| S. Typhimurium | 3.00 | 3.00 |
| 1 × 10⁶ cfu/ml | | |
| S. Typhimurium | 2.25 | 3.00 |
| 1 × 10⁵ cfu/ml | | |
| S. Typhimurium | 0.32 | 0.28 |
| 1 × 10⁴ cfu/ml | | |

Table 8 shows the effect of adding supplementary KCl to the wash buffer has a similar effect to NaCl on reducing false positive signals. In this assay 50 μl of the MgCl₂ sample additive as added to 100 μl of sample prior to incubation.

### Example 6—Preferred Use of Streptavidin Coated Assay Plates

TABLE 7

| | Passively | |
|---|---|---|
| | coated | Streptavidin |
| | Anti-Salmonella | |
| | Coating Antibody concentration | |
| | 8 ug/ml | 2 ug/ml |
| | Anti-Salmonella | |
| | 3D11 HRP Concentration | |
| | 0.1 ug/ml | 0.1 ug/ml |
| | 50 μl Sample Additive (+100 μl sample) | |
| | MgCl₂ 3.75% | MgCl₂ 3.75% |
| | (394 mM) | (394 mM) |
| | Wash Buffer salt levels | |
| | 3.5% NaCl | 3.5% NaCl |
| | (599 mM) | (599 mM) |
| False +ve poultry sample | 0.05 | 0.03 |
| False +ve poultry sample | 0.10 | 0.05 |
| False +ve poultry sample | 0.04 | 0.03 |
| False +ve poultry sample | 0.13 | 0.04 |
| False +ve poultry sample | 0.14 | 0.08 |
| False +ve poultry sample | 0.10 | 0.05 |
| False +ve poultry sample | 0.17 | 0.08 |
| False +ve poultry sample | 0.07 | 0.03 |
| False +ve poultry sample | 0.10 | 0.05 |
| False +ve poultry sample | 0.15 | 0.06 |
| False +ve poultry sample | 0.07 | 0.05 |
| False +ve poultry sample | 0.19 | 0.06 |
| False +ve poultry sample | 0.06 | 0.03 |
| False +ve poultry sample | 0.08 | 0.04 |
| False +ve poultry sample | 0.19 | 0.06 |
| False +ve poultry sample | 0.15 | 0.06 |
| False +ve poultry sample | 0.07 | 0.04 |
| Salmonella +ve poultry | 1.31 | 2.58 |
| sample | | |
| Salmonella +ve poultry | 0.21 | 0.41 |
| sample | | |
| Salmonella +ve poultry | 1.70 | 2.34 |
| sample | | |
| Salmonella +ve poultry | 3.00 | 3.00 |
| sample | | |
| Salmonella +ve poultry | 3.00 | 3.00 |
| sample | | |
| Salmonella +ve poultry | 1.78 | 3.00 |
| sample | | |
| Salmonella +ve poultry | 0.43 | 0.99 |
| sample | | |
| Salmonella +ve poultry | 1.12 | 2.43 |
| sample | | |
| Salmonella +ve poultry | 0.77 | 1.09 |
| sample | | |
| Salmonella +ve poultry | 0.55 | 1.04 |
| sample | | |
| Salmonella +ve poultry | 0.14 | 0.48 |
| sample | | |
| Salmonella +ve poultry | 0.09 | 0.12 |
| sample | | |
| Salmonella Enteritidis 10⁷ | 3.00 | 3.00 |
| cfu/ml | | |
| Salmonella Enteritidis 10⁶ | 3.00 | 3.00 |
| cfu/ml | | |
| Salmonella Enteritidis 10⁵ | 0.85 | 1.20 |
| cfu/ml | | |
| Salmonella Typhimurium 10⁷ | 3.00 | 3.00 |
| cfu/ml | | |
| Salmonella Typhimurium 10⁶ | 3.00 | 3.00 |
| cfu/ml | | |
| Salmonella Typhimurium 10⁵ | 0.39 | 0.76 |
| cfu/ml | | |

Table 7 shows that using biotinylated polyclonal capture antibody bound to a streptavidin coated plate increases the signal of samples containing Salmonella and also improves assay specificity, compared to the same capture antibody passively bound the polystyrene plate.

### Example 7—Use of Alternative Monoclonal Antibody to Detect Salmonella

Plate coated with anti-Salmonella
biotin-polyclonal Ab at 1.5 μg/ml

| | |
|---|---|
| Streptavidin | Streptavidin |

Alternative Anti-Salmonella
LPS HRP Conjugate concentration

| | |
|---|---|
| 0.20 μg/ml | 0.20 μg/ml |

Wash buffer Salt Levels

| | 0.8% | 3.5% |
|---|---|---|
| | (137 mM) | (599 mM) |
| False +ve poultry sample | 0.30 | 0.09 |
| False +ve poultry sample | 0.49 | 0.10 |
| False +ve poultry sample | 0.24 | 0.10 |
| False +ve poultry sample | 0.26 | 0.09 |
| False +ve poultry sample | 0.98 | 0.08 |
| False +ve poultry sample | 0.38 | 0.10 |
| False +ve poultry sample | 1.29 | 0.10 |
| False +ve poultry sample | 0.72 | 0.12 |
| False +ve poultry sample | 0.19 | 0.11 |
| False +ve poultry sample | 0.17 | 0.09 |
| False +ve poultry sample | 0.33 | 0.10 |
| False +ve poultry sample | 1.90 | 0.08 |
| False +ve poultry sample | 0.29 | 0.08 |
| False +ve poultry sample | 0.83 | 0.10 |
| False +ve poultry sample | 0.28 | 0.09 |
| False +ve poultry sample | 0.85 | 0.09 |
| False +ve poultry sample | 1.20 | 0.10 |
| False +ve poultry sample | 0.43 | 0.09 |
| False +ve poultry sample | 0.27 | 0.09 |
| S. Enteritidis 10⁷ cfu/ml | 3.00 | 2.54 |
| S. Enteritidis 10⁶ cfu/ml | 3.00 | 2.71 |
| S. Enteritidis 10⁵ cfu/ml | 3.00 | 0.53 |
| S. Typhimurium 10⁷ cfu/ml | 3.00 | 3.00 |
| S. Typhimurium 10⁶ cfu/ml | 3.00 | 1.91 |
| S. Typhimurium 10⁵ cfu/ml | 2.62 | 0.32 |

Table 9 shows that a similar monoclonal to 3D11 can also be used in the present disclosure to detect Salmonella cells.

Selective Growth of Salmonella

ISO Buffered Peptone Water

| Formulation | g/L |
|---|---|
| Enzymatic digest of casein | 10 |
| Sodium chloride | 5.0 |
| Disodium hydrogen phosphate anhydrous | 3.6 |
| Potassium dihydrogen phosphate | 1.5 |

The above medium is made selective to prevent the overgrowth of the enrichment with competing flora allowing more unrestricted growth of Salmonella.

| Selective supplement | mg/L |
|---|---|
| 4PYcq | 5.0 |
| Novobiocin | 10.0 |
| Cefsulodin | 8 |

See FIG. 3

### Example 8—Recovery and Detection of Salmonella from Test Samples that Comprise Spices, Herbs and Flavourings

Microbes, such as Salmonella, are notoriously difficult to recover from samples of spices, flavourings and herbs due, in part, to the phenolic compounds that are present in spices, flavourings and herbs. Recovery is typically performed by diluting out the spices and flavourings from the sample. This requires a suitably high initial inoculum level of microbes so that sufficient microbial cells are present in the diluted sub-sample which is taken for recovery and testing. At least for this reason, current methods for recovering and detecting microbes such as Salmonella from spices, flavourings and herbs, are unreliable and inaccurate and are not suitable for routine high-throughput, rapid testing.

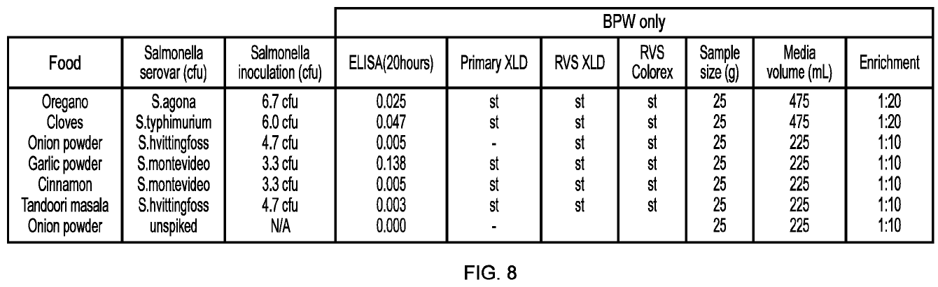

FIG. 8 shows that culturing Salmonella cells from various spices, flavourings and herbs in BPW media alone in a single step, as described herein, is difficult, if not possible.

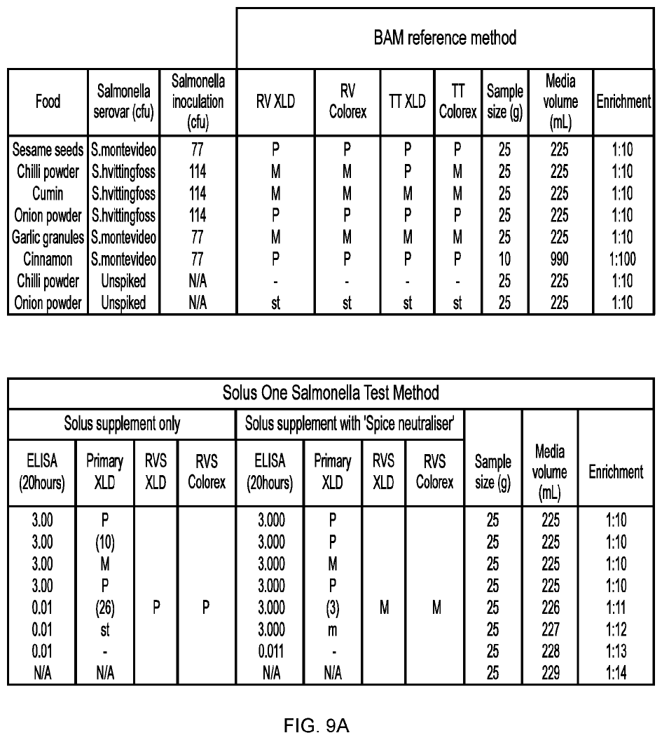

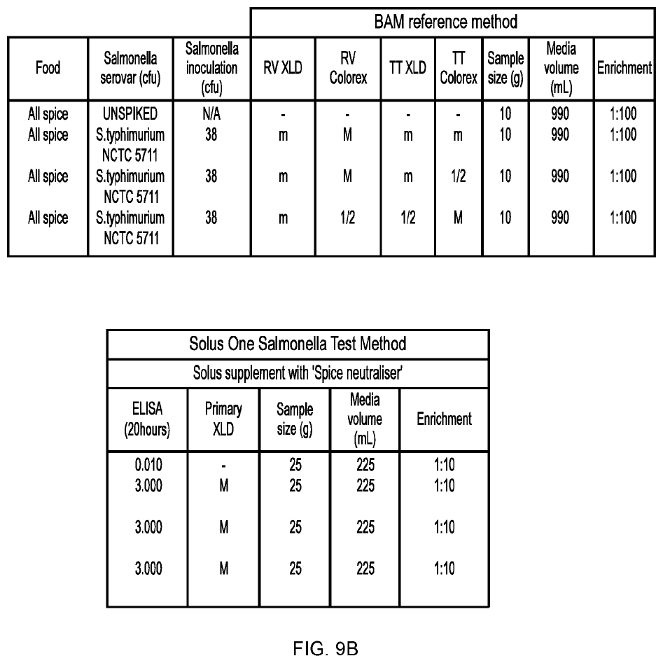

However, FIG. 9A shows that the inclusion of the selective supplement comprising 5 mg/L 4PYcq, 8 mg/L Cefsulodin and 10 mg/L Novobiocin as described herein, allows the recovery of Salmonella cells from a variety of different spices, herbs and flavourings. However, the recovery of Salmonella present in garlic granules and cinnamon in a single culture step was not sufficient to allow detection.

The inventors have surprisingly found that the inclusion of an additional 2 agents, ferric chloride and a sulphite compound such as potassium sulphite, in addition to the 4PYcq/Cefsulodin/Novobiocin supplement, not only allows recovery of Salmonella from these samples (which is achieved with the 4PYcq/Cefsulodin/Novobiocin supplement), but provides results which show equivalence with the results obtained from the BAM reference method (10-100 g/1:10-1:100 enrichment). See FIGS. 9A and 9B.

The data provided in FIGS. 9A and 9B are derived from samples that were inoculated with a relatively high level of Salmonella (FIG. 9A—77-114cfu; FIG. 9B—38 cfu).

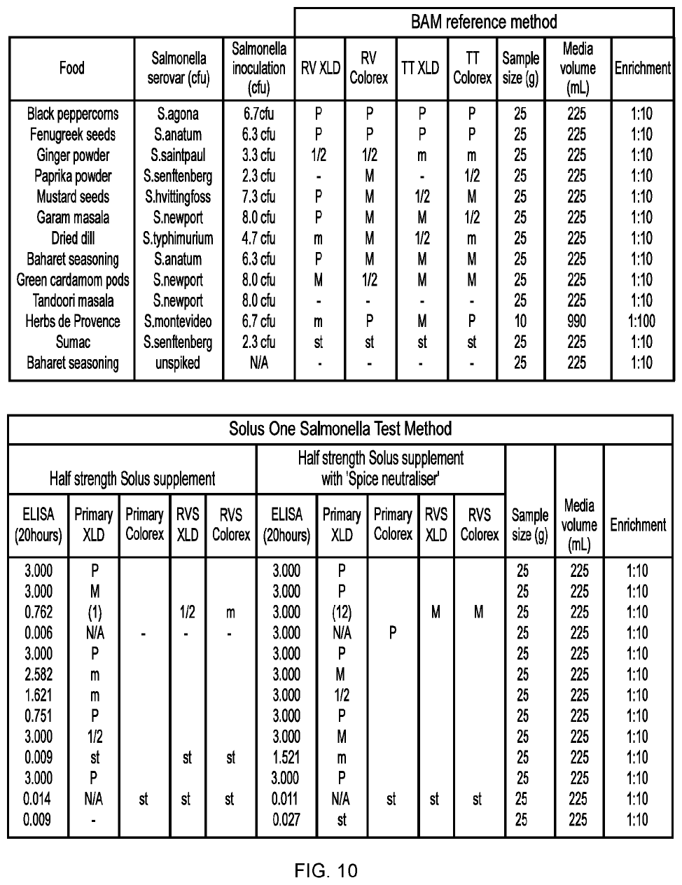

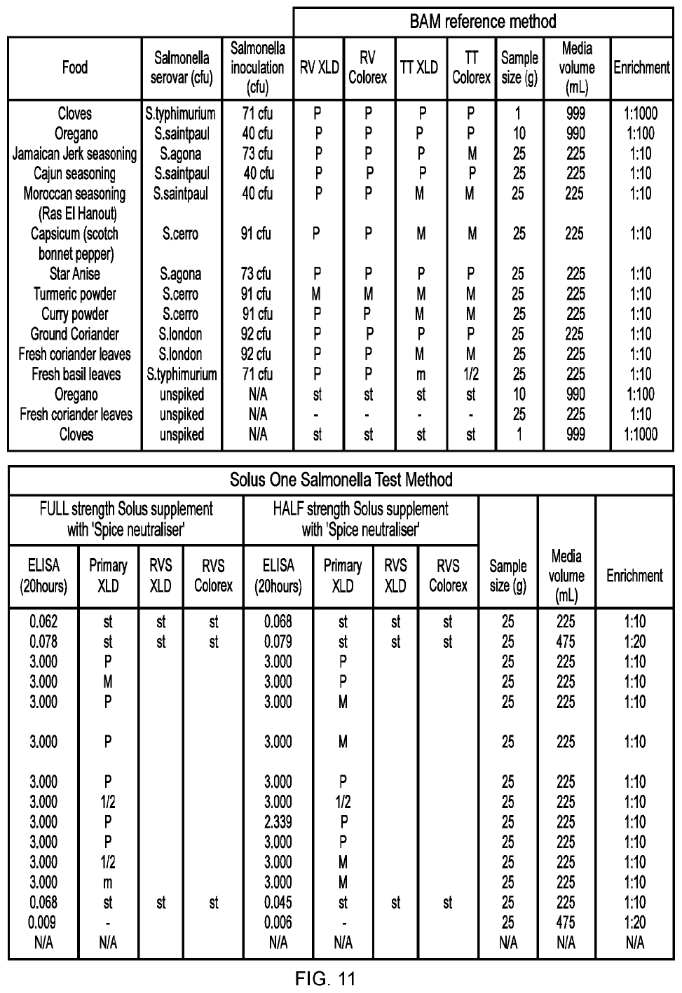

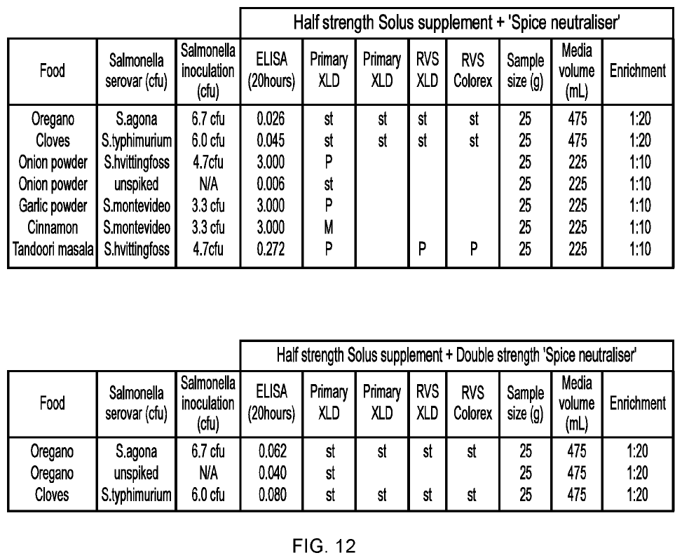

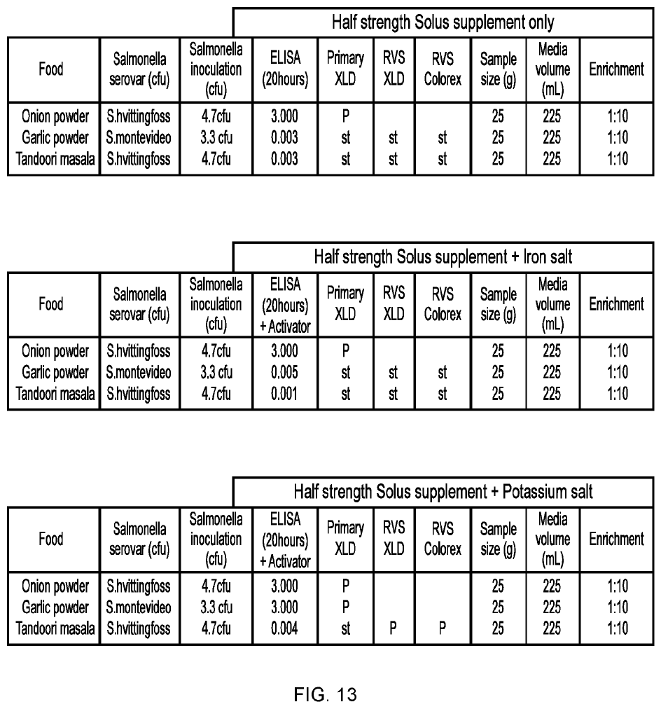

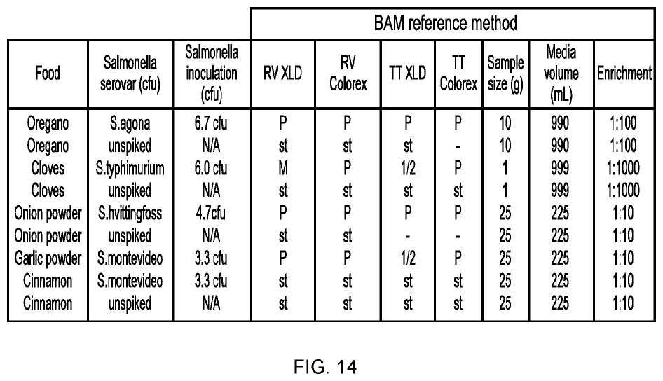

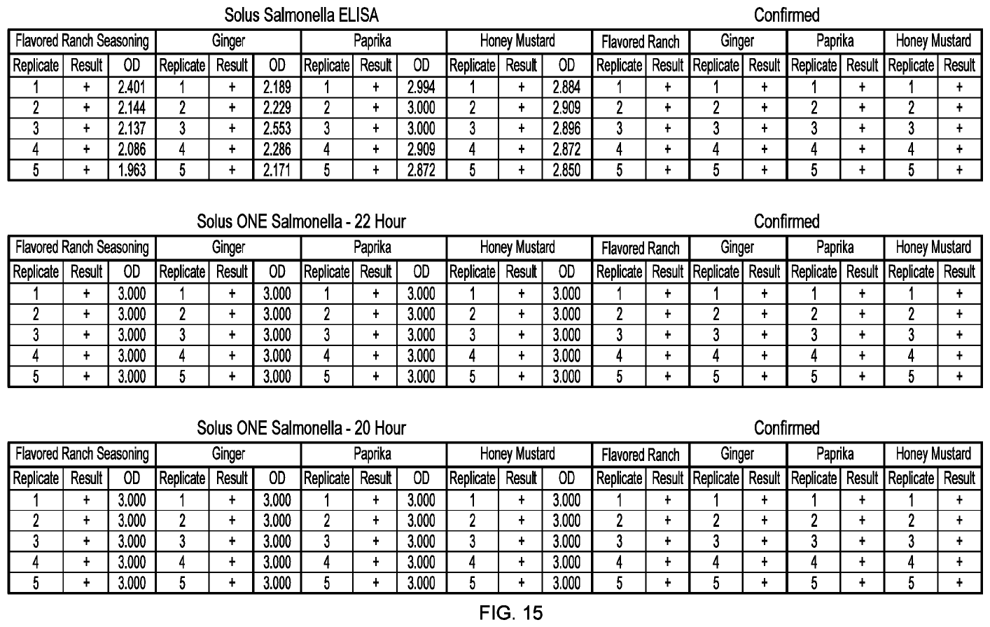

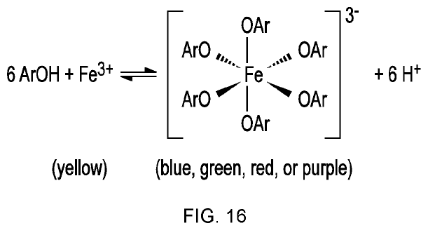

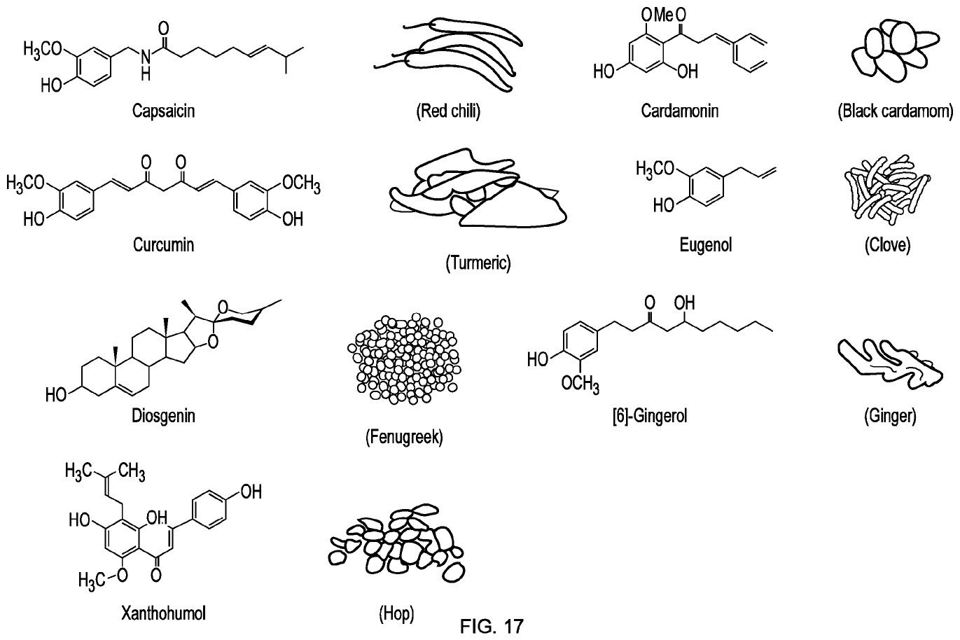

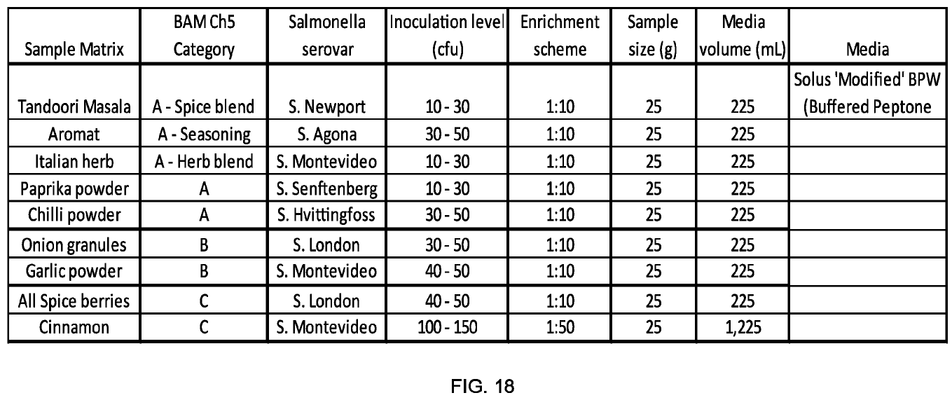

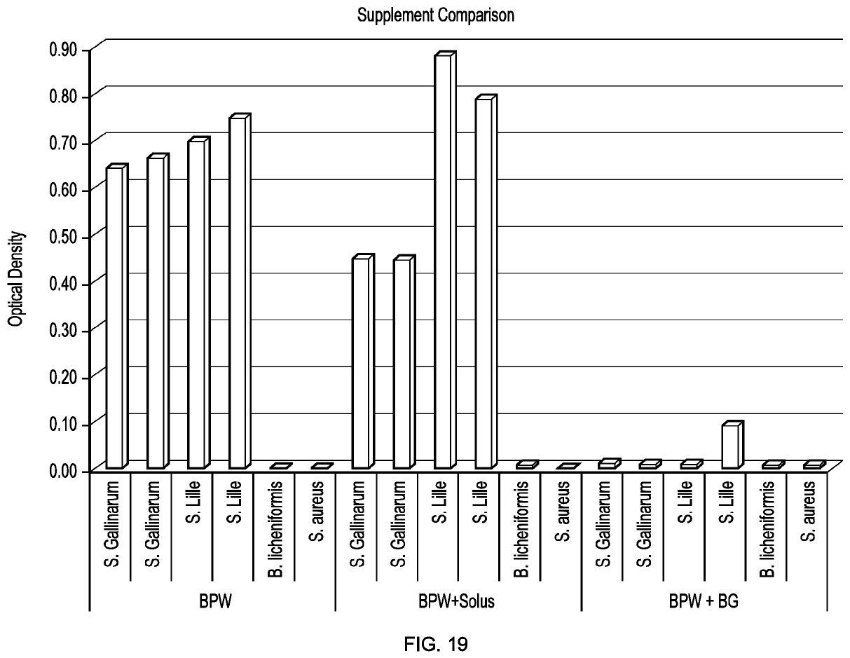

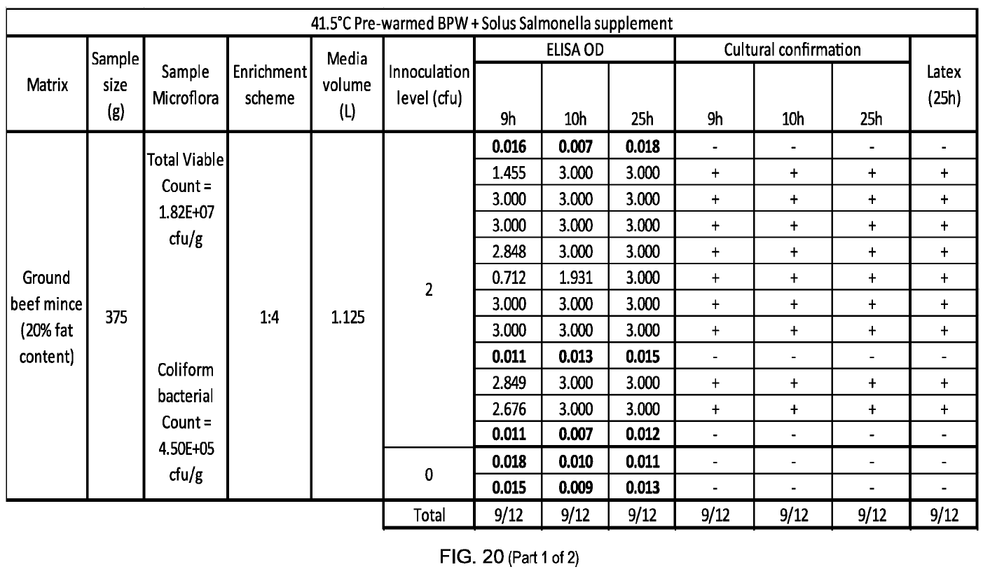

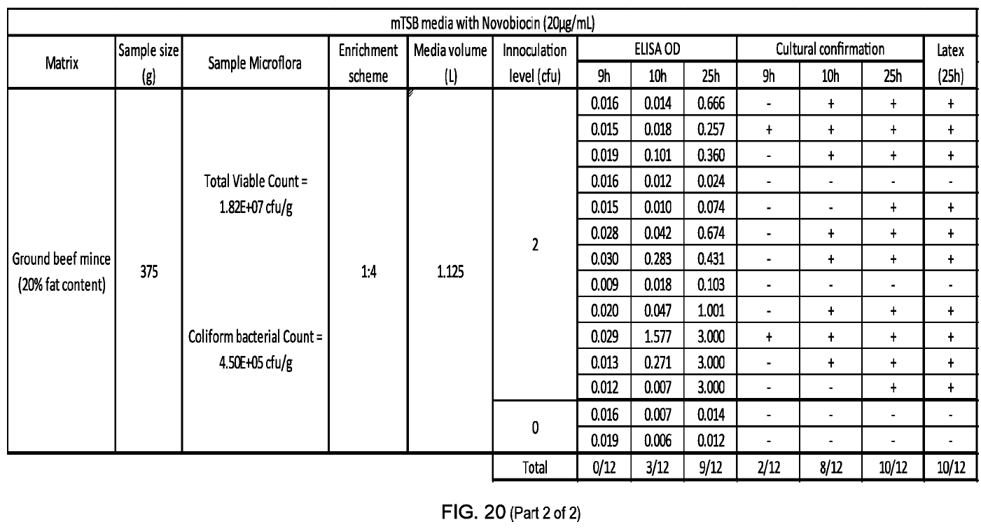

FIG. 10 provides data derived from fractional inoculation levels (2-8 cfu). In this instance the cells are cultured in the 4PYcq/Cefsulodin/Novobiocin supplement at half the concentration of that used previously, i.e. at a working concentration of 2.5 mg/L 4PYcq, 4 mg/L Cefsulodin and 5 mg/L Novobiocin. Both the standard concentration (5 mg/L 4PYcq, 8 mg/L Cefsulodin and 10 mg/L Novobiocin) and the half concentration (2.5 mg/L 4PYcq, 4 mg/L Cefsulodin and 5 mg/L Novobiocin) have been shown to be equivalent in their ability to recover Salmonella from spices, herbs and flavourings (see FIG. 11).

FIG. 10 shows that even at fractional inoculation levels, the inclusion of ferric chloride and potassium sulphite (the “spice neutraliser” supplement) allows the recovery and detection of Salmonella from spices, herbs and flavourings.

Recovery and detection of Salmonella from cloves and oregano is however more difficult, and a higher initial inoculation level may be required. This is true even when the “spice neutraliser” composition is used at double strength (FIG. 12).

FIG. 13 shows the relevance of each of the components of the “spice neutraliser” (ferric chloride and potassium sulphite).

With spices/flavourings from the allium family, the potassium salt component in Solus's ‘Spice neutraliser’ is sufficient for recovery of Salmonella.

For flavouring and pure products just containing onion, the presence of the Solus supplement is sufficient.

For different spices such as Tandoori masala, we require both the iron and potassium salts in the complete ‘spice neutraliser’ to get effective recovery of Salmonella. See for example FIG. 10 which shows that recovery from Tandoori masala is good when cultured in the presence of both agents of the “spice neutraliser”, i.e. in the presence of ferric chloride and potassium sulphite.

FIG. 15 shows validation data, comparing a standard 2-step culture protocol, with potassium sulphite and ferric chloride, to a single-culture method according to the disclosure, also comprising ferric chloride, potassium sulphite and the selective supplement comprising 4PYcq, Novobiocin and Cefsulodin.

FIG. 17 shows the structures of some anti-microbial components of some spices.

### Example 9—Methods and Compositions According to the Disclosure are Suitable for the Culture and Detection of Gram Negative Bacteria Other than Salmonella

Half-strength supplement in BPW was used to culture E. coli O157 from 375 g beef samples (ground mince & raw beef trim)/1:4 enrichment. Culture after only 8-10 hours shows equivalence to some of the molecular methods currently available. See FIG. 20.

1. A method for selectively culturing Salmonella or E. coli, the method comprising:
culturing microorganisms present in a test sample in a culture medium, wherein the culture medium comprises an agent of Formula I 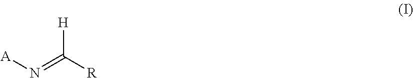
wherein A is selected from 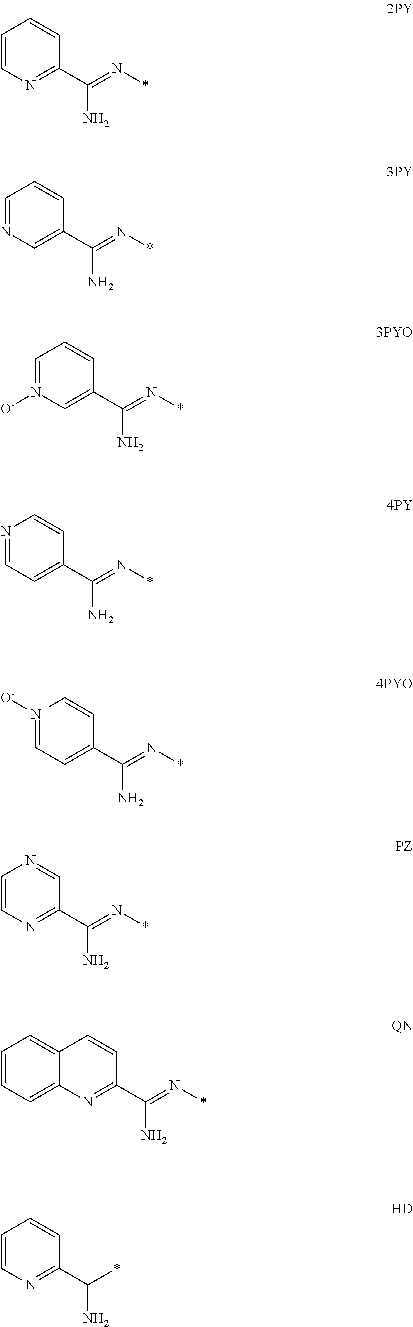
and R is selected from substituted C₅₋₂₀ aryl, with the proviso that when A is 2PY, then R is not 1,3-dimethylphenyl.

2. The method of claim 1 wherein the agent of Formula I is selected from the group consisting of 3PYaf, 4PYaf, 4PYam, 4PYcb, 4PYco, 4PYcq, 4PYeh, HDcb, HDce, HDcf and HDdb.

3. The method of claim 1, wherein the agent of Formula I is present in the culture media in a concentration of:
at least 0.5 mg/L; and
no more than 15 mg/L.

4. The method according to claim 1, wherein the E. coli is a Shiga toxin producing E. coli.

5. (canceled)

6. The method according to claim 1, wherein the culture medium further comprises an anti-bacterial agent, wherein the anti-bacterial agent comprises cefsulodin or novobiocin.

7. The method according to claim 1, wherein the culture medium further comprises at least one of:
cefsulodin at a concentration of from 1 mg/L to 20 mg/L;
novobiocin in an amount of from 2 mg/L to 40 mg/L;
ferric chloride at a concentration of from 0.25 mg/L to 4 mg/L;
a sulphite compound at a concentration of from 0.5 g/L to 4.0 g/L.

8. The method according to claim 1, wherein the test sample is a sample that comprises or is expected to comprise at least one or more cells other than a Salmonella or E. coli cell.

9. The method of claim 1, wherein the test sample comprises at least one of:
a food product;
a clinical sample;
a therapeutic product;
a swab;
a cosmetic.

10. The method according to claim 1, wherein the test sample is not a pure culture of Gram negative bacteria.

11. The method according to claim 1, wherein the method comprises a single culture step.

12. The method of claim 1, wherein the test sample is cultured in a culture medium for:
from 10 to 18 hours;
less than 24 hours; or
for 16 hours at a temperature from 30° C. to 44° C.

13. (canceled)

14. (canceled)

15. A culture medium for the selective growth of Salmonella cells or E. coli cells that may be present in a test sample, the culture medium comprising:
an agent of Formula I as defined in claim 1;
at least one of:
cefsulodin; or
novobiocin.

16. (canceled)

17. The culture medium according to claim 15, wherein the culture medium comprises at least one of:
cefsulodin at a concentration of from 1 mg/L to 20 mg/L; or
novobiocin in an amount of from 2 mg/L to 40 mg/L.

18. The culture medium according to claim 15, wherein the culture medium further comprises at least one of:
ferric chloride; or
a sulphite compound.

19. (canceled)

20. (canceled)

21. A composition comprising:
an agent of Formula I as defined in claim 1;
at least one of:
cefsulodin; or
novobiocin.

22. The composition according to claim 21 wherein the composition further comprises at least one of:
ferric chloride; or
a sulphite compound.

23. The composition according to claim 22, wherein the concentration of:
the agent of Formula I is at least 1.00 mg/ml and no more than 1.50 mg/ml;
novobiocin is at least 2.00 mg/ml and no more than 3.00 mg/ml;
cefsulodin is at least 1.4 mg/ml and no more than 2.0 mg/ml;
ferric chloride is from 10 mg/L to 10 g/L;
the sulphite compound is at least 5 g/L and no more than 50 g/L.

24. The composition according to claim 21, wherein the composition is lyophilised.

25. A method for quantifying or detecting the presence or absence of Salmonella or E. coli in a test sample, the method comprising:
selectively culturing microbial cells present in the test sample; and
quantifying or detecting the presence or absence of the Salmonella or E. coli;
wherein the test sample comprises a food product, a clinical sample, a swab, a cosmetic, raw meat, raw poultry, a swab taken from raw meat, a swab taken from raw poultry, non-microbial material, or liquid in which a sample substance is suspended.

26-34. (canceled)

35. A kit for the selective culture of Salmonella cells or E. coli cells that may be present in a test sample, wherein the kit comprises:
a) an agent of Formula I as defined in claim 1; and
cefsulodin, novobiocin, ferric chloride, or a sulphite compound;
b) an agent of Formula I as defined in claim 1;
cefsulodin; and
novobiocin; or
c) an agent of Formula I as defined in claim 1;
cefsulodin;
novobiocin;
ferric chloride; and
a sulphite compound.

36-39. (canceled)

40. The method of claim 1, wherein where A is 2PY, 3PY, 4PY PZ QN or HD, R is selected from the group consisting of:

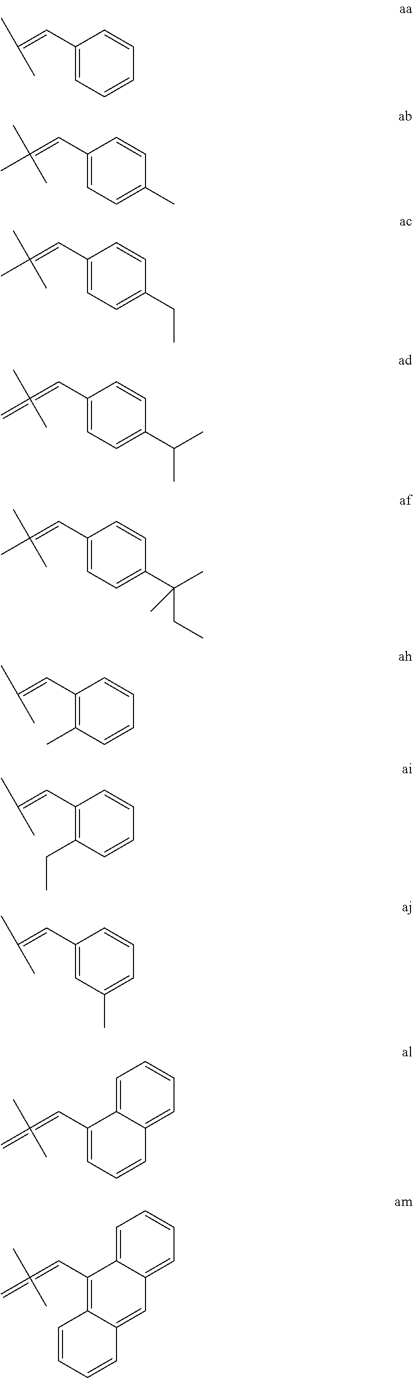

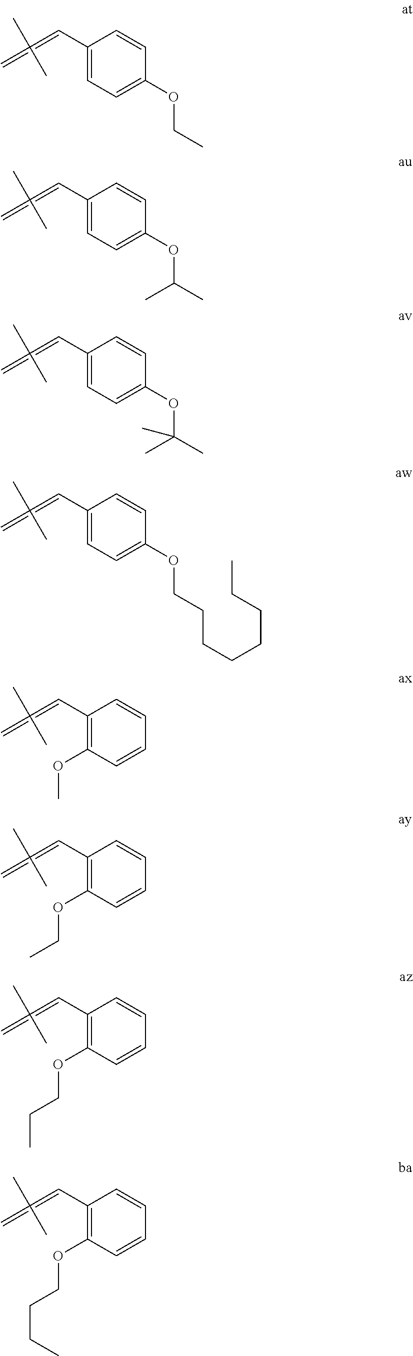

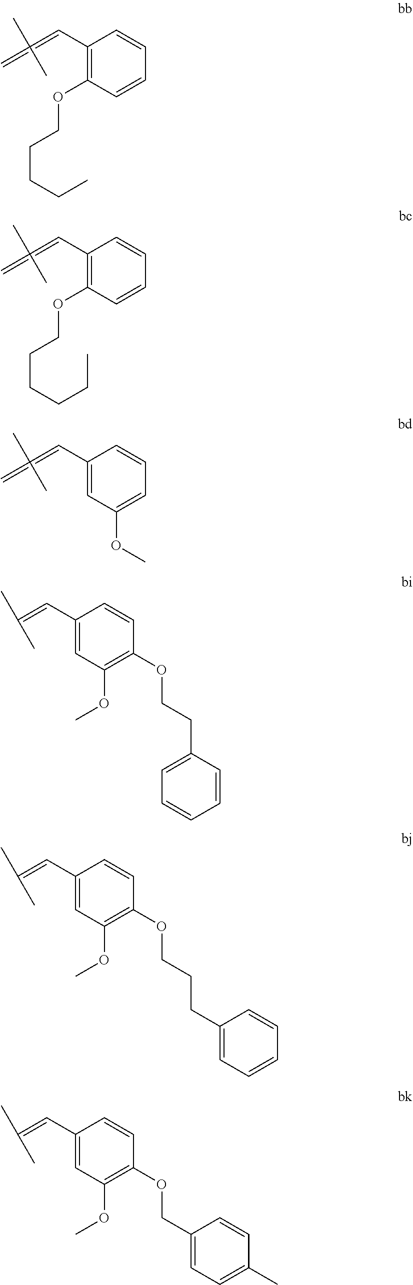

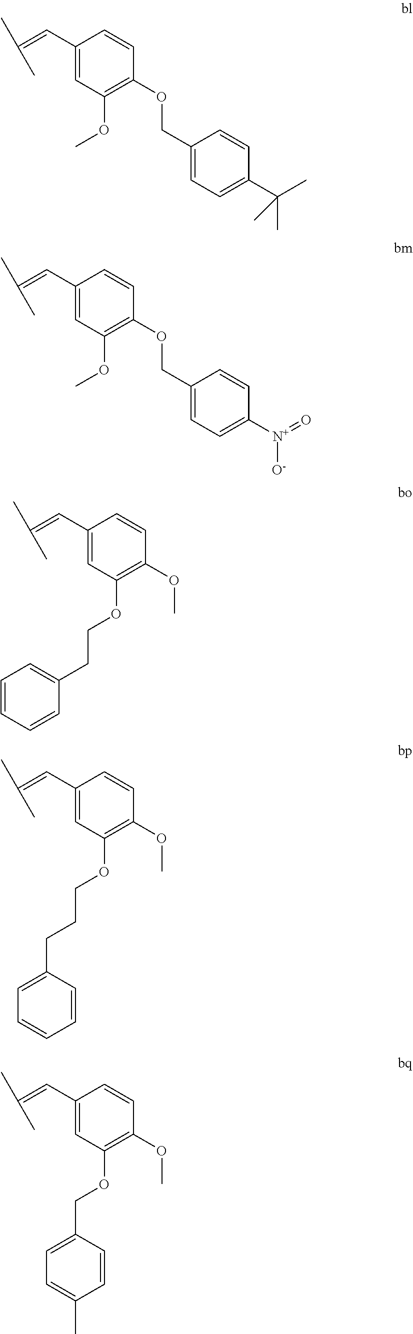

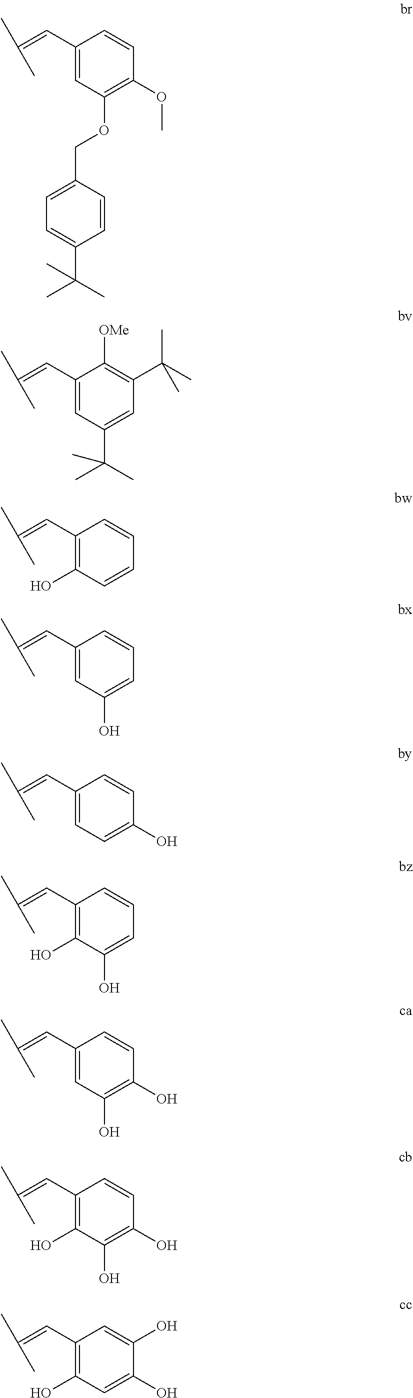

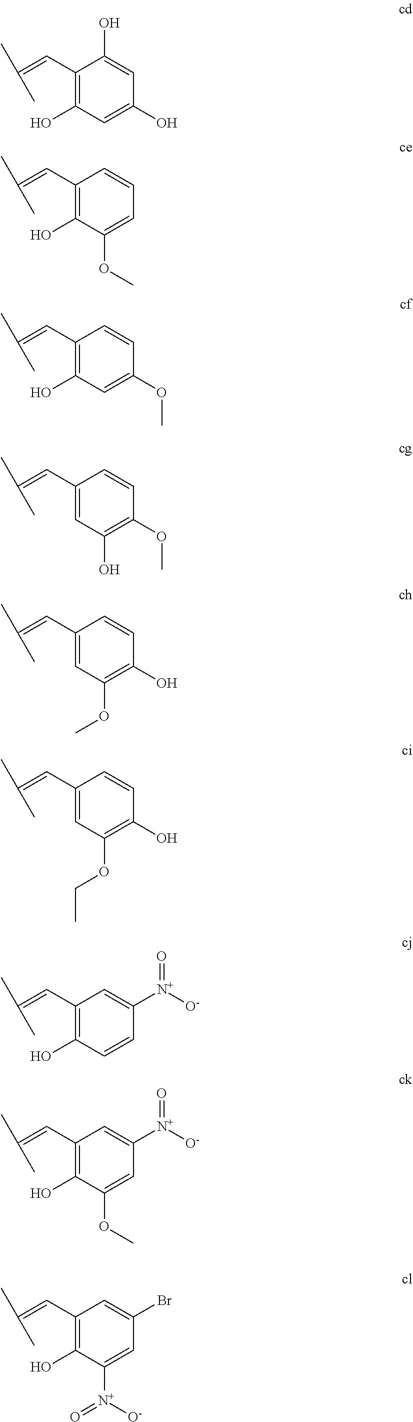

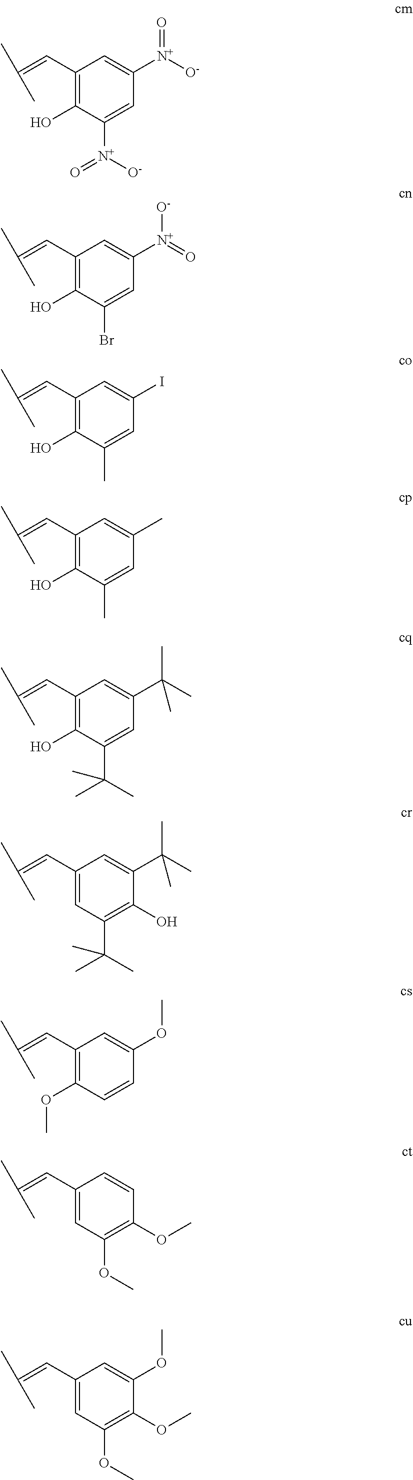

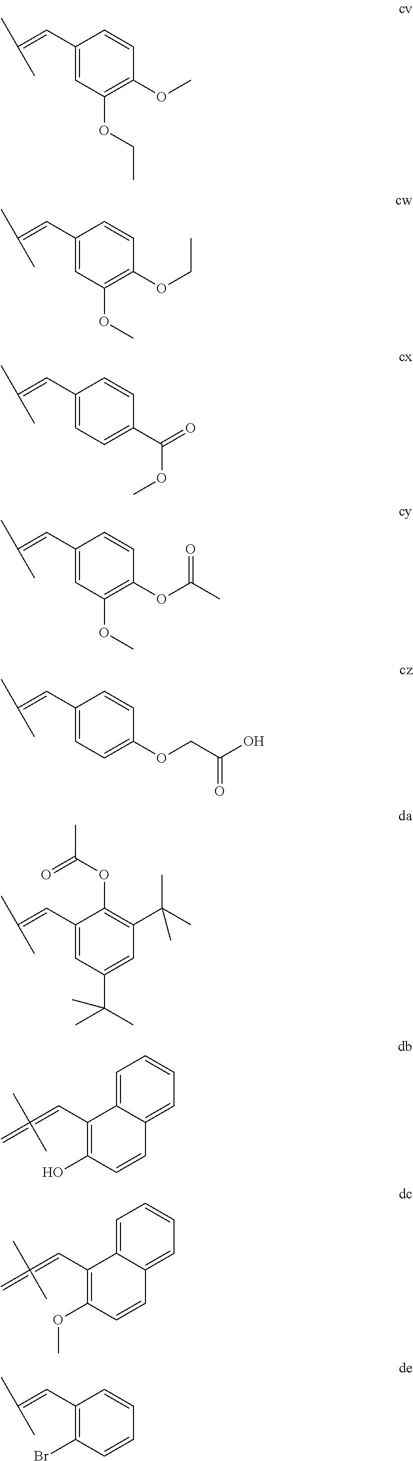

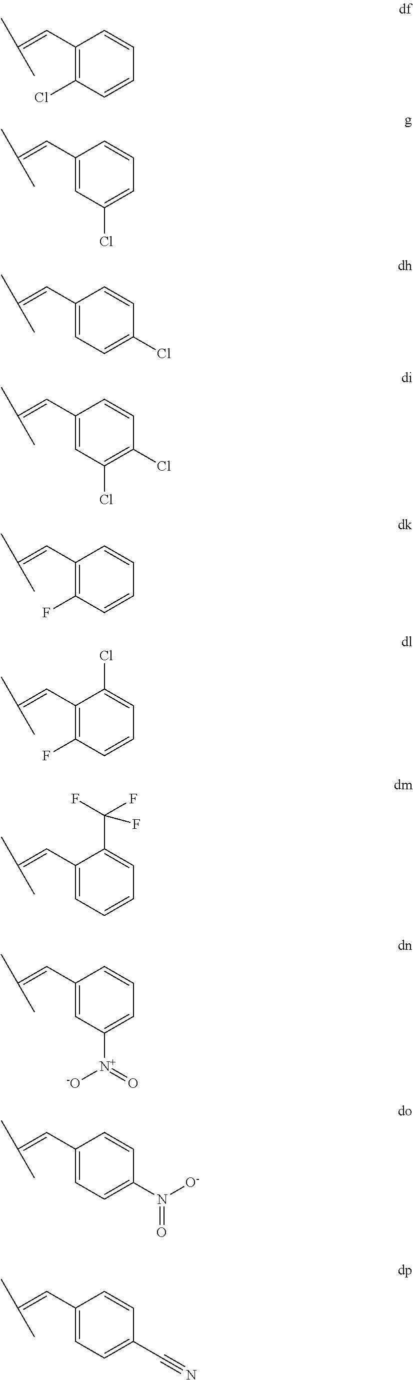

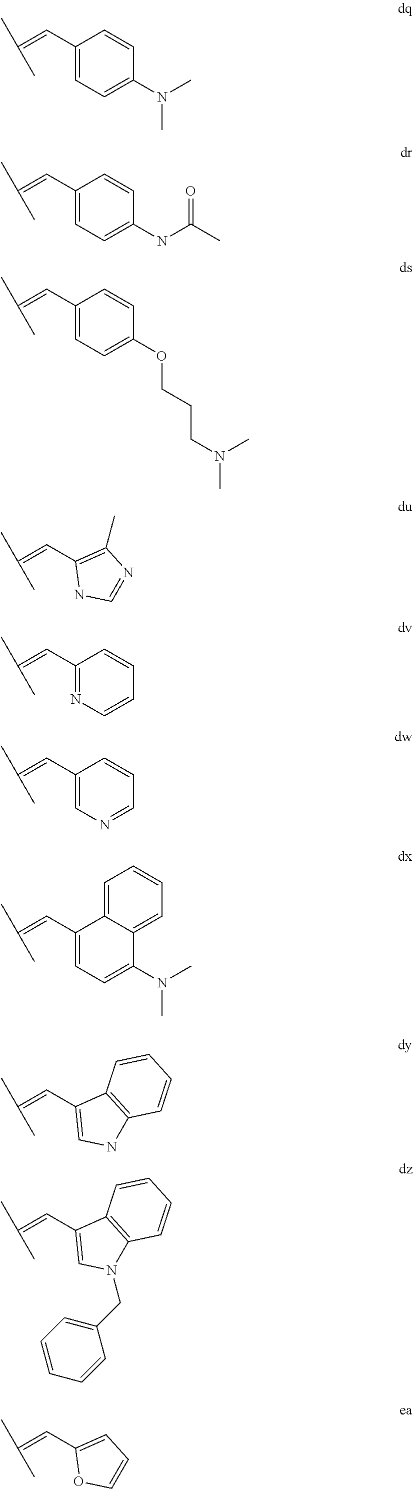

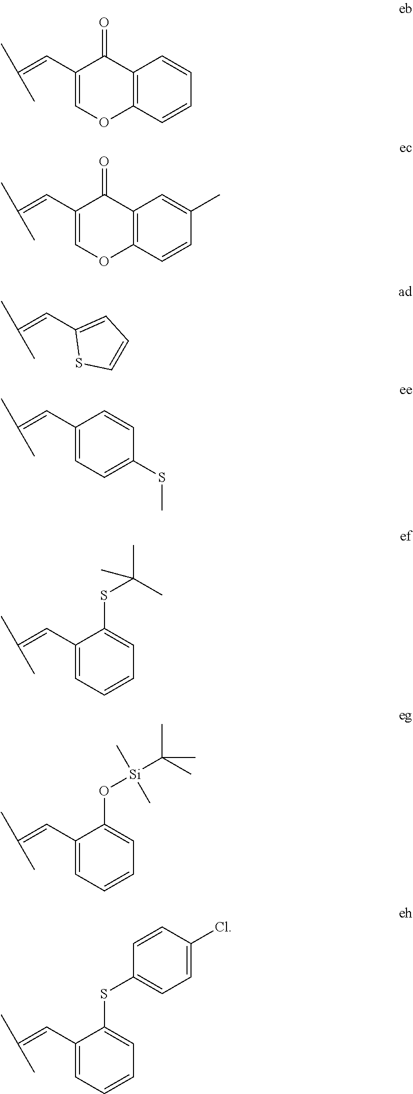

41. The method of claim 40, wherein the test sample comprises a food product, a clinical sample, a therapeutic product, a swab, a cosmetic, raw meat, raw poultry, a swab taken from raw meat, a swab taken from raw poultry, non-microbial material, or a suspension of any preceding sample in a liquid.

42. The culture medium of claim 18, wherein the concentration of ferric chloride is at least 0.25 mg/L and no more than 4 mg/L.

43. The culture medium of claim 18, wherein the concentration of the sulphite compound is at least 0.5 g/L and no more than 4.0 g/L.

44. The kit of claim 35, wherein the agent of Formula I is 4PYcq.
